SEQUENCE LISTING (238 sequences)

SEQ ID NO: 1 - protein, 31 aa, Homo sapiens
EAEDLQVGQVELGGGPGAGSLQPLALEGSLQ

SEQ ID NO: 2 - protein, 12 aa, Homo sapiens
EAEDLQVGQVEL

SEQ ID NO: 3 - protein, 13 aa, Homo sapiens
GGGPGAGSLQPLA

SEQ ID NO: 4 - protein, 6 aa, Homo sapiens
LEGSLQ

SEQ ID NO: 5 - protein, 4 aa, Homo sapiens
EAED

SEQ ID NO: 6 - protein, 5 aa, Homo sapiens
EGSLQ

SEQ ID NO: 7 - protein, 5 aa, Rattus norvegicus
EVARQ

SEQ ID NO: 8 - protein, 7 aa, Homo sapiens
GGGPGAG

SEQ ID NO: 9 - protein, 9 aa, Homo sapiens
ELGGGPGAG

SEQ ID NO: 10 - protein, 28 aa, Homo sapiens
DLQVGQVELGGGPGAGSLQPLALEGSLQ

SEQ ID NO: 11 - protein, 27 aa, Homo sapiens
LQVGQVELGGGPGAGSLQPLALEGSLQ

SEQ ID NO: 12 - protein, 25 aa, Homo sapiens
VGQVELGGGPGAGSLQPLALEGSLQ

SEQ ID NO: 13 - protein, 24 aa, Homo sapiens
GQVELGGGPGAGSLQPLALEGSLQ

SEQ ID NO: 14 - protein, 20 aa, Homo sapiens
LGGGPGAGSLQPLALEGSLQ

SEQ ID NO: 15 - protein, 20 aa, Homo sapiens
VGQVELGGGPGAGSLQPLAL

SEQ ID NO: 16 - protein, 19 aa, Homo sapiens
EVGQVELGGGPGAGSLQPL

SEQ ID NO: 17 - protein, 26 aa, Homo sapiens
EAEDLQVGQVELGGGPGAGSLQPLAL

SEQ ID NO: 18 - protein, 24 aa, Homo sapiens
EAEDLQVGQVELGGGPGAGSLQPL

SEQ ID NO: 19 - protein, 22 aa, Homo sapiens
LQVGQVELGGGPGAGSLQPLAL

SEQ ID NO: 20 - protein, 21 aa, Homo sapiens
DLQVGQVELGGGPGAGSLQPL

SEQ ID NO: 21 - protein, 20 aa, Homo sapiens
LQVGQVELGGGPGAGSLQPL

SEQ ID NO: 22 - protein, 13 aa, Homo sapiens
LGGGPGAGSLQPL

SEQ ID NO: 23 - protein, 15 aa, Homo sapiens
VGQVELGGGPGAGSL

SEQ ID NO: 24 - protein, 10 aa, Homo sapiens
GGGPGAGSLQ

SEQ ID NO: 25 - protein, 19 aa, Homo sapiens
FRAAPLQGMLPGLLAPLRT

SEQ ID NO: 26 - protein, 15 aa, Homo sapiens
AAPLQGMLPGLLAPL

SEQ ID NO: 27 - protein, 12 aa, Homo sapiens
LQGMLPGLLAPL

SEQ ID NO: 28 - protein, 10 aa, Homo sapiens
LQGMLPGLLA

SEQ ID NO: 29 - protein, 7 aa, Homo sapiens
LQGMLPG

SEQ ID NO: 30 - protein, 8 aa, Homo sapiens
GMLPGLLA

SEQ ID NO: 31 - protein, 4 aa, Homo sapiens
QDEA

SEQ ID NO: 32 - protein, 8 aa, Homo sapiens
LGGGPGAG

SEQ ID NO: 33 - protein, 8 aa, Bos taurus
LAGGPGAG

SEQ ID NO: 34 - protein, 6 aa, Homo sapiens
LGGGPG

SEQ ID NO: 35 - protein, 6 aa, Bos taurus
LAGGPG

SEQ ID NO: 36 - protein, 5 aa, Homo sapiens
LGGGP

SEQ ID NO: 37 - protein, 5 aa, Bos taurus
LAGGP

SEQ ID NO: 38 - protein, 4 aa, Homo sapiens
GGGP

SEQ ID NO: 39 - protein, 4 aa, synthetic construct
  REGION 1..4: note = Retro sequence peptide
GAGP

SEQ ID NO: 40 - protein, 5 aa, Homo sapiens
GGGPG

SEQ ID NO: 41 - protein, 6 aa, Homo sapiens
VGVAPG

SEQ ID NO: 42 - protein, 10 aa, Homo sapiens
ELGGGPGAGS

SEQ ID NO: 43 - protein, 6 aa, synthetic construct
  REGION 1..6: note = Immunomodulatory peptide
QGQLPG

SEQ ID NO: 44 - protein, 6 aa, Homo sapiens
  REGION 1..6: note = selected from human Galectin-3
PGAYPG

SEQ ID NO: 45 - protein, 6 aa, Homo sapiens
  REGION 1..6: note = selected from loop 2 of human beta-hCG
QGVLPA

SEQ ID NO: 46 - protein, 5 aa, Bos taurus
GAGPG

SEQ ID NO: 47 - protein, 7 aa, Homo sapiens
LGGGPGA

SEQ ID NO: 48 - protein, 6 aa, Homo sapiens
GGPGAG

SEQ ID NO: 49 - protein, 6 aa, Homo sapiens
GGGPGA

SEQ ID NO: 50 - protein, 5 aa, Homo sapiens
GGGPE

SEQ ID NO: 51 - protein, 5 aa, Homo sapiens
GAIPG

SEQ ID NO: 52 - protein, 5 aa, Homo sapiens
GGVPG

SEQ ID NO: 53 - protein, 5 aa, Homo sapiens
GVAPG

SEQ ID NO: 54 - protein, 14 aa, Homo sapiens
YTTGKLPYGYGPGG

SEQ ID NO: 55 - protein, 12 aa, Homo sapiens
YGARPGVGVGIP

SEQ ID NO: 56 - protein, 9 aa, Homo sapiens
PGFGAVPGA

SEQ ID NO: 57 - protein, 5 aa, Homo sapiens
GVYPG

SEQ ID NO: 58 - protein, 5 aa, Homo sapiens
GFGPG

SEQ ID NO: 59 - protein, 5 aa, Homo sapiens
GVLPG

SEQ ID NO: 60 - protein, 6 aa, Homo sapiens
PGAIPG

SEQ ID NO: 61 - protein, 6 aa, Homo sapiens
PGAVGP

SEQ ID NO: 62 - protein, 6 aa, Homo sapiens
VGAMPG

SEQ ID NO: 63 - protein, 6 aa, Homo sapiens
VGSLPG

SEQ ID NO: 64 - protein, 6 aa, Homo sapiens
VGMAPG

SEQ ID NO: 65 - protein, 5 aa, Homo sapiens
VPGVG

SEQ ID NO: 66 - protein, 5 aa, Homo sapiens
IPGVG

SEQ ID NO: 67 - protein, 5 aa, Homo sapiens
VGVPG

SEQ ID NO: 68 - protein, 6 aa, Homo sapiens
AGAIPG

SEQ ID NO: 69 - protein, 4 aa, Homo sapiens
VPGV

SEQ ID NO: 70 - protein, 6 aa, Homo sapiens
LGITPG

SEQ ID NO: 71 - protein, 4 aa, Homo sapiens
GDNP

SEQ ID NO: 72 - protein, 4 aa, Homo sapiens
GAIP

SEQ ID NO: 73 - protein, 4 aa, Homo sapiens
GKVP

SEQ ID NO: 74 - protein, 4 aa, Homo sapiens
GVQY

SEQ ID NO: 75 - protein, 4 aa, Homo sapiens
GVLP

SEQ ID NO: 76 - protein, 4 aa, Homo sapiens
GVGP

SEQ ID NO: 77 - protein, 4 aa, Homo sapiens
GFGP

SEQ ID NO: 78 - protein, 4 aa, Homo sapiens
GGIP

SEQ ID NO: 79 - protein, 4 aa, Homo sapiens
GVAP

SEQ ID NO: 80 - protein, 4 aa, Homo sapiens
GIGP

SEQ ID NO: 81 - protein, 5 aa, Homo sapiens
GGIPP

SEQ ID NO: 82 - protein, 4 aa, Homo sapiens
GQFP

SEQ ID NO: 83 - protein, 4 aa, Homo sapiens
GLSP

SEQ ID NO: 84 - protein, 4 aa, Homo sapiens
GPQP

SEQ ID NO: 85 - protein, 5 aa, Homo sapiens
GGPQP

SEQ ID NO: 86 - protein, 5 aa, Homo sapiens
GPQPG

SEQ ID NO: 87 - protein, 6 aa, Homo sapiens
GGPQPG

SEQ ID NO: 88 - protein, 4 aa, Homo sapiens
GIPP

SEQ ID NO: 89 - protein, 5 aa, Homo sapiens
GIPPA

SEQ ID NO: 90 - protein, 6 aa, Homo sapiens
GGIPPA

SEQ ID NO: 91 - protein, 58 aa, Homo sapiens
GGYPGASYPGAYPGQAPPGAYPGQAPPGAYPGAPGAYPGAPAPGVYPGPPSGPGAYPS

SEQ ID NO: 92 - protein, 9 aa, Homo sapiens
GGYPGASYP

SEQ ID NO: 93 - protein, 9 aa, Homo sapiens
GAYPGQAPP

SEQ ID NO: 94 - protein, 7 aa, Homo sapiens
GAYPGQA

SEQ ID NO: 95 - protein, 7 aa, Homo sapiens
GAYPGAP

SEQ ID NO: 96 - protein, 5 aa, Homo sapiens
GAYPG

SEQ ID NO: 97 - protein, 9 aa, Homo sapiens
APAPGVYPG

SEQ ID NO: 98 - protein, 5 aa, Homo sapiens
GAYPS

SEQ ID NO: 99 - protein, 40 aa, Homo sapiens
QTLPGSCGQVVGSPSAQDEASPLSEWRASYNSAGSNITDA

SEQ ID NO: 100 - protein, 32 aa, Homo sapiens
LPGSCGQVVGSPSAQDEASPLSEWRASYNSAG

SEQ ID NO: 101 - protein, 21 aa, Homo sapiens
VVGSPSAQDEASPLSEWRASY

SEQ ID NO: 102 - protein, 15 aa, Homo sapiens
VVGSPSAQDEASPLS

SEQ ID NO: 103 - protein, 10 aa, Homo sapiens
PSAQDEASPL

SEQ ID NO: 104 - protein, 10 aa, Homo sapiens
SPSAQDEASP

SEQ ID NO: 105 - protein, 6 aa, Homo sapiens
AQDEAS

SEQ ID NO: 106 - protein, 4 aa, Homo sapiens
PSAQ

SEQ ID NO: 107 - protein, 4 aa, Homo sapiens
SAQD

SEQ ID NO: 108 - protein, 4 aa, Homo sapiens
DEAS

SEQ ID NO: 109 - protein, 4 aa, Homo sapiens
SPSA

SEQ ID NO: 110 - protein, 15 aa, synthetic construct
  REGION 1..15: note = antagonist peptide
VVGGTEAQRNSWPLQ

SEQ ID NO: 111 - protein, 15 aa, synthetic construct
  REGION 1..15: note = antagonist peptide
VVGGTEAQRNSWPSQ

SEQ ID NO: 112 - protein, 9 aa, synthetic construct
  REGION 1..9: note = antagonist peptide
TEAQRNSWP

SEQ ID NO: 113 - protein, 4 aa, synthetic construct
  REGION 1..4: note = antagonist peptide
AQRN

SEQ ID NO: 114 - protein, 15 aa, synthetic construct
  REGION 1..15: note = antagonist peptide
IVGGRRARPHAWPFM

SEQ ID NO: 115 - protein, 15 aa, synthetic construct
  REGION 1..15: note = antagonist peptide
VVGGEDAKPGQFPWQ

SEQ ID NO: 116 - protein, 15 aa, synthetic construct
  REGION 1..15: note = antagonist peptide
VVGGRVAQPNSWPWQ

SEQ ID NO: 117 - protein, 8 aa, synthetic construct
  REGION 1..8: note = antagonist peptide
RVAQPNSW

SEQ ID NO: 118 - protein, 15 aa, synthetic construct
  REGION 1..15: note = antagonist peptide
VVGGAEARRNSWPSQ

SEQ ID NO: 119 - protein, 8 aa, synthetic construct
  REGION 1..8: note = antagonist peptide
AEARRNSW

SEQ ID NO: 120 - protein, 15 aa, synthetic construct
  REGION 1..15: note = antagonist peptide
VVGGQEATPNTWPWQ

SEQ ID NO: 121 - protein, 8 aa, synthetic construct
  REGION 1..8: note = antagonist peptide
QEATPNTW

SEQ ID NO: 122 - protein, 15 aa, synthetic construct
  REGION 1..15: note = antagonist peptide
VVGGEEARPNSWPWQ

SEQ ID NO: 123 - protein, 8 aa, synthetic construct
  REGION 1..8: note = antagonist peptide
EEARPNSW

SEQ ID NO: 124 - protein, 15 aa, synthetic construct
  REGION 1..15: note = antagonist
VVGGTEAGRNSWPSQ

SEQ ID NO: 125 - protein, 9 aa, synthetic construct
  REGION 1..9: note = antagonist
TEAGRNSWP

SEQ ID NO: 126 - protein, 15 aa, synthetic construct
  REGION 1..15: note = antagonist peptide
EDYRPSQQDECSPRE

SEQ ID NO: 127 - protein, 9 aa, synthetic construct
  REGION 1..9: note = antagonist peptide
PSQQDECSP

SEQ ID NO: 128 - protein, 5 aa, synthetic construct
  REGION 1..5: note = antagonist peptide
QQDEC

SEQ ID NO: 129 - protein, 8 aa, Homo sapiens
LALEGSLQ

SEQ ID NO: 130 - protein, 4 aa, Homo sapiens
AGGP

SEQ ID NO: 131 - protein, 14 aa, Homo sapiens
VVGSPSAQDEASPL

SEQ ID NO: 132 - skipped

SEQ ID NO: 133 - protein, 7 aa, Homo sapiens
GGGPGAG

SEQ ID NO: 134 - protein, 6 aa, Rattus norvegicus
LGGGPE

SEQ ID NO: 135 - protein, 6 aa, Sus scrofa
LGGGLG

SEQ ID NO: 136 - protein, 31 aa, synthetic construct
  REGION 1..31: note = retro C-Peptide
QLSGELALPQLSGAGPGGGLEVQGVQLDEAE

SEQ ID NO: 137 - protein, 6 aa, Homo sapiens
LGTIPG

SEQ ID NO: 138 - protein, 6 aa, synthetic construct
  REGION 1..6: note = scrambled peptide
GVAPGV

SEQ ID NO: 139 - protein, 6 aa, synthetic construct
  REGION 1..6: note = scrambled peptide
VAPGVG

SEQ ID NO: 140 - protein, 6 aa, synthetic construct
  REGION 1..6: note = scrambled peptide
APGVGV

SEQ ID NO: 141 - protein, 6 aa, synthetic construct
  REGION 1..6: note = scrambled peptide
PGVGVA

SEQ ID NO: 142 - protein, 6 aa, synthetic construct
  REGION 1..6: note = scrambled peptide
GVGVAP

SEQ ID NO: 143 - protein, 10 aa, Homo sapiens
VPGVGISPEA

SEQ ID NO: 144 - protein, 10 aa, Homo sapiens
GVAPGIGPGG

SEQ ID NO: 145 - protein, 14 aa, Homo sapiens
VELGGGPGAGSLQP

SEQ ID NO: 146 - protein, 12 aa, Homo sapiens
LGGGPGAGSLQP

SEQ ID NO: 147 - protein, 9 aa, Homo sapiens
LGGGPGAGS

SEQ ID NO: 148 - protein, 55 aa, Homo sapiens
AGAGGYPGASYPGAYPGQAPPGAYPGQAPPGAYPGAPGAYPGAPAPGVYPGPPSG

SEQ ID NO: 149 - protein, 6 aa, Homo sapiens
EGFEPG

SEQ ID NO: 150 - protein, 31 aa, Homo sapiens
EAEDLQVGQVEMGGGPGAGSLQPLALEGSLQ

SEQ ID NO: 151 - protein, 31 aa, Homo sapiens
EAEDLQVGQVELGGGPGAGSLQPLALERSLQ

SEQ ID NO: 152 - protein, 31 aa, Hylobates sp.
EAEDPQVGQVELGGGPGAGSLQPLALEGSLQ

SEQ ID NO: 153 - protein, 31 aa, Mus musculus
EVEDPQVAQLELGGGPGAGDLQTLALEVAQQ

SEQ ID NO: 154 - protein, 31 aa, Rattus norvegicus
EVEDPQVAQLELGGGPGAGDLQTLALEVARQ

SEQ ID NO: 155 - protein, 31 aa, Rattus norvegicus
EVEDPQVPQLELGGGPEAGDLQTLALEVARQ

SEQ ID NO: 156 - protein, 31 aa, Equus caballus
EAEDPQVGQEELGGGPGLGGLQPLALAGPQQ

SEQ ID NO: 157 - protein, 31 aa, Equus caballus
EAEDPQVGEVELGGGPGLGGLQPLALAGPQQ

SEQ ID NO: 158 - protein, 31 aa, Equus caballus
EAEDPQVGQVELGGGPGLGGLQPLALAGPQQ

SEQ ID NO: 159 - protein, 31 aa, Chinchilla lanigera
ELEDPQVGQADPGVVPEAGRLQPLALEMTLQ

SEQ ID NO: 160 - protein, 31 aa, Cavia porcellus
ELEDPQVEQTELGMGLGAGGLQPLALEMALQ

SEQ ID NO: 161 - protein, 31 aa, Oryctolagus cuniculus
EVEELQVGQAELGGGPGAGGLQPSALELALQ

SEQ ID NO: 162 - protein, 31 aa, Canis lupus
EVEDLQVRDVELAGAPGEGGLQPLALEGALQ

SEQ ID NO: 163 - protein, 31 aa, Felis catus
EAEDLQGKDAELGEAPGAGGLQPSALEAPLQ

SEQ ID NO: 164 - protein, 26 aa, Bos taurus
EVEGPQVGALELAGGPGAGGLEGPPQ

SEQ ID NO: 165 - protein, 26 aa, Bos taurus
  REGION 1..26: note = Fleckvieh variant
EVEGPQVGALELAGGLGAGGLEGPPQ

SEQ ID NO: 166 - protein, 29 aa, Sus scrofa
EAENPQAGAVELGGGLGGLQALALEGPPQ

SEQ ID NO: 167 - protein, 29 aa, Mus musculus
EVEDPQVEQLELGGSPGDLQTLALEVARQ

SEQ ID NO: 168 - protein, 20 aa, Homo sapiens
PVLPVPPGFPPGPQIPVPRP

SEQ ID NO: 169 - protein, 11 aa, Homo sapiens
TCEEGFEPGPM

SEQ ID NO: 170 - protein, 41 aa, Homo sapiens
GLVPGVGVAPGVGVAPGVGVAPGVGLAPGVGVAPGVGVAPG

SEQ ID NO: 171 - protein, 20 aa, Homo sapiens
LPMGGIPGSAGSRPGGTGGN

SEQ ID NO: 172 - protein, 11 aa, Homo sapiens
NCNEGFEPGPM

SEQ ID NO: 173 - protein, 31 aa, Psammomys obesus
GVDDPQMPQLELGGSPGAGDLRALALEVARQ

SEQ ID NO: 174 - protein, 31 aa, Heterocephalus glaber
ELENLQVGQAEPGMGLEAGGLQPLAQELALQ

SEQ ID NO: 175 - protein, 18 aa, Homo sapiens
VGQVELGGGPGAGSLQPL

SEQ ID NO: 176 - protein, 14 aa, Homo sapiens
ELGGGPGAGSLQPL

SEQ ID NO: 177 - protein, 33 aa, Homo sapiens
CGNLSTCMLGTYTQDFNKFHTFPQTAIGVGAPG

SEQ ID NO: 178 - protein, 26 aa, Homo sapiens
MLGTYTQDFNKFHTFPQTAIGVGAPG

SEQ ID NO: 179 - protein, 18 aa, Homo sapiens
FNKFHTFPQTAIGVGAPG

SEQ ID NO: 180 - protein, 12 aa, Homo sapiens
FPQTAIGVGAPG

SEQ ID NO: 181 - protein, 8 aa, Homo sapiens
AIGVGAPG

SEQ ID NO: 182 - protein, 21 aa, Homo sapiens
SHPLGSPGSASDLETSGLQEQ

SEQ ID NO: 183 - protein, 19 aa, Homo sapiens
PLGSPGSASDLETSGLQEQ

SEQ ID NO: 184 - protein, 14 aa, Homo sapiens
PLGSPGSASDLETS

SEQ ID NO: 185 - protein, 9 aa, Homo sapiens
PLGSPGSAS

SEQ ID NO: 186 - protein, 6 aa, Homo sapiens
PLGSPG

SEQ ID NO: 187 - protein, 31 aa, Homo sapiens
EDVSAGEDCGPLPEGGPEPRSDGAKPGPREG

SEQ ID NO: 188 - protein, 26 aa, Homo sapiens
GEDCGPLPEGGPEPRSDGAKPGPREG

SEQ ID NO: 189 - protein, 21 aa, Homo sapiens
PLPEGGPEPRSDGAKPGPREG

SEQ ID NO: 190 - protein, 17 aa, Homo sapiens
PLPEGGPEPRSDGAKPG

SEQ ID NO: 191 - protein, 7 aa, Homo sapiens
SDGAKPG

SEQ ID NO: 192 - protein, 24 aa, Homo sapiens
RRNASSAGRLQGLAGGAPGQKECR

SEQ ID NO: 193 - protein, 16 aa, Homo sapiens
RLQGLAGGAPGQKECR

SEQ ID NO: 194 - protein, 20 aa, Homo sapiens
RRNASSAGRLQGLAGGAPGQ

SEQ ID NO: 195 - protein, 11 aa, Homo sapiens
LQGLAGGAPGQ

SEQ ID NO: 196 - protein, 6 aa, Homo sapiens
AGGAPG

SEQ ID NO: 197 - protein, 6 aa, Homo sapiens
YGYGPG

SEQ ID NO: 198 - protein, 6 aa, Homo sapiens
YGARPG

SEQ ID NO: 199 - protein, 6 aa, Homo sapiens
FGAVPG

SEQ ID NO: 200 - protein, 6 aa, Homo sapiens
GVGAPG

SEQ ID NO: 201 - protein, 6 aa, Homo sapiens
DGAKPG

SEQ ID NO: 202 - protein, 6 aa, Homo sapiens
QGMLPG

SEQ ID NO: 203 - protein, 10 aa, Homo sapiens
FGLVPGVGVA

SEQ ID NO: 204 - protein, 10 aa, Homo sapiens
GVAPGIGPGG

SEQ ID NO: 205 - protein, 28 aa, Homo sapiens
PPGAYPGQAPPGAYPGAPGAYPGAPAPG

SEQ ID NO: 206 - protein, 10 aa, Homo sapiens
TCEEGFEPGP

SEQ ID NO: 207 - protein, 10 aa, Homo sapiens
NPLGTIPGGN

SEQ ID NO: 208 - protein, 35 aa, Homo sapiens
RREAEDLQVGQVELGGGPGAGSLQPLALEGSLQKR

SEQ ID NO: 209 - protein, 18 aa, Homo sapiens
RVLQGVLPALPQVVCNYR

SEQ ID NO: 210 - protein, 38 aa, Homo sapiens
KRCGNLSTCMLGTYTQDFNKFHTFPQTAIGVGAPGKKR

SEQ ID NO: 211 - protein, 23 aa, Homo sapiens
RSHPLGSPGSASDLETSGLQEQR

SEQ ID NO: 212 - protein, 35 aa, Homo sapiens
KREDVSAGEDCGPLPEGGPEPRSDGAKPGPREGKR

SEQ ID NO: 213 - protein, 17 aa, Homo sapiens
RAAPLQGMLPGLLAPLR

SEQ ID NO: 214 - protein, 6 aa, Homo sapiens
FGLVPG

SEQ ID NO: 215 - protein, 6 aa, Homo sapiens
PGIGPG

SEQ ID NO: 216 - protein, 42 aa, Homo sapiens
VGVAPGVGVAPGVGVAPGVGLAPGVGVAPGVGVAPGVGVAPG

SEQ ID NO: 217 - protein, 9 aa, Homo sapiens
VGVLPGVPT

SEQ ID NO: 218 - protein, 7 aa, Homo sapiens
GLGAFPA

SEQ ID NO: 219 - protein, 8 aa, synthetic construct
  SITE 2: note = D-alanine
  SITE 4: note = D-proline
  SITE 8: note = D-leucine
GAGPGGGL

SEQ ID NO: 220 - protein, 7 aa, synthetic construct
  SITE 2: note = D-alanine
  SITE 4: note = D-proline
GAGPGGG

SEQ ID NO: 221 - protein, 7 aa, synthetic construct
  SITE 1: note = D-alanine
  SITE 3: note = D-proline
  SITE 7: note = D-leucine
AGPGGGL

SEQ ID NO: 222 - protein, 6 aa, synthetic construct
  SITE 2: note = D-alanine
  SITE 4: note = D-proline
GAGPGG

SEQ ID NO: 223 - protein, 6 aa, synthetic construct
  SITE 2: note = D-proline
  SITE 6: note = D-leucine
GPGGGL

SEQ ID NO: 224 - protein, 6 aa, synthetic construct
  SITE 1: note = D-alanine
  SITE 3: note = D-proline
AGPGGG

SEQ ID NO: 225 - protein, 5 aa, synthetic construct
  SITE 2: note = D-proline
GPGGG

SEQ ID NO: 226 - protein, 5 aa, synthetic construct
  SITE 2: note = D-proline
  SITE 4: note = D-alanine
GPGAG

SEQ ID NO: 227 - protein, 4 aa, synthetic construct
  SITE 1: note = D-proline
PGGG

SEQ ID NO: 228 - protein, 4 aa, synthetic construct
  SITE 1: note = D-proline
  SITE 3: note = D-alanine
PGAG

SEQ ID NO: 229 - protein, 4 aa, synthetic construct
  SITE 1: note = D-alanine
  SITE 2: note = D-glutamic acid
  SITE 3: note = D-aspartic acid
  SITE 4: note = D-glutamine
AEDQ

SEQ ID NO: 230 - protein, 14 aa, synthetic construct
  SITE 1: note = D-leucine
  SITE 2: note = D-proline
  SITE 3: note = D-serine
  SITE 4: note = D-alanine
  SITE 5: note = D-glutamic acid
  SITE 6: note = D-aspartic acid
  SITE 7: note = D-glutamine
  SITE 8: note = D-alanine
  SITE 9: note = D-serine
  SITE 10: note = D-proline
  SITE 11: note = D-serine
  SITE 13: note = D-valine
  SITE 14: note = D-valine
LPSAEDQASPSGVV

SEQ ID NO: 231 - protein, 10 aa, synthetic construct
  SITE 1: note = D-proline
  SITE 2: note = D-serine
  SITE 3: note = D-alanine
  SITE 4: note = D-glutamic acid
  SITE 5: note = D-aspartic acid
  SITE 6: note = D-glutamine
  SITE 7: note = D-alanine
  SITE 8: note = D-serine
  SITE 9: note = D-proline
  SITE 10: note = D-serine
PSAEDQASPS

SEQ ID NO: 232 - protein, 20 aa, synthetic construct
  SITE 1: note = D-tryptophan
  SITE 2: note = D-glutamic acid
  SITE 3: note = D-serine
  SITE 4: note = D-leucine
  SITE 5: note = D-proline
  SITE 6: note = D-serine
  SITE 7: note = D-alanine
  SITE 8: note = D-glutamic acid
  SITE 9: note = D-aspartic acid
  SITE 10: note = D-glutamine
  SITE 11: note = D-alanine
  SITE 12: note = D-serine
  SITE 13: note = D-proline
  SITE 14: note = D-serine
  SITE 16: note = D-valine
  SITE 17: note = D-valine
  SITE 18: note = D-glutamine
  SITE 20: note = D-cysteine
WESLPSAEDQASPSGVVQGC

SEQ ID NO: 233 - protein, 8 aa, synthetic construct
  SITE 2: note = D-alanine
  SITE 4: note = D-proline
  SITE 7: note = D-alanine
  SITE 8: note = D-leucine
GAGPGGAL

SEQ ID NO: 234 - protein, 7 aa, synthetic construct
  SITE 2: note = D-proline
  SITE 6: note = D-proline
  SITE 7: note = D-alanine
GPGGGPA

SEQ ID NO: 235 - protein, 6 aa, synthetic construct
  SITE 2: note = D-proline
  SITE 5: note = D-alanine
  SITE 6: note = D-leucine
GPGGAL

SEQ ID NO: 236 - protein, 4 aa, Homo sapiens
LQGV

SEQ ID NO: 237 - protein, 4 aa, Homo sapiens
AQGV

SEQ ID NO: 238 - protein, 26 aa, Homo sapiens
EAEDLQVGQVELGGGPGAGSLQPLAL

(19) United States
(12) Patent Application Publication
(10) Pub. No.: US 2024/0027431 A1
(43) Pub. Date: Jan. 25, 2024

(54) PEPTIDE MODULATORS OF THE INTERACTION BETWEEN HUMAN C-PEPTIDE AND HUMAN ELASTIN RECEPTOR FOR THERAPEUTIC USE

(71) Applicant: Biotempt B.V., The Hague (NL)

(72) Inventor: Gert Wensvoort, Koekange (NL)

(21) Appl. No.: 18/310,188

(22) Filed: May 1, 2023

Related U.S. Application Data
(62) Division of application No. 16/483,317, filed on Aug. 2, 2019, now abandoned.

(30) Foreign Application Priority Data
Feb. 6, 2017 (EP) 17154889.4

(51) Int. Cl. G01N 33/53; G01N 33/566; G01N 33/577

(52) U.S. Cl. CPC G01N 33/53; G01N 33/566; G01N 33/577; A61K 38/00

(57) ABSTRACT

The present disclosure shows that inflammation in metabolic syndrome is augmented by and hitherto overlooked lock-and-key activation of the elastin receptor, a protein involved in vascular (blood vessel) inflammation and elastin repair, with the C-peptide, a small protein that is produced in a 1:1 ratio alongside with widely known insulin. The elastin receptor is the lock that is activated by a key motif of amino acids (PG-domain) found in C-peptide and in breakdown products (PG-domain-fragments) thereof. Until now, no one has ever discovered this lock-and-key interaction between the two, now providing novel development of novel peptides for treatment of metabolic syndrome, exploiting the finding that not only the normal keys of the elastin receptor (elastin peptides), but also the C-peptide, a peptide we produce together with insulin every time glucose rises in our blood after a meal, interacts in a lock-and-key mode with the elastin receptor.

### CROSS-REFERENCE TO RELATED APPLICATIONS

This application is a divisional of U.S. patent application Ser. No. 16/483,317, filed Aug. 2, 2019, which is a national phase entry under 35 U.S.C. § 371 of International Patent Application PCT/EP2018/052822, filed Feb. 5, 2018, designating the United States of America and published as International Patent Publication WO 2018/141969 A1 on Aug. 9, 2018, which claims the benefit under Article 8 of the Patent Cooperation Treaty to European Patent Application Serial No. 17154889.4, filed Feb. 6, 2017, the disclosure of each of which is hereby incorporated herein in its entirety by this reference.

### TECHNICAL FIELD

The application relates generally to the field of human medicine, and belongs to the field of pharmacy, biotechnology, and drug development. The application relates to the etiology of metabolic syndrome and provides use of immune-modulatory peptides for treatment of inflammation, insulin resistance, atheromatous disease, arteriosclerosis, atherosclerosis, cardiovascular disease, micro- and macrovascular pathologies in type 1 and type 2 diabetes mellitus, in humans.

### BACKGROUND

Our all-too-human habit to overeat and the easy availability of everyday food have resulted in a worldwide obesity epidemic with dire consequences to our health. One-and-one-half (1.5) billion people are overweight (0.5 billion are obese), and a great many of those suffer from a chronic inflammatory disease often called metabolic syndrome: the major cause of unhealthy aging and death in high- and middle-income countries. These 1.5 billion people are at increased risk to develop cardiovascular disease (chronic inflammation of the blood vessels [atheromatous disease, arteriosclerosis, atherosclerosis], increased blood pressure [hypertension] and increased abnormal fat levels [dyslipidemia] in the blood), leading up to heart attack and stroke. At least 30% of these 1.5 billion are at further risk to develop diabetes type 2 (World Health Organization). Others develop too early manifestations of aging such as kidney failure or dementia. Heart failure, as non-fatal and fatal myocardial infarction and peripheral arterial disease (PAD) are the most common initial manifestations of cardiovascular disease in type 2 diabetes, others are transient ischemic attacks (TIA) or ischaemic stroke and stable angina. Living a sedentary life and smoking further increases risks of dying from these conditions. Currently, no satisfying medical understanding (other than excess diet) exists of the causal events leading to the initially mild but ultimately chronic inflammatory disease that underlies these staggering figures. Why this food-intake-induced inflammation occurs and affects so many people is largely unknown and human of much debate.

C-peptide is the linking peptide between the A- and B-chains in the proinsulin molecule. After cleavage in the endoplasmic reticulum of pancreatic islet beta-cells, insulin and a acid peptide are generated. The latter is processed to the 31-amino acid peptide, C-peptide, by enzymatic removal of two basic residues on either side of the molecule. C-peptide is co-secreted with insulin in equimolar amounts from the pancreatic islet beta-cells into the portal circulation. Besides its contribution to the folding of the two-chain insulin structure, further biologic activity of C-peptide was questioned for many years after its discovery.

Human C-peptide is a 31-amino acid peptide having the sequence SEQ ID NO:1 (EAEDLQVGQVELGGGPGAGSLQPLALEGSLQ, in the one-letter amino acid code).

C-peptide classically is ascribed a tripartite overall structure, with more conserved N- and C-terminal segments and a more variable mid-sequence, or internal, and hydrophobic midportion. Thus, in the case of human C-peptide the N-terminal segment is often regarded as residues 1-12 (SEQ ID NO:2 (EAEDLQVGQVEL)), the mid-portion as residues 13-(SEQ ID NO:3 (GGGPGAGSLQPLA)), and the C-terminal segment as residues 25-31 (SEQ ID NO:4 (LEGSLQ)). The tetrapeptide SEQ ID NO:5 (EAED) is thought to be required in the process of folding the two-chain insulin structure in the beta cells (Chen at al., J Biochem. 2002 June; 131(6):855-9). Recently, some studies suggested that the C-terminal pentapeptide (SEQ ID NO:6 EGSLQ) in human C-peptide and SEQ ID NO:7 (EVARQ) in rat C-peptide of C-peptide that shows a well-defined secondary structure may induce intracellular Ca2+ increases in human renal tubular cells (Shafqat et al., Cell Mol Life Sci. 2002 July; 59(7):1185-9).

C-peptide is produced in equal amounts to insulin and is the best additional measure of endogenous insulin secretion in patients with diabetes. Measurement of insulin secretion using C-peptide is considered helpful in clinical practice: differences in insulin secretion are fundamental to the different treatment requirements of Type 1 and Type 2 diabetes. Jones and Hattersley (Diabet Med. 2013 July; 30(7):803-17) review the use of C-peptide measurement in the clinical management of patients with diabetes, including the interpretation and choice of C-peptide test and its use to assist diabetes classification and choice of treatment, and recommendations for where C-peptide should be used, choice of test and interpretation of results. As the relationships between C-peptide levels and metabolic control and chronic complications are poorly known in type 2 diabetes, due to the slow decline of beta-cell function, Bo et al., (Acta Diabetol. 2000; 37(3):125-9) evaluated these associations in a cohort of type 2 diabetic patients. Biological effects of C-peptide are thought to be mediated by interaction with insulin or via specific or nonspecific membrane interaction. Some studies in the art support the theory of specific interactions with a yet to be identified GPCR. However, the D-enantiomer of C-peptide has the same biological activity as the L-enantiomer; (Ido et al., Science, 1997, 277(5325):563-6), thus finding reverse (retro) and all-D-amino acid (enantio) C-peptides equipotent to native C-peptide, IDO et al. conclude the activity of SEQ ID NO:8 (GGGPGAG) to be not mediated by a receptor, thereby teaching away from a receptor for C-peptide that has suggested to those in the art that other, receptor-independent, interactions are important for function of C-peptide. Formation of cation-selective channels in lipid bilayers has also led to suggestions of a more nonspecific interaction. Thus, a receptor for C-peptide has remained elusive.

Ido et al. (Science, 1997 Jul. 25; 277(5325):563-6; and FIG. 1 in this disclosure) show human C-peptide fragments with hydrophobic midportion SEQ ID NO:8 (GGGPGAG) to normalize glucose-induced vascular dysfunction in rat granular tissue, they however, have not provided testing of C-peptide fragments in combination with treatment of those rats with insulin, and teach away from receptor-mediated activity of fragments having the hydrophobic mid-portion. In US20020107175 a C-peptide fragment SEQ ID NO:9 (ELGGGPGAG) and some of its smaller fragments stimulate Na.sup.+K.sup.+ATPase activity of rat renal tubule cells. US20060234914 and 20070082842 list N-terminal- and/or C-terminal-C-peptide fragments that comprise at least one glutamine (in three letter code Glu; in one-letter code E) to provide biological activity not related to the activity of above discussed hydrophobic mid-portion SEQ ID NO:8 (GGGPGAG), which mid-portion sequence is not found in any of the fragments listed in US20060234914 nor 20070082842.

Type 1 diabetes is generally characterized by insulin and C-peptide deficiency, due to an autoimmune destruction of the pancreatic islet beta-cells. The patients are therefore dependent on exogenous insulin to sustain life. Several factors may be of importance for the pathogenesis of the disease, e.g., genetic background, environmental factors, and an aggressive autoimmune reaction following a temporary infection (Akerblom H K et al.: Annual Medicine 29(5): 383-385, (1997)). Currently insulin-requiring patients are provided with exogenous insulin that has been separated from the C-peptide, and thus do not receive exogenous C-peptide therapy. By contrast, most type 2 diabetic subjects initially still produce both insulin and C-peptide endogenously, but are generally characterized by insulin resistance in skeletal muscle, adipose tissue, and liver, among other tissues.

Many type 1 and end-phase type 2 diabetic patients (that do not longer produce insulin and C-peptide) and other insulin-requiring patients eventually develop and suffer from a constellation of long-term vascular complications of diabetes that in many cases are more severe and widespread than in early phase type 2 diabetes (wherein insulin and C-peptide are still produced, but the patient is resistant to insulin). For example, microvascular complications involving the retina, kidneys, and nerves are a major cause of morbidity and mortality in patients with type 1 diabetes or end-phase type 2 diabetes but are generally considered not prominent in patients that are resistant to insulin. There is increasing support for the concept that C-peptide deficiency may play a role in the development of the long-term complications of insulin-requiring diabetic patients. Additionally, in vivo as well as in vitro studies in diabetic human models and in patients with type 1 diabetes demonstrate that C-peptide possesses hormonal activity (Wahren J et al.: American Journal of Physiology 278: E759-E768, (2000); Wahren J et al.: In International Textbook of Diabetes Mellitus Ferranninni E, Zimmet P, De Fronzo R A, Keen H, Eds. John Wiley & Sons, (2004), p. 165-182).

### BRIEF SUMMARY

Surprisingly, the disclosure shows that so-called inflammation in metabolic syndrome in humans is augmented by a hitherto overlooked lock-and-key activation of the human elastin receptor, a protein involved in vascular (blood vessel) and elastin repair, with the C-peptide, a small protein that is produced in a 1:1 ratio alongside with widely known insulin. The elastin receptor is the lock that is activated by a key motif of amino acids (PG-domain) found in C-peptide and in breakdown products (PG-domain-fragments) thereof. Until now, no one has discovered this lock-and-key interaction between the two, now providing novel inroads in development of novel peptides and the use of peptides for treatment of metabolic syndrome, exploiting the finding that not only the normal keys of the elastin receptor (elastin peptides), but also the C-peptide, a peptide produced together with insulin every time glucose rises in our blood after a meal, interacts in a lock-and-key mode (docks) with the elastin receptor. Provided is isolated and synthetic peptides capable of modulating (mimicking, agonizing or antagonizing) binding of human C-peptide to a human elastin receptor for use in treatment of human disease.

Provided is an isolated or synthetic peptide for use in treatment of human disease, such as in treatment of human inflammation, and/or in treatment of type 1 diabetes and/or end-stage type 2 diabetes, more preferably use in treatment of micro-vascular complications, preferably as seen with type 1 diabetes and/or end-stage type 2 diabetes, wherein the peptide has at least one human elastin receptor binding motif GxxPG and has at least one amino acid Q, wherein G represents the one-letter code for the amino acid glycine, P for the amino acid proline, Q for the amino acid glutamine and x for any amino acid, the peptide consisting of 5-30 amino acids. Typically preferred peptides provided herein are selected from the group peptides listed under SEQ ID NOs: 10, 11, 12, 13, 17, 18, 19, 20, 14, 15, 16, 21, 25, 175, 3, 22, 23, 26, 176, 24, 27, 28, 29, 43, 93, and 94, and retro-inverso variant peptides derived from peptides listed under SEQ ID NOs: 11, 12, 13, 17, 18, 19, 20, 14, 15, 16, 21, 25, 175, 3, 22, 23, 26, 176, 24, 27, 28, 29, 43, 93, and 94. Note: Retro-inverse peptides are composed of D-amino acids assembled in a reverse order from that of the parent L-sequence, thus maintaining the overall topology of the native sequence. No stereoisomers of glycine exist, here (and in retro-inverso peptides bearing, for example, retro-inverso GxxP or xGxP motifs) G is not, whereas other amino acids, such as L, P and A are, instrumental to the all-D-amino acid character of the retro-inverso peptide herein provided. Provided is synthetic peptides wherein the human elastin receptor binding motif GxxPG motif has been repeated at least twice, preferably thrice, optionally said, repeats are separated by a linker, such a linker may comprise one or more amino acids, such as one or more amino acids selected from the group of glycine, alanine, leucine, valine, isoleucine or glutamine. In a preferred embodiment, provided is a peptide capable of combining with a human elastin receptor on a cell and initiating the same physiological activity typically produced by the binding of human C-peptide to the human elastin receptor. Provided is such an isolated or synthetic peptide for use in treatment of human disease, such as in treatment of human inflammation, and/or in treatment of type 1 diabetes and/or end-stage type 2 diabetes, more preferably use in treatment of micro-vascular complications, preferably as seen with type 1 diabetes and/or end-stage type 2 diabetes, wherein the peptide has at least one human elastin receptor binding motif GxxPG and has at least one amino acid Q, wherein G represents the one-letter code for the amino acid glycine, P for the amino acid proline, Q for the amino acid glutamine and x for any amino acid, the peptide consisting of 5-20 amino acids. Typically preferred peptides provided herein are selected from the group peptides listed under SEQ ID NOs: 14, 15, 16, 21, 25, 175, 3, 22, 23, 26, 176, 24, 27, 28, 29, 43, 93, and 94, and retro-inverso variant peptides derived from peptides listed under SEQ ID NOs: 14, 15, 16, 21, 25, 175, 3, 22, 23, 26, 176, 24, 27, 28, 29, 43, 93, and 94.

In a preferred embodiment, provided is a peptide capable of combining with a human elastin receptor on a cell and initiating the same physiological activity typically produced by the binding of human C-peptide to the human elastin receptor. Provided is such an isolated or synthetic peptide for use in treatment of human disease, such as in treatment of human inflammation, and/or in treatment of type 1 diabetes and/or end-stage type 2 diabetes, more preferably use in treatment of micro-vascular complications, preferably as seen with type 1 diabetes and/or end-stage type 2 diabetes, wherein the peptide has at least one human elastin receptor binding motif GxxPG and has at least one amino acid Q, wherein G represents the one-letter code for the amino acid glycine, P for the amino acid proline, Q for the amino acid glutamine and x for any amino acid, the peptide consisting of 5-15 amino acids. Typically preferred peptides provided herein are selected from the group peptides listed under SEQ ID NOs: 3, 22, 23, 26, 176, 24, 27, 28, 29, 43, 93, and 94, and retro-inverso variant peptides derived from peptides listed under SEQ ID NOs: 3, 22, 23, 26, 176, 24, 27, 28, 29, 43, 93, and 94.

In a preferred embodiment, provided is a peptide capable of combining with a human elastin receptor on a cell and initiating the same physiological activity typically produced by the binding of human C-peptide to the human elastin receptor. Provided is such an isolated or synthetic peptide for use in treatment of human disease, such as in treatment of human inflammation, and/or in treatment of type 1 diabetes and/or end-stage type 2 diabetes, more preferably use in treatment of micro-vascular complications, preferably as seen with type 1 diabetes and/or end-stage type 2 diabetes, wherein the peptide has at least one human elastin receptor binding motif GxxPG and has at least one amino acid Q, wherein G represents the one-letter code for the amino acid glycine, P for the amino acid proline, Q for the amino acid glutamine and x for any amino acid, the peptide consisting of 5-12 amino acids. Typically preferred peptides provided herein are selected from the group peptides listed under SEQ ID NOs: 24, 27, 28, 29, 43, 93, and 94, and retro-inverso variant peptides derived from peptides listed under SEQ ID NOs: 24, 27, 28, 29, 43, 93, and 94.

In a preferred embodiment, provided is a peptide capable of combining with a human elastin receptor on a cell and initiating the same physiological activity typically produced by the binding of human C-peptide to the human elastin receptor. Provided is such an isolated or synthetic peptide for use in treatment of human disease, such as in treatment of human inflammation, and/or in treatment of type 1 diabetes and/or end-stage type 2 diabetes, more preferably use in treatment of micro-vascular complications, preferably as seen with type 1 diabetes and/or end-stage type 2 diabetes, wherein the peptide has at least one human elastin receptor binding motif GxxPG and has at least one amino acid Q, wherein G represents the one-letter code for the amino acid glycine, P for the amino acid proline, Q for the amino acid glutamine and x for any amino acid, the peptide consisting of 5-9 amino acids. Typically preferred peptides provided are selected from the group peptides listed under SEQ ID NOs: 29, 43, 93, and 94, and retro-inverso variant peptides derived from peptides listed under SEQ ID NOs: 29, 43, 93, and 94.

In another preferred embodiment, provided is a peptide capable of inhibiting (inhibits) the binding of human C-peptide through C-peptide's motif GxxPG to the human elastin receptor. In a more preferred embodiment, provided is a peptide capable of reducing (reduces) the physiological activity of human C-peptide. In particular, provided is an isolated or synthetic peptide having at least the motif QDEA (SEQ ID NO:31) for use in treatment of human disease, such as in treatment of human insulin resistance and/or treatment of human dyslipidemia, and/or human hypertension, and/or human macrovascular complications, preferably complications seen in arteriosclerosis, atherosclerosis, peripheral arterial disease and/or new-onset type 2 diabetes, wherein the peptide inhibits the binding of human C-peptide to the human elastin receptor and reduces the physiological activity of human C-peptide, the peptide consisting of 4-40 amino acids. Typically preferred peptides provided herein are selected from the group peptides listed under SEQ ID NOs: 99, 100, 101, 131, 102, 103, 104, 105, 31, and functional fragments or variants thereof and retro-inverso variant peptides derived from peptides listed under SEQ ID NOs: 99, 100, 101, 131, 102, 103, 104, 105, 31, and functional fragments or variants thereof. Functional fragments or variants are typically found in chemotaxis assay as provided herein testing the capacity of peptides to inhibit binding of human C-peptide through C-peptide's motif GxxPG to the human elastin receptor, such a peptide capable of reducing (reduces) chemotaxis activity of human C-peptide.

In another embodiment, provided is a peptide capable of inhibiting (inhibits) the binding of human C-peptide through C-peptide's motif GxxPG to the human elastin receptor. In a more preferred embodiment, provided is a peptide capable of reducing (reduces) the physiological activity of human C-peptide. In particular, provided is an isolated or synthetic peptide having at least the motif QDEA (SEQ ID NO:31) for use in treatment of human disease, such as in treatment of human insulin resistance and/or treatment of human dyslipidemia, and/or human hypertension, and/or human macrovascular complications, preferably complications seen in arteriosclerosis, atherosclerosis, peripheral arterial disease and/or new-onset type 2 diabetes, wherein the peptide inhibits the binding of human C-peptide to the human elastin receptor and reduces the physiological activity of human C-peptide, the peptide consisting of 4-20 amino acids. Typically preferred peptides provided herein are selected from the group peptides listed under SEQ ID NOs: 131, 102, 103, 104, 105, 31, and functional fragments or variants thereof and retro-inverso variant peptides derived from peptides listed under SEQ ID NOs: 131, 102, 103, 104, 105, 31, and functional fragments or variants thereof.

In another embodiment, provided is a peptide capable of inhibiting (inhibits) the binding of human C-peptide through C-peptide's motif GxxPG to the human elastin receptor. In a more preferred embodiment, provided is a peptide capable of reducing (reduces) the physiological activity of human C-peptide. In particular, provided is an isolated or synthetic peptide having at least the motif QDEA (SEQ ID NO:31) for use in treatment of human disease, such as in treatment of human insulin resistance and/or treatment of human dyslipidemia, and/or human hypertension, and/or human macrovascular complications, preferably complications seen in arteriosclerosis, atherosclerosis, peripheral arterial disease and/or new-onset type 2 diabetes, wherein the peptide inhibits the binding of human C-peptide to the human elastin receptor and reduces the physiological activity of human C-peptide, the peptide consisting of 4-15 amino acids. Typically preferred peptides provided herein are selected from the group of peptides listed under SEQ ID NOs: 103, 104, 105, 31, and functional fragments or variants thereof and retro-inverso variant peptides derived from peptides listed under SEQ ID NOs: 103, 104, 105, 31, and functional fragments or variants thereof.

In another embodiment, provided is a peptide capable of inhibiting (inhibits) the binding of human C-peptide through C-peptide's motif GxxPG to the human elastin receptor. In a more preferred embodiment, provided is a peptide capable of reducing (reduces) the physiological activity of human C-peptide. In particular, provided is an isolated or synthetic peptide having at least the motif QDEA (SEQ ID NO:31) for use in treatment of human disease, such as in treatment of human insulin resistance and/or treatment of human dyslipidemia, and/or human hypertension, and/or human macrovascular complications, preferably complications seen in arteriosclerosis, atherosclerosis, peripheral arterial disease and/or new-onset type 2 diabetes, wherein the peptide inhibits the binding of human C-peptide to the human elastin receptor and reduces the physiological activity of human C-peptide, the peptide consisting of 4-9 amino acids. Typically preferred peptides provided herein are selected from the group peptides listed under SEQ ID NOs: 105, 31, and functional fragments or variants thereof and retro-inverso variant peptides derived from peptides listed under SEQ ID NOs: 105, 31, and functional fragments or variants thereof.

The disclosure relates and is limited to the use of peptide agonists and/or peptide antagonists of human C-peptide's interaction with the human elastin receptor for treatment of human disease. Therewith, provided is three fields of use of peptides in human disease.

A first field of the disclosure, named Drug: anti-inflammatory peptides, means any peptide for use as a drug to counteract (control, reduce or treat) human inflammatory disease or to any peptide component of a drug to counteract human inflammatory disease or to any peptide used in the preparation of a drug to counteract human inflammatory disease (counteract in this description also generally identified as treat or use in treatment), wherein the peptide has at least one human elastin receptor binding motif GxxP, or its functionally equivalent xGxP, has at least one amino acid Q, and wherein G represents the one-letter code for the amino acid glycine, P for the amino acid proline, Q for the amino acid glutamine and x for any amino acid. In some embodiments, the Q may be replaced by a functionally equivalent L (leucine). Preferably, the peptide of this first field has at least one human elastin receptor binding motif xGxxPG. Such peptides are useful in the treatment of inflammatory conditions, such as acute kidney injury, also in acute systemic inflammatory conditions such as, for example, sepsis or systemic inflammatory response syndrome (SIRS), leading to vascular damage and often aggravated by (multiple organ) organ failure, or inflammatory conditions with diabetes, when given with an anti-diabetic composition such as insulin.

A second field of the disclosure, named Drug: C-peptide agonists, means any agonist peptide for use as a medicine to treat human microvascular complications or any agonist peptide component of a medicine to treat human microvascular complications or any agonist peptide used in the preparation of a medicine to treat human microvascular complications, wherein the peptide has at least one human elastin receptor binding motif GxxP, or its functionally equivalent xGxP, has at least one amino acid Q, wherein G stands for the amino acid glycine, P stands for the amino acid proline, Q stands for the amino acid glutamine and x stands for any amino acid, and is capable of combining with a human elastin receptor on a cell and initiating the same physiological activity typically produced by the binding of human C-peptide to the human elastin receptor. In some embodiments, the Q may be replaced by a functionally equivalent L (leucine). Preferably, the peptide of this second field has at least one human elastin receptor binding motif xGxxPG. Such peptides are useful in the treatment of inflammatory conditions, such as acute kidney injury, also in acute systemic inflammatory conditions such as, for example, sepsis or systemic inflammatory response syndrome (SIRS), leading to vascular damage and often aggravated by (multiple organ) organ failure, or inflammatory conditions with diabetes, when given with an anti-diabetic composition such as insulin.

A third field of the disclosure, named Drug: C-peptide antagonists, means any antagonist peptide for use as a medicine to treat human insulin resistance, dyslipidemia, hypertension or macrovascular complications or to any antagonist peptide component of a medicine to treat human insulin resistance, dyslipidemia, hypertension or macrovascular complications or to any antagonist peptide used in the preparation of a medicine to treat human insulin resistance, dyslipidemia, hypertension or macrovascular complications, wherein the antagonist peptide inhibits the binding of human C-peptide through its motif GxxP to the human elastin receptor and reduces the physiological activity of human C-peptide.

Herein, a peptide or peptide fragment having a PG-domain is particularly defined as a peptide having at least one xGxP, GxxP, GxxPG or xGxPG motif, G being Glycine, P being Proline, x being any amino acid, the amino acid following P preferably allowing for a type VIII-beta turn, a condition that is met when P is C-terminally followed by a G. The present disclosure provides a method for preventing or treating disease comprising providing a human with a peptide capable of agonizing an elastin receptor. Such peptides as herein provided are preferably selected from the group of fragments of C-peptide and functional equivalents thereof.

In a first embodiment, provided is a peptide for use in the treatment of human inflammation, preferably of a human subject in need thereof, in particularly in a human subject having been diagnosed as suffering from type 1 diabetes, most preferably when the subject is also treated with insulin, wherein the peptide is selected from the group of, preferably isolated and/or synthetic, preferably non-PEGylated, C-peptide fragments human 1-24 SEQ ID NO:17 (EAEDLQVGQVELGGGPGAGSLQPL), human 4-24 SEQ ID NO:20 (DLQVGQVELGGGPGAGSLQPL), human 7-24 SEQ ID NO:175 (VGQVELGGGPGAGSLQPL), human 11-24 SEQ ID NO:176 (ELGGGPGAGSLQPL), human 4-31 SEQ ID NO:10 (DLQVGQVELGGGPGAGSLQPLALEGSLQ), human 8-31 SEQ ID NO:13 (GQVELGGGPGAGSLQPLALEGSLQ) and human 12-31 SEQ ID NO:14 (LGGGPGAGSLQPLALEGSLQ), as listed in FIG. 1 in this disclosure, the peptide showing the GxxP motif SEQ ID NO:38 (GGGP), and a significant normalization (%) of 30 mM glucose-induced vascular dysfunction in rats.

Also, a peptide with motif SEQ ID NO:38 (GGGP), functionally equivalent to a peptide listed in FIG. 1 herein, for use in the treatment of human inflammation, in particularly in a human subject having been diagnosed as suffering from type 1 diabetes, most preferably when the subject is also treated with insulin, as provided herein is the synthetic and isolated peptide SEQ ID NO:10 (DLQVGQVELGGGPGAGSLQPLALEGSLQ) as derivable from the human C-peptide sequence. Also, a peptide with motif SEQ ID NO:38 (GGGP), functionally equivalent to a peptide listed in FIG. 1 herein, for use in the treatment of human inflammation, in particularly in a human subject having been diagnosed as suffering from type 1 diabetes, most preferably when the subject is also treated with insulin, as provided herein is the synthetic and isolated peptide SEQ ID NO:11 (LQVGQVELGGGPGAGSLQPLALEGSLQ) as obtainable from the human C-peptide sequence. Also, a peptide with motif SEQ ID NO:38 (GGGP), functionally equivalent to a peptide listed in FIG. 1 herein, for use in the treatment of human inflammation, in particularly in a human subject having been diagnosed as suffering from type 1 diabetes, most preferably when the subject is also treated with insulin, most preferably when the subject is also treated with insulin, as provided herein is the synthetic and isolated peptide SEQ ID NO:12 (VGQVELGGGPGAGSLQPLALEGSLQ) as derivable from the human C-peptide sequence. Also, a peptide with motif SEQ ID NO:38 (GGGP), functionally equivalent to a peptide listed in FIG. 1 herein, for use in the treatment of human inflammation, in particularly in a human subject having been diagnosed as suffering from type 1 diabetes, most preferably when the subject is also treated with insulin, as provided herein is the synthetic and isolated peptide SEQ ID NO:13 (GQVELGGGPGAGSLQPLALEGSLQ) as derivable from the human C-peptide sequence. Also, a peptide with motif SEQ ID NO:38 (GGGP), functionally equivalent to a peptide listed in FIG. 1 herein, for use in the treatment of human inflammation, in particularly in a human subject having been diagnosed as suffering from type 1 diabetes, most preferably when the subject is also treated with insulin, as provided herein is the synthetic and isolated peptide SEQ ID NO:14 (LGGGPGAGSLQPLALEGSLQ) as derivable from the human C-peptide sequence. Also, a peptide with motif SEQ ID NO:38 (GGGP), functionally equivalent to a peptide listed in FIG. 1 herein, for use in the treatment of human inflammation, in particularly in a human subject having been diagnosed as suffering from type 1 diabetes, most preferably when the subject is also treated with insulin, as provided herein is the synthetic and isolated peptide SEQ ID NO:15 (VGQVELGGGPGAGSLQPLAL) as derivable from the human C-peptide sequence. Also, a peptide with motif SEQ ID NO:38 (GGGP), functionally equivalent to a peptide listed in FIG. 1 herein, for use in the treatment of human inflammation, in particularly in a human subject having been diagnosed as suffering from type 1 diabetes, most preferably when the subject is also treated with insulin, as provided herein is the synthetic and isolated peptide SEQ ID NO:16 (EVGQVELGGGPGAGSLQPL) as derivable from the human C-peptide sequence. In another embodiment, a peptide with motif SEQ ID NO:38 (GGGP), functionally equivalent to a peptide listed in FIG. 1 herein, is provided for use in the treatment of human inflammation, in particularly in a human subject having been diagnosed as suffering from type 1 diabetes, most preferably when the subject is also treated with insulin, as provided herein is the synthetic and isolated peptide SEQ ID NO:17 (EAEDLQVGQVELGGGPGAGSLQPLAL) as derivable from the human C-peptide sequence. In another embodiment, a peptide with motif SEQ ID NO:38 (GGGP), functionally equivalent to a peptide listed in FIG. 1 herein, for use in the treatment of human inflammation, in particularly in a human subject having been diagnosed as suffering from type 1 diabetes, most preferably when the subject is also treated with insulin, as provided herein is the synthetic and isolated peptide SEQ ID NO:18 (EAEDLQVGQVELGGGPGAGSLQPL) as derivable from the human C-peptide sequence. In another embodiment, a peptide with motif SEQ ID NO:38 (GGGP), functionally equivalent to a peptide listed in FIG. 1 herein, for use in the treatment of human inflammation, in particularly in a human subject having been diagnosed as suffering from type 1 diabetes, most preferably when the subject is also treated with insulin, as provided herein is the synthetic and isolated peptide SEQ ID NO:19 (LQVGQVELGGGPGAGSLQPLAL) as derivable from the human C-peptide sequence. In another embodiment, a peptide with motif SEQ ID NO:38 (GGGP), functionally equivalent to a peptide listed in FIG. 1 herein, for use in the treatment of human inflammation, in particularly in a human subject having been diagnosed as suffering from type 1 diabetes, most preferably when the subject is also treated with insulin, as provided herein is the synthetic and isolated peptide SEQ ID NO:20 (DLQVGQVELGGGPGAGSLQPL) as derivable from the human C-peptide sequence. In another embodiment, a peptide with motif SEQ ID NO:38 (GGGP), functionally equivalent to a peptide listed in FIG. 1 herein, is provided for use in the treatment of human inflammation, in particularly in a human subject having been diagnosed as suffering from type 1 diabetes, most preferably when the subject is also treated with insulin, as provided herein is the synthetic and isolated peptide SEQ ID NO:21 (LQVGQVELGGGPGAGSLQPL) as derivable from the human C-peptide sequence. In another embodiment, a peptide with motif SEQ ID NO:38 (GGGP), functionally equivalent to a peptide listed in FIG. 1 herein, for use in the treatment of human inflammation, in particularly in a human subject having been diagnosed as suffering from type 1 diabetes, most preferably when the subject is also treated with insulin, as provided herein is the synthetic and isolated peptide SEQ ID NO:22 (LGGGPGAGSLQPL) as derivable from the human C-peptide sequence. In another embodiment, a peptide with motif SEQ ID NO:38 (GGGP), functionally equivalent to a peptide listed in FIG. 1 herein, for use in the treatment of human inflammation, in particularly in a human subject having been diagnosed as suffering from type 1 diabetes, most preferably when the subject is also treated with insulin, as provided herein is the synthetic and isolated peptide SEQ ID NO:23 (VGQVELGGGPGAGSL) as derivable from the human C-peptide sequence. In another embodiment, a peptide with motif SEQ ID NO:38 (GGGP), functionally equivalent to a peptide listed in FIG. 1 herein, for use in the treatment of human inflammation, in particularly in a human subject having been diagnosed as suffering from type 1 diabetes, most preferably when the subject is also treated with insulin, as provided herein is the synthetic and isolated peptide SEQ ID NO:24 (GGGPGAGSLQ) as derivable from the human C-peptide sequence.

Provided is an isolated and/or synthetic, preferably non-PEGylated, peptide identified herein as a regulatory model element peptide or fragment thereof that it is identified herein in specific regulatory elements modulating inflammation and tissue repair. Provided is an isolated and/or synthetic peptide wherein the regulatory model element peptide or fragment preferably carries a xGxxPG or xxGxPG motif and preferably can be derived, for example, from proteins identified in Table 3 herein. In a preferred embodiment, the model element is recognized while it is flanked by at least one N-terminal and at least one C-terminal basic amino acid residue R (arginine) or K (lysine). Smaller fragments from within the element not carrying the two flanking basic residues are also useful in modulating inflammation and tissue repair. In one embodiment, a regulatory model element peptide fragment for use in the treatment of human inflammation as provided herein is the synthetic and isolated peptide SEQ ID NO:25 (FRAAPLQGMLPGLLAPLRT) as derivable from the human COL6A3 sequence (in UIniprot database COL6A3 is known under identifier P12111 and the SEQ ID NO:25 (FRAAPLQGMLPGLLAPLRT) sequence is found in the sequence in isoform 1 from at around position 605-626). It is herein provided that the peptide is useful in the treatment of human inflammation and/or tissue repair, as are fragments thereof. In another embodiment, a regulatory model element peptide fragment for use in the treatment of human inflammation and/or tissue repair as provided herein is the synthetic and isolated peptide SEQ ID NO:26 (AAPLQGMLPGLLAPL) as derivable from the human COL6A3 sequence. In another embodiment, a regulatory model element peptide fragment for use in the treatment of human inflammation and/or tissue repair as provided herein is the synthetic and isolated peptide SEQ ID NO:27 (LQGMLPGLLAPL) as derivable from the human COL6A3 sequence. In another embodiment, a regulatory model element peptide fragment for use in the treatment of human inflammation and/or tissue repair as provided herein is the synthetic and isolated peptide SEQ ID NO:28 (LQGMLPGLLA) as derivable from the human COL6A3 sequence. In another embodiment, a regulatory model element peptide fragment for use in the treatment of human inflammation and/or tissue repair as provided herein is the synthetic and isolated peptide SEQ ID NO:29 (LQGMLPG) as derivable from the human COL6A3 sequence. In another embodiment, a regulatory model element peptide fragment for use in the treatment of human inflammation and/or tissue repair as provided herein is the synthetic and isolated peptide SEQ ID NO:30 (GMLPGLLA) as derivable from the human COL6A3 sequence.

In another embodiment, a regulatory model element peptide fragment for use in the treatment of human inflammation and/or tissue repair as provided herein is the synthetic and isolated peptide SEQ ID NO:177 (CGNLSTCMLGTYTQDFNKFHTFPQTAIGVGAPG) as derivable from the human procalcitonin sequence.

In another embodiment, a regulatory model element peptide fragment for use in the treatment of human inflammation and/or tissue repair as provided herein is the synthetic and isolated peptide SEQ ID NO:178 (MLGTYTQDFNKFHTFPQTAIGVGAPG) as derivable from the human procalcitonin sequence.

In another embodiment, a regulatory model element peptide fragment for use in the treatment of human inflammation and/or tissue repair as provided herein is the synthetic and isolated peptide SEQ ID NO:179 (FNKFHTFPQTAIGVGAPG) as derivable from the human procalcitonin sequence. In another embodiment, a regulatory model element peptide fragment for use in the treatment of human inflammation and/or tissue repair as provided herein is the synthetic and isolated peptide SEQ ID NO:180 (FPQTAIGVGAPG) as derivable from the human procalcitonin sequence. In another embodiment, a regulatory model element peptide fragment for use in the treatment of human inflammation and/or tissue repair as provided herein is the synthetic and isolated peptide SEQ ID NO:181 (AIGVGAPG) as derivable from the human procalcitonin sequence.

In another embodiment, a regulatory model element peptide fragment for use in the treatment of human inflammation and/or tissue repair as provided herein is the synthetic and isolated peptide SEQ ID NO:182 (SHPLGSPGSASDLETSGLQEQ) as derivable from the human NTproBNP sequence. In another embodiment, a regulatory model element peptide fragment for use in the treatment of human inflammation and/or tissue repair as provided herein is the synthetic and isolated peptide SEQ ID NO:183 (PLGSPGSASDLETSGLQEQ) as derivable from the human NTproBNP sequence. In another embodiment, a regulatory model element peptide fragment for use in the treatment of human inflammation and/or tissue repair as provided herein is the synthetic and isolated peptide SEQ ID NO:184 (PLGSPGSASDLETS) as derivable from the human NTproBNP sequence. In another embodiment, a regulatory model element peptide fragment for use in the treatment of human inflammation and/or tissue repair as provided herein is the synthetic and isolated peptide SEQ ID NO:185 (PLGSPGSAS) as derivable from the human NTproBNP sequence. In another embodiment, a regulatory model element peptide fragment for use in the treatment of human inflammation and/or tissue repair as provided herein is the synthetic and isolated peptide SEQ ID NO:186 (PLGSPG) as derivable from the human NTproBNP sequence. In another embodiment, a regulatory model element peptide fragment for use in the treatment of human inflammation and/or tissue repair as provided herein is the synthetic and isolated peptide SEQ ID NO:187 (EDVSAGEDCGPLPEGGPEPRSDGAKPGPREG) as derivable from the human POMC sequence. In another embodiment, a regulatory model element peptide fragment for use in the treatment of human inflammation and/or tissue repair is the synthetic and isolated peptide SEQ ID NO:188 (GEDCGPLPEGGPEPRSDGAKPGPREG) as derivable from the human POMC sequence. In another embodiment, a regulatory model element peptide fragment for use in the treatment of human inflammation and/or tissue repair is the synthetic and isolated peptide SEQ ID NO:189 (PLPEGGPEPRSDGAKPGPREG) as derivable from the human POMC sequence. In another embodiment, a regulatory model element peptide fragment for use in the treatment of human inflammation and/or tissue repair is the synthetic and isolated peptide SEQ ID NO:190 (PLPEGGPEPRSDGAKPG) as derivable from the human POMC sequence. In another embodiment, a regulatory model element peptide fragment for use in the treatment of human inflammation and/or tissue repair is the synthetic and isolated peptide SEQ ID NO:191 (SDGAKPG) as derivable from the human POMC sequence.

In another embodiment, a regulatory model element peptide fragment for use in the treatment of human inflammation and/or tissue repair as provided herein is the synthetic and isolated peptide SEQ ID NO:192 (RRNASSAGRLQGLAGGAPGQKECR) as derivable from the human pyrin sequence. It is herein provided that the peptide is useful in the treatment of human inflammation and/or tissue repair, as are fragments thereof. In another embodiment, a regulatory model element peptide fragment for use in the treatment of human inflammation and/or tissue repair as provided herein is the synthetic and isolated peptide SEQ ID NO:193 (RLQGLAGGAPGQKECR) as derivable from the human pyrin sequence. In another embodiment, a regulatory model element peptide fragment for use in the treatment of human inflammation and/or tissue repair as provided herein is the synthetic and isolated peptide SEQ ID NO:194 (RRNASSAGRLQGLAGGAPGQ) as derivable from the human pyrin (marenostrin) sequence.

In another embodiment, a regulatory model element peptide fragment for use in the treatment of human inflammation and/or tissue repair as provided herein is the synthetic and isolated peptide SEQ ID NO:195 (LQGLAGGAPGQ) as derivable from the human pyrin sequence. In another embodiment, a regulatory model element peptide fragment for use in the treatment of human inflammation and/or tissue repair as provided herein is the synthetic and isolated peptide SEQ ID NO:196 (AGGAPG) as derivable from the human pyrin sequence. Provided is use in humans of a peptide consisting of 4-40 amino acids, preferably of 4-20, more preferably of 4-15, more preferably 4-12, most preferably of 4-9 amino acids, the peptide comprising at least one PG-domain, preferably with a xGxP or GxxP, GxxPG or xGxPG motif.

Provided is a method for preventing or treating disease comprising providing a human with a peptide capable of antagonizing, blocking, inhibiting or preventing of binding of fragments of C-peptide and/or of elastin peptide to an elastin receptor. Provided is a method wherein the peptide or fragments comprise a binding site allowing binding to an elastin receptor. Provided is a method wherein the binding site comprises an amino acid sequence motif GxxP, allowing a type VIII beta-turn. Provided is a method wherein the binding site comprises an amino acid sequence motif xGxPG or GxxPG. Provided is a method wherein the binding site comprises an amino acid sequence motif xGxP or GxxP, the P allowing a type VIII beta-turn. Provided is a method wherein the binding site comprises an amino acid sequence motif GxxPG.

Provided is a method wherein the peptide fragments comprise a binding site allowing binding to an elastin receptor. Provided is a method wherein the binding site comprises an amino acid sequence motif xGxP or GxxP, allowing a type VIII beta-turn.

Provided is a method wherein the binding site comprises an amino acid sequence motif xGxPG or GxxPG.

Provided is use in a human of a composition wherein the motif allows the peptide to modulate binding of C-peptide to an elastin receptor. Provided is use in a human of a pharmaceutical composition comprising a peptide consisting of 4-40 amino acids, preferably of 4-20, more preferably of 4-15, more preferably 4-12, most preferably of 4-9 amino acids, the peptide comprising the motif SEQ ID NO:31 (QDEA).

Provided is method for preventing or treating disease of a human comprising providing the human with a peptide capable of antagonizing, blocking, inhibiting or preventing of binding of fragments of C-peptide and/or of elastin peptide to an elastin receptor of the human. In an embodiment of a method for preventing or treating disease of a human. It is most preferred that the peptide or fragments comprise an amino acid motif allowing binding to an elastin receptor.

Provided is a method for preventing or treating disease of a human comprising providing the human with a peptide capable of agonizing an elastin receptor of the human.

Provided is a use in a human of a composition, preferably of a pharmaceutical composition or medicament, comprising a peptide consisting of 4-40 amino acids, preferably of 4-more preferably of 4-15, more preferably 4-12, most preferably of 4-9 amino acids, the peptide comprising at least one xGxP, GxxP, GxxPG or xGxPG motif, the motif preferably allowing the peptide to modulate binding of C-peptide to an elastin receptor. In a preferred embodiment, the composition is prepared for the treatment of diabetes, preferably for the treatment or prevention of microvascular disorders seen with diabetes wherein the endogenous C-peptide level is low, such as with type 1 diabetes or with end-stage type 2 diabetes in a human.

Provided is use of a peptide consisting of 4-40 amino acids, preferably of 4-20, more preferably of 4-15, more preferably 4-12, most preferably of 4-9 amino acids, the peptide comprising at least one xGxP, GxxP, GxxPG or xGxPG motif, the motif preferably allowing the peptide to modulate binding of C-peptide to an elastin receptor, for the production of a medicament, preferably of a medicament for the treatment or prevention of microvascular disorders seen with diabetes wherein the endogenous C-peptide level is low, such as with type 1 diabetes or with end-stage type 2 diabetes.

Provided is use in a human of a composition, preferably a pharmaceutical composition or medicament, comprising a peptide consisting of 4-40 amino acids, preferably of 4-more preferably of 4-15, more preferably 4-12, most preferably of 4-9 amino acids, the peptide comprising the motif SEQ ID NO:31 (QDEA), preferably the motif allowing the peptide to modulate binding of C-peptide to an elastin receptor, preferably for the treatment or prevention of conditions of metabolic syndrome as defined herein, preferably for the treatment or prevention of cardiovascular disease or macrovascular disease or atheromatous disease such as atherosclerosis or arteriosclerosis.

Provided is use in a human of a peptide consisting of 4-40 amino acids, preferably of 4-20, more preferably of 4-15, more preferably 4-12, most preferably of 4-9 amino acids, the peptide comprising the motif SEQ ID NO:31 (QDEA), preferably the motif allowing the peptide to modulate binding of C-peptide to an elastin receptor, for the treatment or prevention of conditions of metabolic syndrome as defined herein, preferably for the treatment or prevention of cardiovascular disease or macrovascular disease or atheromatous disease such as atherosclerosis or arteriosclerosis.

The disclosure shows that the so-called inflammation in metabolic syndrome is augmented by an hitherto overlooked lock-and-key activation of the elastin receptor, a protein involved in vascular (blood vessel) elastin repair, with the C-peptide, a small protein that is produced in a 1:1 ratio alongside with widely known insulin. The human elastin receptor is the lock that is activated by a key motif of amino acids (GxxP) found in C-peptide and in breakdown products (GxxP-fragments) thereof. Until now, no one has ever discovered this lock-and-key interaction between the two, now providing novel inroads in development of novel peptides for treatment of metabolic syndrome, exploiting the finding that not only the normal keys of the elastin receptor (elastin peptides), but also the C-peptide, a peptide produced together with insulin every time glucose rises in our blood after a meal, interacts in a lock-and-key mode with the elastin receptor. In summary, provided is the insight that excess food intake directly switches C-peptide on as the key unlocking metabolic syndrome. Everyday overeating results in everyday increased C-peptide levels (and GxxP motif containing break-down fragments thereof) in the blood. As the elastin receptor is mainly found on cells that produce elastin and on cells that repair our blood vessels (together called vascular cells), everyday GxxP lock-and-key activation of the elastin receptor by excess C-peptide and its fragments results in everyday blood vessel damage done. As 30% of the walls of our blood-vessels are made up of elastin and inflammatory cells continuously repair damage done to blood vessels, disturbing elastin repair and provoking inflammation of blood vessels cannot remain without consequences. Indeed, continued elastin receptor activation by C-peptide and GxxP fragments leaves a state of vascular over-repair, hitherto called inflammation, and otherwise called atherosclerosis, a condition characterized by thickening of blood vessel walls with activated inflammatory and blood vessel cells that underlies all conditions of metabolic syndrome. Over years, and in trickling fashion that varies from person to person, more-and-more damage is done to the elasticity and strength of our vasculature of various organs (such as heart, blood vessels, pancreas, kidney, brain) that generally leads to atherosclerosis, hypertension and dyslipidemia, and ultimately leads to various manifestations as cardiovascular disease, diabetes type 2, chronic kidney failure and vascular dementias.

The finding explains how every day excess food intake results in over-repair of blood vessels, also called chronic inflammation. In short: every time we consume food with glucose (sugar) we produce insulin and thus C-peptide, every time we eat too much glucose, we produce more-and-more insulin, and thus more-and-more (excess) C-peptide. It is this excess C-peptide and the fragments thereof that still carry the key unlocking motif GxxP that cause excess elastin receptor activation, leading to vascular over-repair with inflammation. Overeating every day directly causes excess production of C-peptide and its GxxP-fragments that every day adds up to elastin receptor induced over-repair, leading up to the chronic over-repair and so-called inflammation, the dyslipidaemia, hypertension and ultimately unhealthy blood vessels seen in metabolic syndrome. With this insight, the finding provides roads to develop and use products (immunolatory peptides) to block GxxP lock-and-key interaction to prevent disease, including hypertension and atherosclerosis. Also, the disclosure explains the added risks of a sedentary live. In short: not using our unhealthy intake of sugary food as fuel for our muscles urges our bodies to produce more-and-more insulin to help the liver to change the excess sugar into fat that can comes back in the blood leaving us with dyslipidemia. Again, excess C-peptide is produced along with insulin, again setting the lock-and-key elastin receptor activation in motion, and leading up to the deregulation of fat metabolism as described. Thirdly, the disclosure explains the added risks of smoking as well. In short: smoking (or similarly: air pollution) causes damage to the elastic tissue of the lung, thereby releasing fragments of elastin having the GxxP motif (herein called elastin peptides) that are known to cause elastin receptor activation. Thus, smoking adds more peptides with the GxxP motif to the already circulating C-peptide fragments with that motif. This accumulation of diet-induced C-peptide and smoking-induced elastin peptide ads up to aggravated over-repair and so-called inflammation and thus aggravated cardiovascular or chronic kidney disease in those people that both smoke and indulge in too much sugar from their diet. The disclosure is showing an as yet fully unknown common causal relationship between diseases caused by different lifestyle conditions, overeating, being sedentary and smoking. Provided is peptides to block GxxP lock- and key interaction to prevent disease. Both C-peptide and elastin peptides, and their breakdown products or fragments are relatively stable in blood and urine. Where insulin is rapidly degraded in the liver and disappears from the blood, C-peptide (and breakdown fragments with the GxxP motif) as well as elastin peptides have a much longer life in the blood and are only excreted by the kidney. Thus, whether eating-on and producing new insulin with new C-peptide, or continuing smoking and producing more elastin decay of the lungs; levels of C-peptide and elastin peptides build up and over time cause more and more vascular damage via GxxP-mediated activation of the elastin receptor. GxxP lock-and key interaction of both C-peptide and elastin peptides may be blocked with appropriate peptides to prevent disease depending on the outcome of these diagnostic tests as provided herein. Also, provided is peptides that stimulate (agonists) or inhibit (antagonists) GxxP-receptor binding. Agonists may be used in patients wherein C-peptide levels are low, such as in people suffering from diabetes type 1 or end-phase diabetes type 2. Antagonists may be used in patients wherein C-peptide levels are high, in particular, in people suffering or prone to be suffering from metabolic syndrome.

Provided is a method of treating or preventing metabolic syndrome or insulin resistance or hypertension or atherosclerosis or dyslipidemia or diabetes type-2 or a related metabolic disorder in a human suffering therefrom or in need thereof, the method comprising: administering to the human an antagonist of the C-peptide/elastin binding protein interaction. In a further embodiment, the method further comprises administering insulin in the absence of C-peptide to the human. In another embodiment, provided is a method of treating or preventing metabolic syndrome or insulin resistance or hypertension or atherosclerosis or diabetes type-2 or metabolic disorder in a human suffering therefrom or in need thereof, the method comprising: administering to the human an antagonist of the C-peptide/elastin binding protein interaction, the method additionally comprising administering to the human an antagonist to alpha-enolase. In a further embodiment, the method further comprises administering insulin in the absence of C-peptide to the human. In yet another embodiment, provided is a method of treating or preventing metabolic syndrome or insulin resistance or hypertension or atherosclerosis or diabetes type-2 or metabolic disorder in a human suffering therefrom or in need thereof, the method comprising: administering to the human an antagonist of the C-peptide/elastin binding protein interaction, the method additionally comprising administering to the human an antagonist to GPR146. In a further embodiment, the method further comprises administering insulin in the absence of C-peptide to the human.

Provided is use in a human of an isolated fragment of a C-peptide as an agent that modulates binding or interaction of a C-peptide with an elastin receptor, and/or use of an isolated fragment of an elastin receptor as an agent that modulates binding or interaction of a C-peptide with an elastin receptor. Examples of such peptide (fragments or variants) comprise peptides such as, peptide comprising SEQ ID NO:32 (LGGGPGAG) or a fragment thereof, or SEQ ID NO:33 (LAGGPGAG) or a fragment thereof, or having SEQ ID NO:34 (LGGGPG) or SEQ ID NO:35 (LAGGPG) or a fragment thereof, preferably wherein the peptide or fragment is selected from the group consisting of SEQ ID NO:36 (LGGGP), SEQ ID NO:37 (LAGGP), SEQ ID NO:38 (GGGP), and SEQ ID NO:39 (GAGP), or retro-inverso variant peptide of SEQ ID NO:32 (LGGGPGAG) or a fragment thereof, or of SEQ ID NO:33 (LAGGPGAG) or a fragment thereof, or of SEQ ID NO:34 (LGGGPG) or SEQ ID NO:35 (LAGGPG) or a fragment thereof, preferably wherein the peptide or fragment is selected from the group consisting of retro-inverso variants of SEQ ID NO:36 (LGGGP), SEQ ID NO:37 (LAGGP), SEQ ID NO:38 (GGGP), as further discussed below.

The present disclosure also provides a peptide derived from a fragment of mammalian insulin C-peptide for use in human therapy, preferably for use in the treatment of human diabetes and/or human diabetic complications, or for reducing inflammatory activity. It is preferred that the peptide or fragment is from two (2) to nine (9) amino acids in length, more preferably three (3) to six (6) amino acids in length, most preferably from four (4) tot five (5) amino acids in length. Provided is a peptide derived from a fragment of mammalian insulin C-peptide, the peptide comprising SEQ ID NO:32 (LGGGPGAG) or a fragment thereof, or SEQ ID NO:33 (LAGGPGAG) or a fragment thereof, or having SEQ ID NO:34 (LGGGPG) or SEQ ID NO:35 (LAGGPG) or a fragment thereof, preferably wherein the peptide or fragment is selected from the group consisting of SEQ ID NO:36 (LGGGP), SEQ ID NO:37 (LAGGP), SEQ ID NO:38 (GGGP), and SEQ ID NO:39 (GAGP), the peptide having the ability to interact with elastin receptor type binding or modulate inflammatory activity of innate immune cells. It is preferred that the peptide or fragment is from two (2) to nine (9) amino acids in length, more preferably three (3) to six (6) amino acids in length, most preferably from four (4) tot five (5) amino acids in length. Provided is an isolated or synthetic peptide, essentially being homologous to a fragment of mammalian insulin C-peptide, the peptide comprising SEQ ID NO:32 (LGGGPGAG) or a fragment thereof, or SEQ ID NO:33 (LAGGPGAG) or a fragment thereof, or having SEQ ID NO:34 (LGGGPG) or SEQ ID NO:35 (LAGGPG) or a fragment thereof, preferably wherein the peptide or fragment is selected from the group consisting of SEQ ID NO:36 (LGGGP), SEQ ID NO:37 (LAGGP), SEQ ID NO:38 (GGGP), and SEQ ID NO:39 (GAGP), the peptide having the ability to interact with elastin receptor type binding or modulate inflammatory activity of innate immune cells. It is preferred that the peptide or fragment is from two (2) to nine (9) amino acids in length, more preferably three (3) to six (6) amino acids in length, most preferably from four (4) tot five (5) amino acids in length.

Provided is retro-inverso variants of SEQ ID NO:32 (LGGGPGAG) or a fragment thereof, or SEQ ID NO:33 (LAGGPGAG) or a fragment thereof, or of SEQ ID NO:34 (LGGGPG) or SEQ ID NO:35 (LAGGPG) or a fragment thereof, preferably wherein the peptide or fragment is selected from the group consisting of retro-inverso variants of SEQ ID NO:36 (LGGGP), SEQ ID NO:37 (LAGGP), SEQ ID NO:38 (GGGP) or SEQ ID NO:39 (GAGP). It is preferred that the retro-inverso variant peptide is from two (2) to nine (9) amino acids in length, more preferably three (3) to six (6) amino acids in length, most preferably from four (4) tot five (5) amino acids in length.

Provided is use in human of a peptide comprising SEQ ID NO:32 (LGGGPGAG) or a fragment thereof, or SEQ ID NO:33 (LAGGPGAG) or a fragment thereof, or having SEQ ID NO:34 (LGGGPG) or SEQ ID NO:35 (LAGGPG) or a fragment thereof, preferably wherein the peptide or fragment is selected from the group consisting of SEQ ID NO:36 (LGGGP), SEQ ID NO:37 (LAGGP), SEQ ID NO:38 (GGGP), and SEQ ID NO:39 (GAGP), the peptide or fragment having the ability to interact with elastin receptor type binding or modulate inflammatory activity of innate immune cells together with at least one pharmaceutically acceptable carrier or excipient. It is preferred that the peptide or fragment is from two (2) to nine (9) amino acids in length, more preferably three (3) to six (6) amino acids in length, most preferably from four (4) tot five (5) amino acids in length. In a preferred embodiment, the peptide is combined with at least one additional active agent effective to combat diabetes, diabetic complications, or to treat an inflammatory condition, such as insulin or metformin, and/or or wherein the additional active agent is an interleukin-1 receptor antagonist or an antibody directed against an interleukin-1, preferably directed against interleukin-1-beta, or an agonist of alpha-enolase or an agonist of GPR146.

Provided is use in human of a retro-inverso variant peptide of SEQ ID NO:32 (LGGGPGAG) or a fragment thereof, or of SEQ ID NO:33 (LAGGPGAG) or a fragment thereof, or of SEQ ID NO:34 (LGGGPG) or SEQ ID NO:35 (LAGGPG) or a fragment thereof, preferably wherein the peptide or fragment is selected from the group consisting of retro-inverso variants of SEQ ID NO:36 (LGGGP), SEQ ID NO:37 (LAGGP), SEQ ID NO:38 (GGGP), and SEQ ID NO:39 (GAGP), the peptide or fragment having the ability to interact with elastin receptor type binding or modulate inflammatory activity of innate immune cells together with at least one pharmaceutically acceptable carrier or excipient. It is preferred that the peptide or fragment is from two (2) to nine (9) amino acids in length, more preferably three (3) to six (6) amino acids in length, most preferably from four (4) tot five (5) amino acids in length. In a preferred embodiment, the peptide is combined with at least one additional active agent effective to combat diabetes, diabetic complications, or to treat an inflammatory condition, such as insulin or metformin, and/or or wherein the additional active agent is an interleukin-1 receptor antagonist or an antibody directed against an interleukin-1, preferably directed against interleukin-1-beta, or an agonist of alpha-enolase or an agonist of GPR146.

Provided is use of at least one peptide comprising SEQ ID NO:32 (LGGGPGAG) or a fragment thereof, or SEQ ID NO:33 (LAGGPGAG) or a fragment thereof, or having SEQ ID NO:34 (LGGGPG) or SEQ ID NO:35 (LAGGPG) or a fragment thereof, preferably wherein the peptide or fragment is selected from the group consisting of SEQ ID NO:36 (LGGGP), SEQ ID NO:37 (LAGGP), SEQ ID NO:38 (GGGP), and SEQ ID NO:39 (GAGP), or of a retro-inverso variants of SEQ ID NO:32 (LGGGPGAG) or a fragment thereof, or SEQ ID NO:33 (LAGGPGAG) or a fragment thereof, or of SEQ ID NO:34 (LGGGPG) or SEQ ID NO:35 (LAGGPG) or a fragment thereof, preferably wherein the peptide or fragment is selected from the group consisting of retro-inverso variants of SEQ ID NO:36 (LGGGP), SEQ ID NO:37 (LAGGP), SEQ ID NO:38 (GGGP) or SEQ ID NO:39 (GAGP), the peptide or fragment or variant having the ability to interact with elastin receptor type binding or modulate inflammatory activity of innate immune cells, for treating diabetes, diabetic complications, or for reducing inflammatory activity or for preparing a medicament for treating diabetes and diabetic complications or for reducing inflammatory activity, the use preferably further comprising the use of insulin or an interleukin-1 receptor antagonist or an antibody directed against an interleukin-1, preferably directed against interleukin-1-beta. In a preferred embodiment, the medicament is utilized for treating type-1 diabetes, optionally with nephropathy, neuropathy or retinopathy or for retarding the development of late type-2 diabetic complications or the medicament is utilized for treating an inflammatory condition.

Provided is a product containing at least one peptide SEQ ID NO:32 (LGGGPGAG) or a fragment thereof, or SEQ ID NO:33 (LAGGPGAG) or a fragment thereof, or having SEQ ID NO:34 (LGGGPG) or SEQ ID NO:35 (LAGGPG) or a fragment thereof, preferably wherein the peptide or fragment is selected from the group consisting of SEQ ID NO:36 (LGGGP), SEQ ID NO:37 (LAGGP), SEQ ID NO:38 (GGGP), and SEQ ID NO:39 (GAGP), the peptide or fragment having the ability to interact with elastin receptor type binding or modulate inflammatory activity of innate immune cells, or a product containing a retro-inverso variants of SEQ ID NO:32 (LGGGPGAG) or a fragment thereof, or SEQ ID NO:33 (LAGGPGAG) or a fragment thereof, or of SEQ ID NO:34 (LGGGPG) or SEQ ID NO:35 (LAGGPG) or a fragment thereof, preferably wherein the peptide or fragment is selected from the group consisting of retro-inverso variants of SEQ ID NO:36 (LGGGP), SEQ ID NO:37 (LAGGP), SEQ ID NO:38 (GGGP) or SEQ ID NO:39 (GAGP), together with at least one additional active agent effective to combat diabetes or diabetic complications as a combined preparation for simultaneous, separate or sequential use in the treatment diabetes and/or diabetic complications. In a further embodiment, the product is provided with at least one additional active agent effective to treat microvascular disease. Such a product as provided herein may be used for treating nephropathy or for preparing a medicament for treating nephropathy, or for treating neuropathy or for preparing a medicament for treating neuropathy, or for treating retinopathy or for preparing a medicament for treating retinopathy. Such a product may additionally be used or provided with an agent for glycemic control, preferably insulin or an antidiabetic agent functionally equivalent to insulin, preferably wherein the antidiabetic agent comprises regular insulin or an insulin analogue such as insulin lispro, insulin glulisine, insulin aspart, insulin degludec, insulin glargine, or wherein the antidiabetic agent comprises a sulfonylurea or a meglitinide or metformin.

Provided is use of a peptide, such as v14 peptide (SEQ ID NO:131) or derivatives thereof for the production of a medicament, or use of that peptide for preventing or treating disease in a human, the peptide capable of blocking, inhibiting or preventing of binding of fragments of C-peptide and of elastin peptide to an elastin receptor. In a preferred embodiment, provided is such a peptide or use thereof for preventing or treating disease in a human, the peptide capable of blocking, inhibiting or preventing of binding of fragments of C-peptide to an elastin receptor.

Provided is a method for preventing or treating disease comprising providing a human with a peptide capable of blocking, inhibiting or preventing of binding of fragments of C-peptide (preferably fragments comprising a SEQ ID NO:40 (GGGPG) sequence) and of elastin peptide (preferably fragments comprising a SEQ ID NO:41 (VGVAPG) sequence) to an elastin receptor, preferably wherein the peptide fragments comprise a binding site allowing binding to an elastin receptor, preferably wherein the binding site comprises an amino acid sequence motif GxxP, allowing a type VIII beta-turn, preferably wherein the site comprises an amino acid sequence motif GxxPG.

Synthetic peptides with amino acid sequence motif GxxP, allowing a type VIII beta-turn, preferably wherein the site comprises an amino acid sequence motif GxxPG, as provided herein are useful in the treatment of inflammatory conditions, such as acute kidney injury, also in acute systemic inflammatory conditions such as, for example, sepsis or systemic inflammatory response syndrome (SIRS), leading to vascular damage and often aggravated by (multiple organ) organ failure, or inflammatory conditions with diabetes, when given with an anti-diabetic composition such as insulin. In a further embodiment of the disclosure, peptides with amino acid sequence motif GxxP, allowing a type VIII beta-turn, preferably wherein the site comprises an amino acid sequence motif GxxPG, as provided herein are encapsulated in an acid resistant capsule. Such (pharmaceutical) capsules are widely used in the pharmaceutical field as oral dosage forms for administration to humans and animals. Filled with a peptide according to the disclosure, such a capsule is useful for the enteral administration of a synthetic peptide provided with at least one, preferably two or three pentapeptide motifs GxxPG or xGxPG (G being glycine, P being proline, and x any amino acid), preferably wherein at least one amino acid at one position x is selected from the group of glycine, alanine, leucine, valine or isoleucine, the peptide also provided with at least one glutamine. Such administration would alleviate or treat diseases such as Crohn's disease in which gut endothelial cells need regeneration. Also, such administration would be useful in treating gastro-intestinal damage obtained after excess radiation. The peptides with amino acid sequence motif GxxP, allowing a type VIII beta-turn, preferably wherein the site comprises an amino acid sequence motif GxxPG, as provided herein may also be advantageously combined with other therapeutic immunomodulators, such as with immunomodulatory peptides, such as peptides with SEQ ID NO:1 (LQGV), AQG, or SEQ ID NO:2 (AQGV), or with other immunomodulators, such as with immunomodulatory antibodies or proteins directed against cytokines as TNF-alpha, IL-1 or IL-6.

Provided herein is a new target for control of metabolic syndrome and for the development of immune-modulatory peptides directed against metabolic syndrome and against microvascular complications in diabetes: C-peptide's interaction with the elastin receptor. This specification provides a substantial jump in thinking about the cause of metabolic syndrome and of microvascular complications in diabetes. The disclosure describes the presence of a canonical elastin receptor binding motif, GxxP or xGxxPG, in human C-peptide, a fact that has been overlooked by the medical community at large. What is more, the motif is located in a hydrophobic mid-portion of C-peptide that was already in 1997 identified as central to its biological activity, but rejected as possible receptor binding site. This disclosure shows that rejection to be invalid. The disclosure puts elastin receptor activation by C-peptide forward as cause of insulin resistance, hypertension and chronic-low over-repair in metabolic syndrome and ties this syndrome together with other conditions of insulin resistance, such as COPD due to smoking and exposure to fine particular matter, where elastin-derived peptides may activate the elastin receptor to cause insulin resistance and over-repair.

Certain embodiments of the disclosure provided herein provide methods for antagonist peptide development, treatment and/or prevention of metabolic syndrome. In certain embodiments, antagonist peptide development, treatment or prevention may be directed to one or more of insulin resistance, atherosclerosis, cardiovascular disease, and/or micro- and macrovascular pathologies associated with diabetes mellitus. All amino acid sequences herein are depicted in the one-letter-code. C-peptide is found with all mammals that produce insulin, as it is co-produced and co-excreted with insulin by-cells of the pancreas. Common human C-peptide's amino acid sequence is SEQ ID NO:1 (EAEDLQVGQVELGGGPGAGSLQPLALEGSLQ). It is herein disclosed that C-peptides from a wide variety of species bear elastin receptor binding motifs within their hydrophobic mid-portion (in humans SEQ ID NO:8 (GGGPGAG)).

A receptor for C-peptide is herein identified as the elastin binding protein that can be found in the elastin receptor complex as the alternatively spliced galactosidase derived from beta-galactosidase, encoded by the GLB1 gene (Ubiprot identifier P16278). The isoform 1 of the gene product relates to the beta galactosidase (beta-Gal) whereas the isoform 2 relates to the alternatively spliced galactosidase (S-Gal).

http://www.piphuman.eu/site/home.html

Beta-Gal (isoform 1) cleaves beta-linked terminal galactosyl residues from gangliosides, glycoproteins, and glycosaminoglycans, and is located mainly in the lysosomes.

Isoform 2 (S-Gal) has little or no beta-galactosidase catalytic activity, but plays functional roles in the formation of extracellular elastic fibers (elastogenesis) and in the development of connective tissue. S-Gal is considered identical to the elastin-binding protein (EBP), a major component of the non-integrin cell surface receptor expressed on fibroblasts, smooth muscle cells, chondroblasts, leukocytes, and certain cancer cell types. In elastin producing cells, EBP associates with tropoelastin intracellularly and functions as a recycling molecular chaperone that facilitates the secretions of tropoelastin and its assembly into elastic fibers.

In certain embodiments, the antagonist peptide may bind to or interact with either C-peptide or with the elastin receptor. Further, the antagonist may bind to the elastin receptor-binding motif in C-peptide or to the site in the elastin receptor that binds to the elastin receptor-binding motif. Alternatively, the antagonist may bind to a site proximal or distal to the elastin receptor-binding motif in C-peptide or to the site in the elastin receptor that binds to the elastin receptor-binding motif but allows action as an antagonist of the C-peptide/elastin receptor interaction. In this way, the antagonist may affect the interaction between C-peptide and the elastin receptor while not interfering with the interaction between the elastin and other binding partners.

Also provided are methods for treating a human suffering from or considered to be suffering from metabolic syndrome or a related disorder. Such methods typically include synthesizing or isolating an antagonist of the interaction or binding of C-peptide with an elastin receptor and providing the human with the antagonist. The antagonist may be mixed with a pharmacologically acceptable excipient and the resulting mixture may be labeled as suitable for treating metabolic syndrome or a related disorder, or the prevention of metabolic syndrome.

Disclosed are methods for treating a human considered to be at risk of suffering from metabolic syndrome or a related disorder. Such methods typically include synthesizing or isolating an antagonist of the interaction or binding of C-peptide with an elastin receptor and providing the human with the antagonist. The antagonist may be mixed with a pharmacologically acceptable excipient and the resulting mixture may be labeled as suitable for a human considered to be at risk of suffering from metabolic syndrome or a related disorder.

Disclosed are methods for treating a human suffering from or considered to be suffering from type-2 diabetes. Such methods typically include synthesizing or isolating an antagonist of the interaction or binding of C-peptide with an elastin receptor and providing the human with the antagonist. The antagonist may be mixed with a pharmacologically acceptable excipient and the resulting mixture may be labeled as suitable for treating type-2 diabetes, or the prevention of type-2 diabetes.

Disclosed are methods for treating a human considered to be at risk of suffering from type-2 diabetes. Such methods typically include synthesizing or isolating an antagonist of the interaction or binding of C-peptide with an elastin receptor and providing the human with the antagonist. The antagonist may be mixed with a pharmacologically acceptable excipient and the resulting mixture may be labeled as suitable for the treatment or the prevention of type-2 diabetes.

By way of non-limiting theory as to function, damage to and destruction of the beta-cells is not only causal in type-1 diabetes but is also seen in the development of diabetes types 1.5 and 2, and metabolic syndrome as a whole as well, and the phenomena seen with insulin resistance are secondary or parallel to initial events in the pancreatic beta-cells. The damage to and destruction of the beta-cells is primarily caused by an overproduction of C-peptide that is secreted by these cells, deposited in its periphery, and leading to low-grade and initially heterogenic chronic inflammation of beta-cells and islets of Langerhans by C-peptides interaction with cells bearing the elastin receptor, an interaction mediated in human C-peptides by binding of the receptor to the hydrophobic mid-portion SEQ ID NO:32 (LGGGPGAG). Before, during or after these initial events or in conjunction therewith and when overproduction of C-peptide is maintained, low-grade inflammation is extended to peripheral tissues where C-peptide is deposited as well and again cells bearing the elastin receptor are stimulated. Embodiments herein provide antagonists of the C-peptide/EBP interaction and methods of treating a human so as to antagonize the C-peptide/EBP interaction.

In a further embodiment, provided are peptides derived from the human C-peptide (UIniprot identifier >sp|P01308|57-87) or functional mammalian equivalents thereof consisting of an octapeptide, hexapeptide, heptapeptide, pentapeptide or tetrapeptide comprising a GxxP or xGxP motif wherein G is glycine, P is proline, and x is any amino acid, and retro-inverso variants of the octapeptide, hexapeptide, heptapeptide, pentapeptide or tetrapeptide comprising the xGxP or GxxP motif. Note that no stereoisomers of glycine exist, herein L-glycine and D-glycine both stand for glycine. By way of non-limiting example, the peptides may be synthesized by a solid-phase method with an automated peptide synthesizer (such as model 990; Beckman Instrument, Fullerton, CA). The peptides may be purified by reverse phase high-performance liquid chromatography (such as Capcell Pak C-18, Shiseido, Tokyo, Japan). The sequence of the peptide may be confirmed with a mass spectrometer (such as Voyager, Linear-DE/K, Preseptive Biosystems, TX).

An exemplary octapeptide as provided herein comprising the xGxP or GxxP motif derived from the human C-peptide is the octapeptide SEQ ID NO:32 (LGGGPGAG) that is selected from the human C-peptide sequence as a whole and very well suited for human use considering its 100% homology over the stretch of 8 amino acids in C-peptide from which it is derived. Also provided is the retro-inverso variant, the all-D-amino acid peptide GAGPGGGL, and peptides or peptidometics of at most 30, preferably of at most 25, preferably of at most 20, preferably of at most 12, preferably of at most 9 amino acids, comprising the all-D-amino acid peptide GAGPGGGL. Note: no stereoisomers of glycine exist, here (and in retro-inverso peptides bearing, for example, retro-inverso GxxP or xGxP motifs) G is not, whereas other amino acids, such as L, P and A are, instrumental to the all-D-amino acid character of the retro-inverso peptide herein provided.

An exemplary hexapeptide as provided herein comprising the xGxP or GxxP motif derived from the human C-peptide is the hexapeptide SEQ ID NO:8 (GGGPGAG) that is selected from the human C-peptide sequence as a whole and very well suited for human use considering its 100% homology over the stretch of 7 amino acids in C-peptide from which it is derived. Also provided is the retro-inverso variant, the all-D-amino acid peptide GAGPGGG, and peptides or peptidometics of at most 30, preferably of at most 25, preferably of at most 20, preferably of at most 12, preferably of at most 8 amino acids, comprising the all-D-amino acid peptide GAGPGGG.

Another exemplary hexapeptide as provided herein comprising the xGxP or GxxP motif derived from the human C-peptide is the heptapeptide SEQ ID NO:47 (LGGGPGA) that is selected from the human C-peptide sequence as a whole and very well suited for human use considering its 100% homology over the stretch of 7 amino acids in C-peptide from which it is derived. Also provided is the retro-inverso variant, the all-D-amino acid peptide GAGPGGG, and peptides or peptidometics of at most 30, preferably of at most 25, preferably of at most 20, preferably of at most 12, preferably of at most 9, preferably of at most 7 amino acids, comprising the all-D-amino acid peptide AGPGGGL.

A most exemplary heptapeptide as provided herein comprising the xGxP or GxxP motif derived from the human C-peptide is the hexapeptide SEQ ID NO:48 (GGPGAG) that is selected from the human C-peptide sequence as a whole and very well suited for human use considering its 100% homology over the stretch of 6 amino acids in C-peptide from which it is derived. Also provided is the retro-inverso variant, the all-D-amino acid peptide GAGPGG, and peptides or peptidometics of at most 30, preferably of at most 25, preferably of at most 20, preferably of at most 12, preferably of at most 9, preferably of at most 6 amino acids, comprising the all-D-amino acid peptide GAGPGG.

Another exemplary heptapeptide as provided herein comprising the xGxP or GxxP motif derived from the human C-peptide is the heptapeptide SEQ ID NO:34 (LGGGPG) that is selected from the human C-peptide sequence as a whole and very well suited for human use considering its 100% homology over the stretch of 6 amino acids in C-peptide from which it is derived. Also provided is the retro-inverso variant, the all-D-amino acid peptide GPGGGL, and peptides or peptidometics of at most 30, preferably of at most 25, preferably of at most 20, preferably of at most 12, preferably of at most 9, preferably of at most 6 amino acids, comprising the all-D-amino acid peptide GPGGGL.

Another exemplary heptapeptide as provided herein comprising the xGxP or GxxP motif derived from the human C-peptide is the hexapeptide SEQ ID NO:49 (GGGPGA) that is selected from the human C-peptide sequence as a whole and very well suited for human use considering its 100% homology over the stretch of 6 amino acids in C-peptide from which it is derived. Also provided is the retro-inverso variant, the all-D-amino acid peptide AGPGGG, and peptides or peptidometics of at most 30, preferably of at most 25, preferably of at most 20, preferably of at most 12, preferably of at most 9, preferably of at most 6 amino acids, comprising the all-D-amino acid peptide AGPGGG.

A most exemplary pentapeptide as provided herein comprising the xGxP or GxxP motif derived from the human C-peptide is the heptapeptide SEQ ID NO:40 (GGGPG) that is selected from the human C-peptide sequence as a whole and very well suited for human use considering its 100% homology over the stretch of 6 amino acids in C-peptide from which it is derived. Also provided is the retro-inverso variant, the all-D-amino acid peptide GPGGG, and peptides or peptidometics of at most 30, preferably of at most 25, preferably of at most 20, preferably of at most 12, preferably of at most 9, preferably of at most 5 amino acids, comprising the all-D-amino acid peptide GPGGG.

Another exemplary pentapeptide is peptide SEQ ID NO:46 (GAGPG), and peptides or peptidometics of at most 30, preferably of at most 25, preferably of at most 20, preferably of at most 12, preferably of at most 9, preferably of at most 5 amino acids, comprising the peptide SEQ ID NO:46 (GAGPG).

Another exemplary pentapeptide is a retro-inverso variant, the all-D-amino acid peptide GPGAG, and peptides or peptidometics of at most 30, preferably of at most 25, preferably of at most 20, preferably of at most 12, preferably of at most 9, preferably of at most 5 amino acids, comprising the all-D-amino acid peptide GPGAG.

A most exemplary tetrapeptide as provided herein comprising the xGxP or GxxP motif derived from the human C-peptide is the tetrapeptide SEQ ID NO:38 (GGGP) that is selected from the human C-peptide sequence as a whole and very well suited for human use considering its 100% homology over the stretch of 6 amino acids in C-peptide from which it is derived. Also provided is the retro-inverso variant, the all-D-amino acid peptide PGGG, and peptides or peptidometics of at most 30, preferably of at most 25, preferably of at most 20, preferably of at most 12, preferably of at most 9, preferably of at most 4 amino acids, comprising the all-D-amino acid peptide PGGG.

Another tetrapeptide as provided herein comprising the GxxP motif is the tetrapeptide SEQ ID NO:39 (GAGP). Also provided is the retro-inverso variant, the all-D-amino acid peptide PGAG, and peptides or peptidometics of at most 30, preferably of at most 25, preferably of at most 20, preferably of at most 12, preferably of at most 9, preferably of at most 4 amino acids, comprising the all-D-amino acid peptide PGAG.

In certain embodiments is provided the use of a peptide derived from the human C-peptide (UIniprot identifier >sp|P01308|57-87) or from functional mammalian equivalents thereof consisting of an octapeptide, hexapeptide, heptapeptide, pentapeptide or tetrapeptide comprising a GxxP or xGxP motif wherein G is glycine, P is proline, and x is any amino acid, and retro-inverso variants of the octapeptide, hexapeptide, heptapeptide, pentapeptide or tetrapeptide comprising the xGxP or GxxP motif for treating metabolic syndrome, preferably of diabetes mellitus, preferably of type-1 diabetes or of nephropathies, neuropathies or microvascular disease associated with type-1 diabetes.

As such, provided herein are methods for treating a human for type-1 diabetes. Such methods typically include administering to the human a peptide derived from the human C-peptide (UIniprot identifier >sp|P01308|57-87) or from functional mammalian equivalent thereof consisting of an octapeptide, hexapeptide, heptapeptide, pentapeptide or tetrapeptide comprising a GxxP or xGxP motif wherein G is glycine, P is proline, and x is any amino acid, and retro-inverso variants of the octapeptide, hexapeptide, heptapeptide, pentapeptide or tetrapeptide comprising the xGxP or GxxP motif. In certain embodiments, the methods of treatment may be for one or more pathology associated with type-1 diabetes including, but not limited to, nephropathies, neuropathies or microvascular disease.

In certain embodiments, provided are peptides derived from the human C-peptide (UIniprot identifier >sp|P01308|57-87) or from functional mammalian equivalent thereof consisting of an octapeptide, hexapeptide, heptapeptide, pentapeptide or tetrapeptide comprising a GxxP or xGxP motif wherein G is glycine, P is proline, and x is any amino acid, and retro-inverso variants of the octapeptide, hexapeptide, heptapeptide, pentapeptide or tetrapeptide comprising the xGxP or GxxP motif for treating metabolic syndrome, preferably of diabetes mellitus, preferably of type-1 diabetes or of nephropathies, neuropathies or microvascular disease associated with type-1 diabetes.

Also provided is the use of a peptide derived from the human C-peptide (UIniprot identifier >sp|P01308|57-87) or from functional mammalian equivalent thereof consisting of an octapeptide, hexapeptide, heptapeptide, pentapeptide or tetrapeptide comprising a GxxP or xGxP motif wherein G is glycine, P is proline, and x is any amino acid, and retro-inverso variants of the octapeptide, hexapeptide, heptapeptide, pentapeptide or tetrapeptide comprising the xGxP or GxxP motif for producing a medicament for treating metabolic syndrome, preferably of diabetes mellitus, preferably of type-1 diabetes or of nephropathies, neuropathies or microvascular disease associated with type-1 diabetes.

As such, provided herein are methods for producing a medicament for treating of a human for type-1 diabetes. Such methods typically include synthesizing or isolating a peptide derived from the human C-peptide (UIniprot identifier >sp|P01308|57-87) or from functional mammalian equivalent thereof consisting of an octapeptide, hexapeptide, heptapeptide, pentapeptide or tetrapeptide comprising a GxxP or xGxP motif wherein G is glycine, P is proline, and x is any amino acid, and retro-inverso variants of the octapeptide, hexapeptide, heptapeptide, pentapeptide or tetrapeptide comprising the xGxP or GxxP motif.

In beta-cells, insulin is produced in conjunction with C-peptide from the same precursor molecule, pre-proinsulin. Both insulin and C-peptide are excreted in equal amounts into the blood, whereby insulin can act on peripheral tissues for a short time only, having a half-life of approximately 5 minutes. C-peptide, however, has a reported half-life of 30 minutes, and circulates much longer in the blood. Although C-peptide's function has long not been understood, many activities have currently been ascribed to it, including pro-inflammatory activities. The source of theses pro-inflammatory activities has not been explained. As provided herein, structural analysis of C-peptide reveals the presence of an elastin receptor-binding motif, which, without being bound to a particular theory, is the cause of the pro-inflammatory effect.

Common human C-peptide's amino acid sequence is: SEQ ID NO:1 (EAEDLQVGQVELGGGPGAGSLQPLALEGSLQ). The mid-portion, SEQ ID NO:42 (ELGGGPGAGS), bears an additional, hitherto unnoticed, characteristic PG-domain that explains the so-called pro-inflammatory character of C-peptide, and, in particular, explains the low-grade, and initially heterogenic, chronic inflammation (herein identified as blood vessel over-repair) that is seen with diabetes type-2 and metabolic syndrome, and more in particular, explains the onset, the etiology, of diabetes type-2 as a whole. It is herein recognized that SEQ ID NO:42 (ELGGGPGAGS) bears a canonical xGxP, GxxP, GxxPG and xGxPG (x being any amino acid, preferably a hydrophobic amino acid) sequence found in peptides reactive with the elastin binding protein, EBP. The elastin binding protein (EBP), a spliced variant of lysosomal beta-galactosidase, is the primary receptor of elastin peptides that, for example, have been linked to emphysema, aneurism and cancer progression. The sequences recognized by EBP share the GxxP consensus pattern found in numerous matrix proteins, notably in elastin where the SEQ ID NO:41 (VGVAPG) motif is repeated. Herein, C-peptide is recognized for the first time as being a ligand of EBP or the elastin receptor. C-peptide thus has a same set of chemotaxic, matrix-metallo-proteinase (MMP) activating, proliferative and low-inflammatory or vascular repair activities that known elastin-peptides derived from extra-cellular matrix (ECM) proteins have. This receptor interaction has not publicly been recognized before, neither by persons skilled in the art of diabetes research or of metabolic disorder research, nor by those skilled in the art of ECM or elastin peptide research.

Having identified a binding site EBP in C-peptide, the vascular repair or low-inflammatory modulation by C-peptide is now explained. Pericytes, smooth muscle cells, fibroblasts, adipose tissue cells, pancreatic stellate cells, and other connective tissue cells, together with endothelial cells, and circulating innate immune cells such as leucocytes and monocytes, may respond to binding of EBP to GxxPG bearing proteins and peptides by interleukin-1-beta mediated proliferation and low-grade inflammatory activation. Analysis of the human proteome shows that proteins with multiple GxxPG motifs are highly related to the extracellular matrix (ECM). Matrix proteins with multiple GxxP, xGxP or GxxPG sites include fibrillin-1, -2, and -3, elastin, fibronectin, laminin, and several tenascins and collagens.

Secondly, circulating leucocytes and monocytes show strong chemotaxis to GxxP, xGxP, GxxPG or xGxPG bearing proteins and peptides, C-peptide will thus attract those cells to wherever C-peptide is present.

Thirdly, binding of EBP to GxxP, xGxP, xGxPG or GxxPG bearing proteins and peptides has been associated with shedding of EBP from cellular surfaces and increased presentation of the interleukin-I receptor having affinity for interleukin-1-beta, allowing for hampered endocytosis or for a continued interleukin-1-beta mediated proliferation and inflammatory activation wherever C-peptide deposits are present.

Fourthly, binding of EBP to GxxP or GxxPG bearing proteins and peptides has been associated with the activation of neuraminidase and the release of sialic acid from proteins that induces insulin resistance, in particular, of adipocytes and hepatocytes.

Fifthly, binding EBP to GxxP or GxxPG bearing proteins and peptides has been associated with shedding of EBP from cellular surfaces and decreased presentation of PPCA having proteolytic activity toward endothelin-1, whereby increased endothelin-1 levels due to decreased proteolytic activity of PPCA result in increased hypertension.

Embodiments relate also in part to the identification of a receptor type that binds C-peptides or C-peptide fragments, thus inducing C-peptide related bioactivity associated with various disorders, such as immune disorders such as metabolic syndrome and diabetes. Such a receptor type that binds or interacts with C-peptides or C-peptide fragments as disclosed herein, is a mammalian elastin-receptor known to bind elastin peptides including but not limited to the elastin receptor complex, including a 67-kDa elastin-binding protein (EBP) identified as an spliced variant of beta-galactosidase, and related homologues and isoforms thereof, that is ubiquitously found on innate immune cells, extra cellular matrix cells, fibroblasts, vascular smooth muscle cells and certain tumor cells. Also binding these motifs are pancreatic elastases, herein understood to also have elastin binding protein type binding activity. Elastin binding protein type, typically binds to canonical xGxP, GxxP, GxxPG and xGxPG (x being any amino acid, preferably an hydrophobic amino acid) motifs in extracellular matrix proteins, such as elastin, laminins, collagen type IV, and fibrillin-1, and such a motif are herein for the first time identified in C-peptides. Elastin binding protein/elastin peptide interaction can also be found with integrins and galectin, EBP, integrins and galectins and other receptors capable of binding to xGxP, GxxP, GxxPG and xGxPG motifs herein commonly called elastin binding protein type. The identification of an “elastin receptor” that interacts with C-peptide to promote a biological response modulating associated with metabolic and immune disorders in turn provides a valuable and essential component when practicing additional embodiments, including but not necessarily limited to methods, uses and identified peptides for treating various disorders.

Certain embodiments relate to methods of identifying modulators of an “elastin binding protein type,” a receptor type disclosed herein as interacting with C-peptides or C-peptide fragments to promote an anti-inflammatory effect associated with alleviation of nephropathies and endothelial dysfunction in type-1 diabetes. A modulator of particular interest is a peptide that acts as an agonist to the elastin binding protein type. Such an agonist may be useful in the treatment of type-1 diabetes or other disorders characterized by relative or absolute C-peptide deficiency such as late-phase type-2 diabetes. While not being bound by theory, such a peptide will show the ability to mediate a signal to an extra-cellular matrix cell or white blood cell (such as a fibroblast or monocyte cell) causing chemotaxic and proliferative effects, for example, causing leucocyte chemotaxis or smooth muscle cell or fibroblast proliferation. An agonist can, for example, be selected from GxxP, xGxPG, GxxPx, GxxPG, xGxxP, xGxxPx, xGxxPG motif bearing peptides, such as peptides SEQ ID NO:32 (LGGGPGAG), SEQ ID NO:8 (GGGPGAG), SEQ ID NO:49 (GGGPGA), SEQ ID NO:38 (GGGP), SEQ ID NO:40 (GGGPG), SEQ ID NO:46 (GAGPG), SEQ ID NO:50 (GGGPE), SEQ ID NO:51 (GAIPG), SEQ ID NO:52 (GGVPG), SEQ ID NO:53 (GVAPG), SEQ ID NO:54 (YTTGKLPYGYGPGG), SEQ ID NO:55 (YGARPGVGVGIP), SEQ ID NO:56 (PGFGAVPGA), SEQ ID NO:57 (GVYPG), SEQ ID NO:58 (GFGPG), SEQ ID NO:59 (GVLPG), SEQ ID NO:51 (GAIPG), SEQ ID NO:60 (PGAIPG), SEQ ID NO:61 (PGAVGP), SEQ ID NO:62 (VGAMPG), SEQ ID NO:63 (VGSLPG), SEQ ID NO:64 (VGMAPG), SEQ ID NO:65 (VPGVG), SEQ ID NO:66 (IPGVG), SEQ ID NO:63 (VGSLPG), SEQ ID NO:41 (VGVAPG), SEQ ID NO:67 (VGVPG), SEQ ID NO:68 (AGAIPG), SEQ ID NO:69 (VPGV), SEQ ID NO:70 (LGITPG), SEQ ID NO:71 (GDNP), SEQ ID NO:72 (GAIP), SEQ ID NO:73 (GKVP), SEQ ID NO:74 (GVQY), SEQ ID NO:75 (GVLP), SEQ ID NO:76 (GVGP), SEQ ID NO:77 (GFGP), SEQ ID NO:78 (GGIP), SEQ ID NO:79 (GVAP), SEQ ID NO:80 (GIGP), SEQ ID NO:39 (GAGP), SEQ ID NO:81 (GGIPP), SEQ ID NO:82 (GQFP), SEQ ID NO:83 (GLSP), SEQ ID NO:84 (GPQP), SEQ ID NO:85 (GGPQP), SEQ ID NO:86 (GPQPG), SEQ ID NO:87 (GGPQPG), SEQ ID NO:88 (GIPP), SEQ ID NO:89 (GIPPA), SEQ ID NO:90 (GGIPPA) or SEQ ID NO:91 (GGYPGASYPGAYPGQAPPGAYPGQAPPGAYPGAPGAYPGAPAPGVYPGPPSGPGAYPS) or SEQ ID NO:92 (GGYPGASYP) or SEQ ID NO:93 (GAYPGQAPP) or SEQ ID NO:94 (GAYPGQA) or SEQ ID NO:95 (GAYPGAP) or SEQ ID NO:96 (GAYPG) or SEQ ID NO:97 (APAPGVYPG) or SEQ ID NO:98 (GAYPS) or retro-inverso or otherwise functionally related peptides thereof. While not being bound by theory, such an agonist peptide will show the ability to mediate a signal to an extra-cellular matrix cell or white blood cell (such as a fibroblast or monocyte cell) stimulating chemotaxic and proliferative effects, for example, stimulating leucocyte chemotaxis or smooth muscle cell or fibroblast proliferation.

Another modulator of particular interest is a peptide that acts as an antagonist to the elastin binding protein type. Such an antagonist may be useful in the treatment or prevention of type-2 diabetes or other disorders characterized by relative or absolute C-peptide excess, such as atherosclerosis, rheumatoid arthritis, macrovascular disease and cardiovascular disease following the onset of metabolic syndrome. Useful antagonists may be selected from GxxP, xGxPG, GxxPx, GxxPG, xGxxP, xGxxPx, xGxxPG motif binding peptides or binding domains, such as (commonly called) V32- or V14-peptides and functional fragments and functional variants thereof as, for example, SEQ ID NO:99 (QTLPGSCGQVVGSPSAQDEASPLSEWRASYNSAGSNITDA), SEQ ID NO:100 (LPGSCGQVVGSPSAQDEASPLSEWRASYNSAG), SEQ ID NO:101 (VVGSPSAQDEASPLSEWRASY), SEQ ID NO:102 (VVGSPSAQDEASPLS), SEQ ID NO:103 (PSAQDEASPL), SEQ ID NO:104 (SPSAQDEASP), SEQ ID NO:105 (AQDEAS), SEQ ID NO:106 (PSAQ), SEQ ID NO:107 (SAQD), SEQ ID NO:108 (DEAS), SEQ ID NO:31 (QDEA), SEQ ID NO:109 (SPSA), SEQ ID NO:110 (VVGGTEAQRNSWPLQ), SEQ ID NO:111 (VVGGTEAQRNSWPSQ), SEQ ID NO:112 (TEAQRNSWP), SEQ ID NO:113 (AQRN), SEQ ID NO:114 (IVGGRRARPHAWPFM), SEQ ID NO:115 (VVGGEDAKPGQFPWQ), SEQ ID NO:116 (VVGGRVAQPNSWPWQ), SEQ ID NO:117 (RVAQPNSW), SEQ ID NO:118 (VVGGAEARRNSWPSQ), SEQ ID NO:119 (AEARRNSW), SEQ ID NO:120 (VVGGQEATPNTWPWQ), SEQ ID NO:121 (QEATPNTW), SEQ ID NO:122 (VVGGEEARPNSWPWQ), SEQ ID NO:123 (EEARPNSW), SEQ ID NO:124 (VVGGTEAGRNSWPSQ), SEQ ID NO:125 (TEAGRNSWP), SEQ ID NO:126 (EDYRPSQQDECSPRE), SEQ ID NO:127 (PSQQDECSP), SEQ ID NO:128 (QQDEC), QDE, or related peptides, such as retro-inverso variant peptides derived from above listed V32- or V14-peptides and functional fragments and functional variants thereof. Such retro-inverso peptides as disclosed herein preferably have core D-amino acid sequence AEDQ, such as retro-inverso V14 peptide variant all-D-LPSAEDQASPSGVV or all-D-PSAEDQASPS or all-D-WESLPSAEDQASPSGVVQGC. Functional fragments or variants are typically found in chemotaxis assay as provided herein testing the capacity of peptides to inhibit binding of human C-peptide through C-peptide's motif GxxPG to the human elastin receptor, such a peptide capable of reducing (reduces) chemotaxis activity of human C-peptide. While not wishing to being bound by theory, such an antagonist peptide will show the ability to modulate a signal to an extra-cellular matrix cell or white blood cell (such as a fibroblast or monocyte cell) inhibiting chemotaxic and proliferative effects, for example, inhibiting leucocyte or smooth muscle cell or fibroblast proliferation.

Other useful agonist or antagonist peptides may, for example, be found in silico employing the homology model of the elastin-binding site of human EBP. Blanchevoy et al recently built a homology model of this protein and showed docking of SEQ ID NO:41 (VGVAPG) in this model (Blanchevoye et al, INTERACTION BETWEEN THE ELASTIN PEPTIDE VGVAPG AND HUMAN ELASTIN BINDING PROTEIN, doi: 10.1074/jbc.M112.419929; the contents of which, such as the relevant atomic coordinates of the binding site, are herein included by reference).

The disclosure also relates to methods of treating one or more disorders related to C-peptide deficiency, such as type-1 diabetes, as disclosed herein, through administration to a human of a modulator (such as an elastin binding protein type agonist peptide) that activates an elastin binding protein type. Such an elastin binding protein type agonist peptide may be identified through the methods described herein and will be useful in treating disorders, including but not limited type-1 diabetes. In a particular embodiment, such an elastin binding protein agonist as provided herein may preferably be used together with an agonist of alpha-enolase, (for example, a peptide comprising SEQ ID NO:129 (LALEGSLQ) or the pentapeptide SEQ ID NO:6 (EGSLQ) or functional parts thereof) or an agonist of GPR 146. The present disclosure also provides use of a an agonist of elastin binding protein type for treating type-1 diabetes or a disorder comprising relative or absolute C-peptide deficiency, useful agonists can be selected from xGxP, GxxP, xGxPG, GxxPx, GxxPG, xGxxP, xGxxPx, xGxxPG motif bearing peptides, such as peptides SEQ ID NO:32 (LGGGPGAG), SEQ ID NO:8 (GGGPGAG), SEQ ID NO:49 (GGGPGA), SEQ ID NO:38 (GGGP), SEQ ID NO:40 (GGGPG), SEQ ID NO:46 (GAGPG), SEQ ID NO:50 (GGGPE), SEQ ID NO:51 (GAIPG), SEQ ID NO:52 (GGVPG), SEQ ID NO:53 (GVAPG), SEQ ID NO:54 (YTTGKLPYGYGPGG), SEQ ID NO:55 (YGARPGVGVGIP), SEQ ID NO:56 (PGFGAVPGA), SEQ ID NO:57 (GVYPG), SEQ ID NO:58 (GFGPG), SEQ ID NO:59 (GVLPG), SEQ ID NO:51 (GAIPG), SEQ ID NO:60 (PGAIPG), SEQ ID NO:61 (PGAVGP), SEQ ID NO:62 (VGAMPG), SEQ ID NO:63 (VGSLPG), SEQ ID NO:64 (VGMAPG), SEQ ID NO:65 (VPGVG), SEQ ID NO:66 (IPGVG), SEQ ID NO:63 (VGSLPG), SEQ ID NO:41 (VGVAPG), SEQ ID NO:67 (VGVPG), SEQ ID NO:68 (AGAIPG), SEQ ID NO:69 (VPGV), SEQ ID NO:70 (LGITPG), SEQ ID NO:71 (GDNP), SEQ ID NO:72 (GAIP), SEQ ID NO:73 (GKVP), SEQ ID NO:74 (GVQY), SEQ ID NO:75 (GVLP), SEQ ID NO:76 (GVGP), SEQ ID NO:77 (GFGP), SEQ ID NO:78 (GGIP), SEQ ID NO:79 (GVAP), SEQ ID NO:80 (GIGP), SEQ ID NO:39 (GAGP), SEQ ID NO:81 (GGIPP), SEQ ID NO:82 (GQFP), SEQ ID NO:83 (GLSP), SEQ ID NO:84 (GPQP), SEQ ID NO:85 (GGPQP), SEQ ID NO:86 (GPQPG), SEQ ID NO:87 (GGPQPG), SEQ ID NO:88 (GIPP), SEQ ID NO:81 (GGIPP), SEQ ID NO:89 (GIPPA), SEQ ID NO:90 (GGIPPA) or retro-inverso variants of peptides, such as peptides SEQ ID NO:32 (LGGGPGAG), SEQ ID NO:8 (GGGPGAG), SEQ ID NO:49 (GGGPGA), SEQ ID NO:38 (GGGP), SEQ ID NO:40 (GGGPG), SEQ ID NO:46 (GAGPG), SEQ ID NO:50 (GGGPE), SEQ ID NO:51 (GAIPG), SEQ ID NO:52 (GGVPG), SEQ ID NO:53 (GVAPG), SEQ ID NO:54 (YTTGKLPYGYGPGG), SEQ ID NO:55 (YGARPGVGVGIP), SEQ ID NO:56 (PGFGAVPGA), SEQ ID NO:57 (GVYPG), SEQ ID NO:58 (GFGPG), SEQ ID NO:59 (GVLPG), SEQ ID NO:51 (GAIPG), SEQ ID NO:60 (PGAIPG), SEQ ID NO:61 (PGAVGP), SEQ ID NO:62 (VGAMPG), SEQ ID NO:63 (VGSLPG), SEQ ID NO:64 (VGMAPG), SEQ ID NO:65 (VPGVG), SEQ ID NO:66 (IPGVG), SEQ ID NO:63 (VGSLPG), SEQ ID NO:41 (VGVAPG), SEQ ID NO:67 (VGVPG), SEQ ID NO:68 (AGAIPG), SEQ ID NO:69 (VPGV), SEQ ID NO:70 (LGITPG), SEQ ID NO:71 (GDNP), SEQ ID NO:72 (GAIP), SEQ ID NO:73 (GKVP), SEQ ID NO:74 (GVQY), SEQ ID NO:75 (GVLP), SEQ ID NO:76 (GVGP), SEQ ID NO:77 (GFGP), SEQ ID NO:78 (GGIP), SEQ ID NO:79 (GVAP), SEQ ID NO:80 (GIGP), SEQ ID NO:39 (GAGP), SEQ ID NO:81 (GGIPP), SEQ ID NO:82 (GQFP), SEQ ID NO:83 (GLSP), SEQ ID NO:84 (GPQP), SEQ ID NO:85 (GGPQP), SEQ ID NO:86 (GPQPG), SEQ ID NO:87 (GGPQPG), SEQ ID NO:88 (GIPP), SEQ ID NO:81 (GGIPP), SEQ ID NO:89 (GIPPA), SEQ ID NO:90 (GGIPPA) or SEQ ID NO:91 (GGYPGASYPGAYPGQAPPGAYPGQAPPGAYPGAPGAYPGAPAPGVYPGPPSGPGAYPS) or SEQ ID NO:92 (GGYPGASYP) or SEQ ID NO:93 (GAYPGQAPP) or SEQ ID NO:94 (GAYPGQA) or SEQ ID NO:95 (GAYPGAP) or SEQ ID NO:96 (GAYPG) or SEQ ID NO:97 (APAPGVYPG) or SEQ ID NO:98 (GAYPS) or related peptides (or retro-inverso variants thereof).

Also provided is use in human of a peptide having a sequence essentially being homologous to a fragment of mammalian insulin C-peptide, the peptide comprising the sequence SEQ ID NO:32 (LGGGPGAG) or a fragment thereof, or the sequence SEQ ID NO:33 (LAGGPGAG) or a fragment thereof, and having the ability to interact with elastin binding protein type binding or modulate inflammatory activity of innate immune cells, the disclosure preferably provides a peptide having, most preferably consisting of the sequence SEQ ID NO:34 (LGGGPG) or SEQ ID NO:35 (LAGGPG) or a fragment thereof, preferably the fragment is selected from SEQ ID NO:36 (LGGGP), SEQ ID NO:37 (LAGGP), SEQ ID NO:38 (GGGP), SEQ ID NO:130 (AGGP), and these may be combined, for example, with insulin of with interleukin-1 receptor antagonist.

Also provided is use in human of an isolated or synthetic peptide, essentially being homologous to a fragment of mammalian insulin C-peptide, the peptide comprising the sequence SEQ ID NO:32 (LGGGPGAG) or a fragment thereof, or the sequence SEQ ID NO:33 (LAGGPGAG) or a fragment thereof, and having the ability to interact with elastin binding protein type binding or modulate inflammatory activity of innate immune cells, the peptide preferably having, most preferably consisting of the sequence SEQ ID NO:34 (LGGGPG) or SEQ ID NO:35 (LAGGPG) or a fragment thereof, preferably wherein the fragment is selected from SEQ ID NO:36 (LGGGP), SEQ ID NO:37 (LAGGP), SEQ ID NO:38 (GGGP), SEQ ID NO:130 (AGGP) and these may be combined, for example, with insulin of with interleukin-1 receptor antagonist.

Also provided is use in human of a retro-inverso variant of a peptide or fragment relating to the hydrophobic mid-portion of C-peptide, examples are all-D-amino acid peptides GAGPGGGL, GAGPGGAL, AGPGGGL, GPGGGPA, GPGGAL, GPGGGL, GPGGG, GPGAG and these may be combined into a pharmaceutical composition, for example, with insulin of with interleukin-1 receptor antagonist. It is preferred that these retro-inverso variants, preferably for use treatment of microvascular complications in type 1 diabetes, are 4 to 8 amino acids in length. These variants are, for example, provided herein for treating diabetes and/or diabetic complications, or for reducing inflammatory activity, for example, in diabetes type 1.

Also provided is use in human of a pharmaceutical composition comprising at least one peptide or fragment selected from the group all-D-amino acid peptides GAGPGGGL, GAGPGGAL, AGPGGGL, GPGGGPA, GPGGAL, GPGGGL, GPGGG, GPGAG, together with at least one pharmaceutically acceptable carrier or excipient and the pharmaceutical may further comprise at least one additional active agent effective to combat diabetes or diabetic complications or to treat an inflammatory condition, for example, wherein the additional active agent is insulin or an interleukin-1 receptor antagonist or an antibody directed against an interleukin-1, preferably directed against interleukin-1-beta. Uses in a human of these compositions are provided for use in the treatment of diabetes and diabetic complications or for reducing inflammatory activity. Other uses are provided as well, such as wherein the medicament is used for treating type-1 diabetes, optionally with nephropathy, neuropathy or retinopathy or for retarding the development of late type-2 diabetic complications, or wherein the medicament is used for treating an inflammatory condition. The peptide may be used with at least one additional active agent effective to combat diabetes or diabetic complications as a combined preparation for simultaneous, separate or sequential use in the treatment diabetes and/or diabetic complications or with at least one additional active agent effective to rheumatoid arthritis or for preparing a medicament for treating rheumatoid arthritis for treating atherosclerosis or for preparing a medicament for treating atherosclerosis or for the treatment macrovascular disease or for preparing a medicament for the treatment macrovascular disease or for treating osteochondritis desiccants or for preparing a medicament for treating osteochondritis desiccants or for treating microvascular disease or for preparing a medicament for treating microvascular disease or for treating metabolic syndrome or for preparing a medicament for treating metabolic syndrome or for treating nephropathy or for preparing a medicament for treating nephropathy or for treating neuropathy or for preparing a medicament for treating neuropathy or for treating retinopathy or for preparing a medicament for treating retinopathy or for treating interleukine-1 mediated inflammation or for preparing a medicament for treating interleukine-1 mediated inflammation.

The disclosure also relates to methods of treating one or more disorders related to C-peptide excess, such as insulin resistance, hypertension, atherosclerosis and early phases of type-2 diabetes, as disclosed herein, through administration to a mammalian host (including but not limited to a human) of a modulator (such as an elastin binding protein type antagonist) that inhibits an elastin binding protein type, either directly, or indirectly by binding to C-peptide and blocking its actions. Such an elastin binding protein type antagonist is useful in treating disorders, including metabolic syndrome, type-2 diabetes, and related disorders. In a particular embodiment, such an elastin binding protein antagonist as provided herein may preferably be used together with an antagonist of alpha-enolase or of GPR 146. Useful elastin binding protein antagonists can be selected from peptides, such as SEQ ID NO:99 (QTLPGSCGQVVGSPSAQDEASPLSEWRASYNSAGSNITDA), SEQ ID NO:100 (LPGSCGQVVGSPSAQDEASPLSEWRASYNSAG), SEQ ID NO:101 (VVGSPSAQDEASPLSEWRASY), SEQ ID NO:102 (VVGSPSAQDEASPLS), SEQ ID NO:103 (PSAQDEASPL), SEQ ID NO:104 (SPSAQDEASP), SEQ ID NO:105 (AQDEAS), SEQ ID NO:106 (PSAQ), SEQ ID NO:107 (SAQD), SEQ ID NO:108 (DEAS), SEQ ID NO:31 (QDEA), SEQ ID NO:109 (SPSA), SEQ ID NO:110 (VVGGTEAQRNSWPLQ), SEQ ID NO:111 (VVGGTEAQRNSWPSQ), SEQ ID NO:112 (TEAQRNSWP), SEQ ID NO:113 (AQRN), SEQ ID NO:114 (IVGGRRARPHAWPFM), SEQ ID NO:115 (VVGGEDAKPGQFPWQ), SEQ ID NO:116 (VVGGRVAQPNSWPWQ), SEQ ID NO:117 (RVAQPNSW), SEQ ID NO:118 (VVGGAEARRNSWPSQ), SEQ ID NO:119 (AEARRNSW), SEQ ID NO:120 (VVGGQEATPNTWPWQ), SEQ ID NO:121 (QEATPNTW), SEQ ID NO:122 (VVGGEEARPNSWPWQ), SEQ ID NO:123 (EEARPNSW), SEQ ID NO:124 (VVGGTEAGRNSWPSQ), SEQ ID NO:125 (TEAGRNSWP), SEQ ID NO:126 (EDYRPSQQDECSPRE), SEQ ID NO:127 (PSQQDECSP), SEQ ID NO:128 (QQDEC), QDE, or retro-inverso or otherwise functionally related peptides thereof.

It is exemplary that the interleukin 1 receptor antagonist (IL-1Ra) is a recombinant protein (rIL-1Ra), preferably a recombinant human protein (rhIL-1Ra), preferably anakinra.

Provided is use in a human of an isolated fragment of a C-peptide as an agent that modulates binding or interaction of a C-peptide with an elastin binding protein and use of an isolated fragment of an elastin binding protein as an agent that modulates binding or interaction of a C-peptide with an elastin binding protein.

Useful antagonists to be included in a combination medicine for treatment of a human can be can be selected from GxxP, xGxPG, GxxPx, GxxPG, xGxxP, xGxxPx, xGxxPG motif binding peptides, such as SEQ ID NO:99 (QTLPGSCGQVVGSPSAQDEASPLSEWRASYNSAGSNITDA), SEQ ID NO:100 (LPGSCGQVVGSPSAQDEASPLSEWRASYNSAG), SEQ ID NO:101 (VVGSPSAQDEASPLSEWRASY), SEQ ID NO:102 (VVGSPSAQDEASPLS), SEQ ID NO:103 (PSAQDEASPL), SEQ ID NO:104 (SPSAQDEASP), SEQ ID NO:105 (AQDEAS), SEQ ID NO:106 (PSAQ), SEQ ID NO:107 (SAQD), SEQ ID NO:108 (DEAS), SEQ ID NO:31 (QDEA), SEQ ID NO:109 (SPSA), SEQ ID NO:110 (VVGGTEAQRNSWPLQ), SEQ ID NO:111 (VVGGTEAQRNSWPSQ), SEQ ID NO:112 (TEAQRNSWP), SEQ ID NO:113 (AQRN), SEQ ID NO:114 (IVGGRRARPHAWPFM), SEQ ID NO:115 (VVGGEDAKPGQFPWQ), SEQ ID NO:116 (VVGGRVAQPNSWPWQ), SEQ ID NO:117 (RVAQPNSW), SEQ ID NO:118 (VVGGAEARRNSWPSQ), SEQ ID NO:119 (AEARRNSW), SEQ ID NO:120 (VVGGQEATPNTWPWQ), SEQ ID NO:121 (QEATPNTW), SEQ ID NO:122 (VVGGEEARPNSWPWQ), SEQ ID NO:123 (EEARPNSW), SEQ ID NO:124 (VVGGTEAGRNSWPSQ), SEQ ID NO:125 (TEAGRNSWP), SEQ ID NO:126 (EDYRPSQQDECSPRE), SEQ ID NO:127 (PSQQDECSP), SEQ ID NO:128 (QQDEC), QDE, or related peptides and these may be combined into a pharmaceutical composition, for example, with insulin of with interleukin-1 receptor antagonist.

### BRIEF DESCRIPTION OF THE DRAWINGS

FIG. 1: Ido et al. (Science, 1997 Jul. 25; 277(5325):563-6) show human C-peptide's mid-portion SEQ ID NO:8 (GGGPGAG) to normalize glucose-induced vascular dysfunction. However, finding reverse (retro) and all-D-amino acid (enantio or inverso) C-peptides equipotent to native C-peptide, they conclude the activity of SEQ ID NO:8 (GGGPGAG) to be not mediated by a receptor, thereby teaching away from a receptor for C-peptide. This specification shows that the opposite is a case. All peptides with the mid-portion motif GxxP (SEQ ID NO:38 (GGGP) in human and rat C-peptide, SEQ ID NO:39 (GAGP) in reverse C-peptide and stereochemically equivalent PGAG in D-form C-peptide) normalize vascular dysfunction while none of the peptides without that motif do. Thus, the EBP-binding motif GxxP in SEQ ID NO:8 (GGGPGAG) is both necessary and sufficient to normalize vascular dysfunction. Hence, human C-peptide can be considered a ligand of the EBP that modulates vascular repair via the ERC. Efficacy is expressed as an average percent of the effect of 100 nM human C-peptide. Significantly different for 30 mM glucose: *P<0.05. [symbol] GxxP motifs in peptides bind to the elastin receptor when allowing for a close to a type VIII beta-turn confirmation, a condition considered always to be met by the motif xGxxPG. [symbol] All peptides having the GxxP motif (SEQ ID NO:38 (GGGP) or SEQ ID NO:39 (GAGP), or all-D PGAG which is stereomerically equivalent to all-L-SEQ ID NO:39 (GAGP)) show significant normalization of vascular dysfunction while none of the peptides without the motif show significant effects, illustrating that the elastin receptor binding motif GxxP is both necessary and sufficient to elicit the biological activity of C-peptide. Figure adapted from Ido Y, et al. Prevention of vascular and neural dysfunction in diabetic rats by C-peptide. Science 1997; 277: 563-66.

FIG. 2: Summarized description of pathological vascular effects of GxxP-peptide deficiency versus GxxP-excess

FIG. 3: Various GxxP hexa-peptides were docked in the peptide-binding site of the elastin binding protein (EBP) using Vina/Autodock and PyMOL (1, 2, 3). The binding conformation of each peptide was chosen from the top 20 best scoring poses. A homology model of EBP (4) was used as receptor in the docking procedure. Peptides tested were:

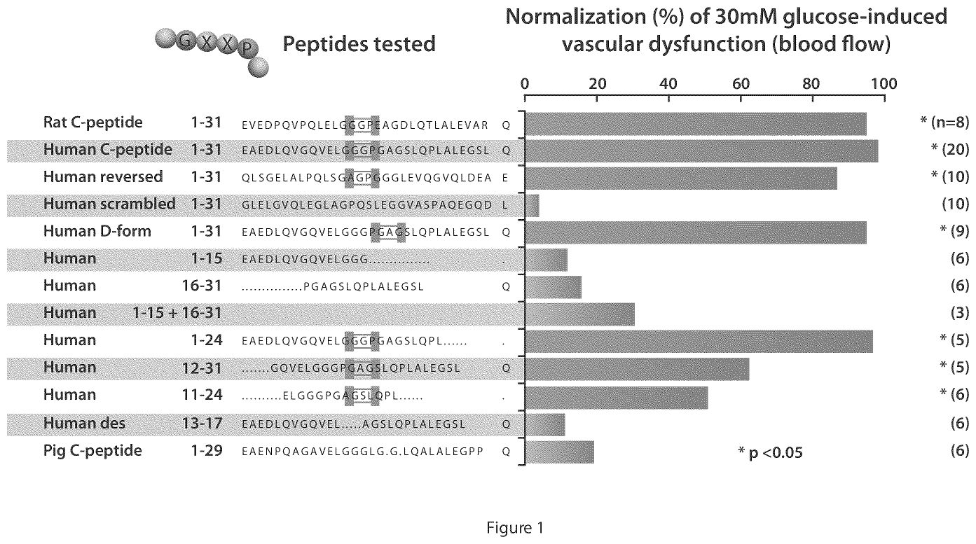

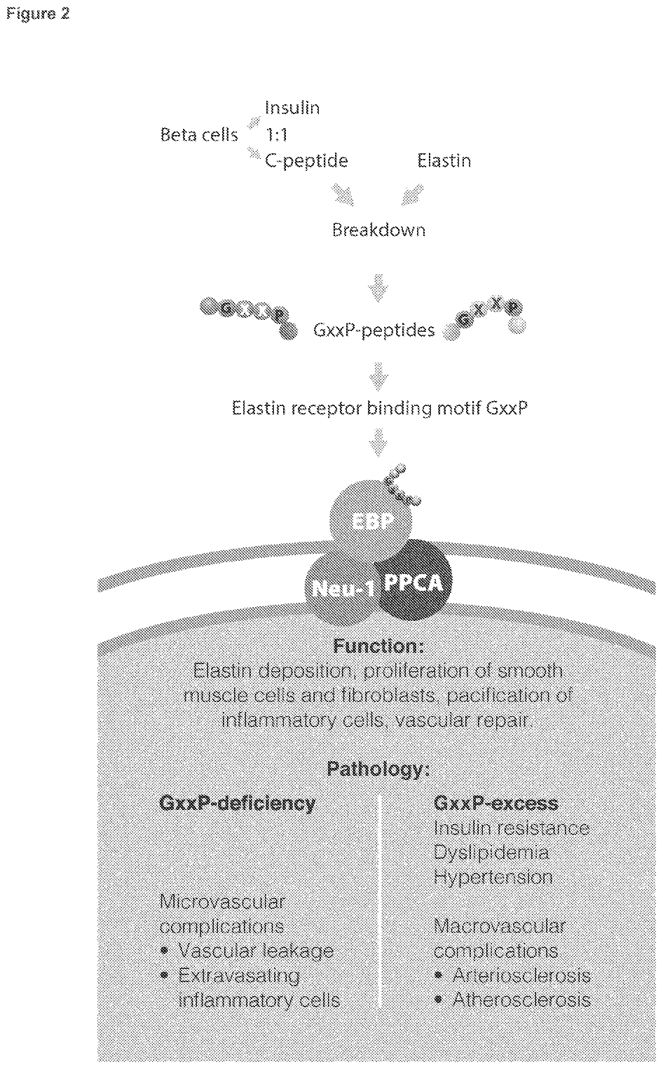

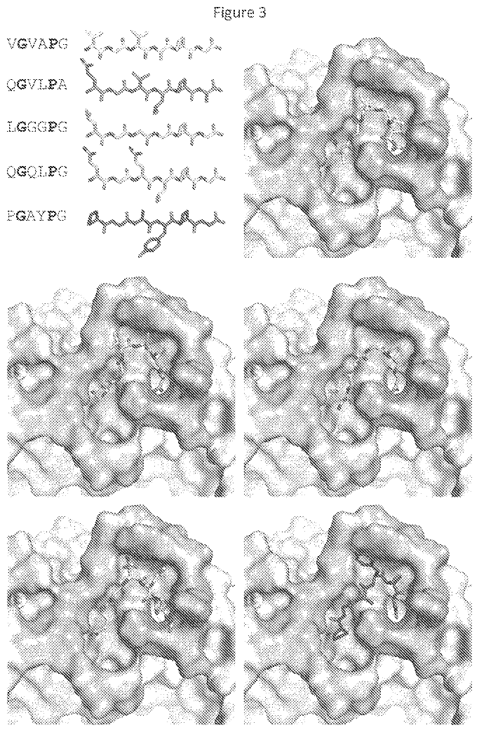

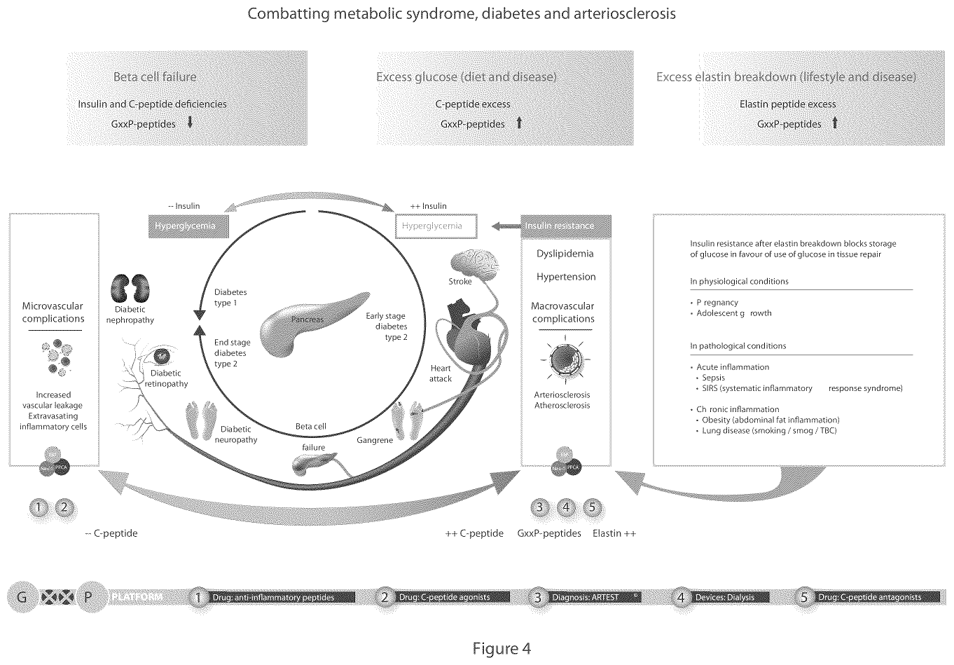

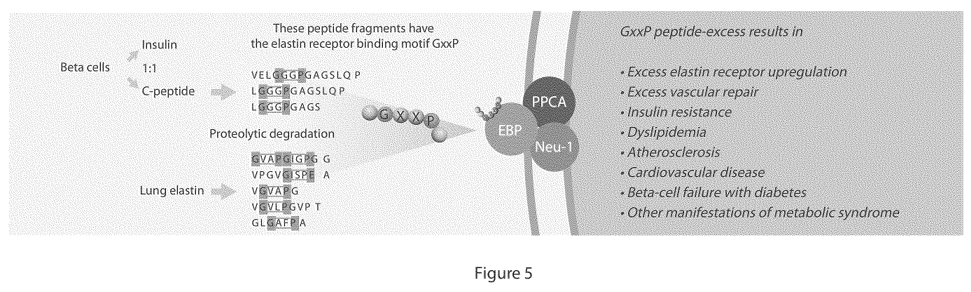

SEQ ID NO:41 (VGVAPG) (prototype GxxP-peptide ligand of EBP (4))

SEQ ID NO:34 (LGGGPG) (selected from human C-peptide (5))

SEQ ID NO:43 (QGQLPG) (immunomodulatory peptide provided herein)

SEQ ID NO:44 (PGAYPG) (selected from human Galectin-3 (6))

SEQ ID NO:45 (QGVLPA) (selected from loop 2 of human beta-hCG (7))

References [symbol] 1 Trott, O & Olson, A J (2010) AutoDock Vina: improving the speed and accuracy of docking with a new scoring function, efficient optimization and multithreading, J Comp Chem 31: 455-461.

[symbol]

2 Seeliger, D & de Groot, BL (2010) Ligand docking and binding site analysis with PyMOL and Autodock/Vina. J Comput-Aided Mol Des 24:417-422.

3 www.pymol.org

4 Blanchevoye, C et al. (2013) Interaction between the elastin peptide VGVAPG and human elastin binding protein, J Biol Chem 288:1317-28.

5 Ido, Y et al. (1997) Prevention of vascular and neural dysfunction in diabetic rats by C-peptide Science 277:563-6.

6 De Boer, R et al. (2011) Plasma Galectin-3 Is Associated with Near-Term Rehospitalization in Heart Failure: A Meta-Analysis Journal of Cardiac Failure Vol 17, Issue 8, S93

[symbol]

7 Khan, N A et al. (2010) Mitigation of septic shock in mice and rhesus monkeys by human chorionic gonadotrophin-related peptides, Clin Exp Immunol 160:465-478.

Similarly, All-D-amino acid peptide GPGAG fits in the model of EBP designed for docking prototype elastin peptide SEQ ID NO:41 (VGVAPG) as well. Also, L-amino acid peptides SEQ ID NO:40 (GGGPG) and SEQ ID NO:46 (GAGPG) fit the model as well. EBP-associated bioactivity is considered to depend on whether the GXXP-peptide can adapt to a type VIII beta-turn confirmation at the proline (P).

FIG. 4: Overview of pathophysiological pathways in metabolic syndrome, atherosclerosis and diabetes

FIG. 5: This new perspective sheds new light on diseases that are associated with atherosclerosis and insulin resistance (e.g., cardiovascular disease, stroke, peripheral arterial disease, dementia, chronic kidney disease and beta cell failure leading to diabetes). It not only ties together sugar and smoking but also various other co-existing risk factors that result from diet (excess intake of refined starches or of processed meat products with excess elastin), or lifestyle (exposure to smog or lack of exercise). The disclosure puts elastin receptor activation by C-peptide forward as cause of insulin resistance, hypertension and chronic-low inflammation or blood vessel over repair in metabolic syndrome and ties this syndrome together with other conditions of insulin resistance, such as COPD due to smoking and exposure to fine particular matter, where elastin-derived peptides may activate the elastin receptor to cause insulin resistance over-repair. FIG. 5 shows the binding motif GxxP in VELGGGPGAGSLQP (SEQ ID NO:145), LGGGPGAGSLQP (SEQ ID NO:146) and LGGGPGAGS (SEQ ID NO:147), GVAPGIGPGG (SEQ ID NO:144), VPGVGISPEA (SEQ ID NO:143), VGVAPG (SEQ ID NO:41), VGVLPGVPT (SEQ ID NO: 217), and GLGAFPA (SEQ ID NO: 218).

### DETAILED DESCRIPTION

Human C-peptide is found a ligand of the human elastin receptor.

Elastin receptor shall mean a chemical group or molecule on the cell
surface or in the cell interior that has an affinity for a peptide having an amino acid motif GxxP, wherein G represents the one-letter code for the amino acid glycine, P for the amino acid proline and x for any amino acid, the amino acid following P preferably allowing for a type VIII-beta turn, a condition that is met when P is C-terminally followed by a G, the elastin receptor typically represented in humans by the elastin binding protein known in the publicly accessible database UIniprot as GLB1—isoform 2 under identifier: P16278-2.

C-peptide
shall mean a peptide typically produced by beta-cells in the pancreas together with insulin, the C-peptide represented in humans by the peptide known in the publicly accessible database UIniprot as INS—isoform 1 under identifier: P01308-1, position 57-87.

Human C-peptide, connecting immature insulin chains A and B and secreted in a 1:1 ratio with mature insulin into the portal circulation, has traditionally been considered inert, despite ever increasing evidence of its biological activity. I show that in dietary excess, excess serum C-peptide leads to chronic-low grade inflammation, insulin resistance and hypertension and is causal to metabolic syndrome. I show C-peptide carrying a hitherto unrecognized xGxxPG motif specific for binding of elastin peptides to the elastin receptor, the receptor fulfilling various roles in tissue inflammation and tissue repair. Recent findings show this receptor to promote insulin resistance, dyslipidaemia, hypertension and atherogenesis, all characteristic of metabolic syndrome. This finding takes C-peptide into the limelight, tying in metabolic syndrome with other conditions of insulin resistance, such as COPD, when circulating elastin-derived peptides may combine with C-peptide to stimulate elastin receptor-mediated insulin resistance and inflammation.

### Insulin Resistance

Insulin resistance (IR) is central to metabolic syndrome^{1,2}. It occupies a crucial place in the aetiology of chronic inflammatory, lifestyle-, diet- or age-related, conditions as atherosclerotic cardiovascular disease and diabetes type 2. Hallmarks of metabolic syndrome are IR, hypertension, dyslipidemia, hyperinsulinemia, and impaired glucose tolerance. Uncertainties exist to the cause of IR. Simplified, the main view^{1,3} holds chronic-low grade inflammation to drive IR and subsequent hyperinsulinemia; a seemingly opposed view² holds increased hyperinsulinemia to drive IR and subsequent inflammation.

In humans in dietary excess, excess serum C-peptide causes chronic-low grade inflammation as well as IR and hypertension leading to metabolic syndrome, C-peptide being hitherto unrecognized as a ligand for the elastin receptor.

### The Elastin Receptor

The human elastin receptor⁴⁻⁶ is involved in chemotaxis of leukocytes and activation of matrix-metallo-proteinases, in endothelial cell migration and angiogenesis and in proliferation of fibroblasts and vascular smooth-muscle cells. The receptor is activated by (proteolytic) fragments of extracellular matrix in granulating tissue after tissue injury or inflammation, fulfilling handyman jobs toward tissue repair.

The receptor consists of an alternatively spliced variant of human beta-galactosidase. It binds to a hexapeptide x-Gly-x-x-Pro-Gly (xGxxPG) motif in (proteolytic fragments of) extracellular matrix proteins such as elastin and fibrillin-1⁴. The best-known representative of the motif is hexapeptide SEQ ID NO:41 (VGVAPG) found in (tropo) elastin, but many other biologically active peptides conforming to the signature sequence xGxxPG, generally called elastin peptides, have been reported as agonist^{4,5}. A minimally essential sequence for biological activity is GxxP, with the peptide at P adopting a type VIII beta-turn 5. V14 peptide (SEQ ID NO:131 (VVGSPSAQDEASPL)) corresponding to the binding site of the receptor, is used to antagonize elastin peptide binding⁶.

The elastin receptor forms a complex with neuraminidase (Neu-1) and protective protein-cathepsin A (PPCA) on the cell surface⁴. After binding to its ligand, the complex internalizes to endosomal compartments in the cell and triggers numerous cellular responses. In mice, elastin peptides potentiate atherosclerosis through Neu-1⁷ and regulate IR⁸ due to an interaction between Neu-1 and the insulin receptor. Moreover, in mice, PPCA is required for assembly of elastic fibers and inactivation of endothelin-1, impaired activation of endothelin-1 resulting in hypertension⁹.

### C-Peptide

Herein recognized, human C-peptide (₁SEQ ID NO:1 (EAEDLQVGQVELGGGPGAGSLQPLALEGSLQ)₃₁) contains the xGxxPG motif (underlined), surprisingly identifying it as a ligand for the elastin receptor. The far reaching implications of that find are discussed below. Classically, C-peptide connects the A- and B-chain of insulin in the pre-proinsulin produced in pancreatic beta-cells from the insulin gene and facilitates folding and binding of chains A and B. After processing, mature insulin and C-peptide are secreted into the portal circulation. Be it under dietary frugality or excess, insulin and C-peptide are produced and secreted in equimolar concentrations. However, C-peptide's plasma half-life of ˜30 min versus insulin's half-life of ˜4 min¹⁰ causes dietary excess to maintain persistently higher levels of circulating C-peptide than of insulin. The traditional view holds circulating C-peptide essentially inert and, because of its longer half-life, particularly useful as a surrogate marker of insulin release. However, accumulating evidence points at biological functions for C-peptide¹¹⁻¹⁴. Excess C-peptide in mice experimentally elicits inflammatory effects in vasculature and around glomeruli and C-peptide is found deposited in atherosclerotic lesions of patients¹⁵. Fasting serum C-peptide levels significantly relate to hazards of cardiovascular and overall death in non-diabetic adults¹⁶. These recent findings establish pathophysiological importance to C-peptide in its own right.

Pentapeptide₂₇ SEQ ID NO:6 (EGSLQ)₃₁

A first concerns is the pentapeptide ₂₇SEQ ID NO:6 (EGSLQ)₃₁, corresponding to the C-terminal five residues of human C-peptide that mimics several effects of the full-length peptide. The pentapeptide displaces cell membrane-bound C-peptide, increases intracellular Ca(2+) and stimulates MAP kinase signaling pathways and Na(+),K(+)-ATPase⁸. Of note, the glutamate at position 27 was shown essential to activation of alpha-enolase by C-peptide¹⁴, hinting that the C-terminal pentapeptide site may be involved in interaction of C-peptide with alpha-enolase.

Midportion ₁₃SEQ ID NO:8 (GGGPGAG)₁₉

A second site, and main focus of this disclosure, the mid-portion of human C-peptide 13 SEQ ID NO:8 (GGGPGAG)₁₉, was detected when structural features of C-peptide critical for mediating its effects on vascular dysfunction were investigated in a skin chamber granulation tissue model in rats¹⁴. ₁₃SEQ ID NO:8 (GGGPGAG)₁₉ was shown to be central to C-peptide's biological activity. However, as synthetic reverse sequence (retro) and all-D-amino acid (enantio) C-peptides were found equipotent to native C-peptide, it was concluded then 14 that the effects of this mid-portion must rely on non-chiral interactions, thereby teaching away from any possible stereospecific receptor binding to ₁₃SEQ ID NO:8 (GGGPGAG)₁₉. This teaching has since then dominated the literature on C-peptide. However, I here conclude that an xGxxPG elastin receptor binding motif is overlapping with C-peptide's bioactive mid-portion (₁SEQ ID NO:1 (EAEDLQVGQVELGGGPGAGSLQPLALEGSLQ)₃₁), distinctly associated with effects on vascular function in granulation tissue, identifying C-peptide as a biologically active ligand of the elastin receptor.

### C-Peptide Receptor

Until now, a distinct C-peptide receptor is unknown. A binding study 12 of C-peptide to human cell membranes indicates the existence of at least two C-peptide/receptor complexes, one with high affinity and low mobility and one with low affinity and high mobility and recent studies suggest alpha-enolase¹⁷, a cell surface receptor of plasminogen, or GPR146¹⁸, associated with dyslipidemia¹⁹, as possible receptor candidates for C-peptide. Biologically active sites in C-peptide. At least two biologically active sites have been identified in the C-peptide.

### Non-Chirality is Revoked

Surprisingly, studying reference 14 anew, the xGxxPG motif is also present in the biologically active retro C-peptide (₁SEQ ID NO:136 (QLSGELALPQLSGAGPGGGLEVQGVQLDEAE)₃₁). Also, the biologically active enantio C-peptide (D-₁EAEDLQVGQVELGGGPGAGSLQPLALEGSLQ₃₁) carries the motif, being hidden as the retro-enantio sequence D-GPGAGS; retro-enantio peptides being stereomerically nearly identical as their parent peptides, maintaining overall side-chain topology albeit for different N-terminal and C-terminal endings²⁰. These observations revoke the teaching¹⁴ of non-chirality and instead allow for stereospecific binding of these peptides to a receptor recognizing the motif: the elastin receptor.

### C-Peptide is a Species of the Genus of Elastin Peptides.

Moreover, additional examples of fragments of the human C-peptide are provided herein and¹⁴, all bearing a mid-portion hexapeptide ₁₂SEQ ID NO:34 (LGGGPG)₁₇, that all prevented vascular dysfunction whereas other human C-peptide fragments, wherein the hexapeptide mid-portion was disrupted, were found not active. Rat C-peptide, comprising a hexapeptide (₁₂SEQ ID NO:134 (LGGGPE)₁₇) GxxP motif (the P allowing a type VIII beta-turn required for biological activity 5) was found active as well, whereas pig C-peptide (mid-portion₁₂ SEQ ID NO:135 (LGGGLG)₁₇) not containing the essential P in the elastin binding motif, was found inactive. Of note, all 11 C-peptide (fragments) with the GxxP motif prevented vascular dysfunction, whereas all 5 without the motif did not, showing that even fragments of circulating C-peptide may contribute to elastin receptor activation, as long as the GxxP motif and the type VIII beta-turn is present. Human C-peptide and its xGxxPG containing fragments may thus be considered an unexpected species of the genus of a larger class of peptides: elastin peptides capable of elastin receptor activation, whereby excess C-peptide may be meddling with elastin receptor mediated tissue repair, modulating chronic-low grade inflammation, IR and hypertension. Insulin resistance extends beyond metabolic syndrome. Based on the above, I pose that in humans in dietary excess and prone to develop metabolic syndrome excess C-peptide binds to the elastin receptor, eliciting at least three effects, chronic-low grade inflammation (rather to be seen as excess vascular repair activity), insulin resistance and hypertension. The finding ties together conditions seen with metabolic syndrome with conditions possibly caused by circulating elastin degradation products, such as COPD, caused by smoking or by exposure to fine particulate matter (smog), or by physiological conditions, such as pregnancy and growth; allowing for a cumulative pathology when both C-peptide and elastin-derived peptides are increased. This provides a substantial jump in our understanding of the causes of metabolic syndrome and other lifestyle- or age-related conditions of IR. Elastin peptide/elastin receptor binding has been demonstrated for synthetic peptides such as SEQ ID NO:41 (VGVAPG) and inhibited by antagonist V14 peptide⁴⁻⁶. It is provided to do these tests with synthetic human C-peptide variants or fragments, provided with or without the sequence GxxP, to study binding, including classical elastin receptor antagonist V14 peptide to study inhibition of binding. Similarly, one can do the C-peptide tests in a skin chamber granulation tissue model of vascular function¹⁴, or test synthetic, inducibly or constitutively expressed C-peptide in established models of atherosclerosis, Neu-1 mediated IR or PPCA mediated hypertension⁷⁻⁹. For example, in a classical Boyden chamber experiment, a 100% increase of migration of CD4+ immune cells in 1% serum medium was demonstrated in vitro by C-peptide at 10 nM which CD4-migration was then inhibited, antagonized and diminished by >50% by V14-peptide at 1.3 microM, specifically demonstrating reduction of C-peptide-specific biological activity by elastin receptor antagonist V14 peptide.

### REFERENCES INCLUDED HEREIN ARE INCORPORATED BY REFERENCE

1 Shoelson S E, Lee J, Goldfine A B. J Clin Invest. 2006; 116:1793-1801.
2 Shanik M H, Xu Y, Skrha J, Dankner R, Zick Y, Roth J. Diabetes Care. 2008; Suppl 2:S262-8.
3 Bolton C E, Evans M, Ionescu A A, et al., COPD. 2007; June; 4(2):121-6.
4 Adair-Kirk T L, Senior R M. Int J Biochem Cell Biol. 2008; 40(5-7):1101-10.
5 Blanchevoye et al, idoi: 10.1074/jbc.M112.419929 jbc.M112.419929.
6 Robinet A, Fahem A, Cauchard J H, et al., J Cell Sci. 2005; Jan. 15; 118(Pt 2):343-56.
7 Gayral S, Garnotel R, Castaing-Berthou A, et al., Cardiovasc Res. 2013; Dec. 19. [Epub ahead of print]
8 Blaise S, Romier B, Kawecki C, et al., Diabetes. 2013; November; 62(11):3807-16.
9 Seyrantepe V, Hinek A, Peng J, et al., Circulation. 2008; Apr. 15; 117(15):1973-81.
10 Faber O K, Hagen C, Binder C, eta al., J Clin Invest 1978; 62: 197-203.
11 Hills C E, Brunskill N J. Clin Sci (Lond). 2009; April; 116(7):565-74.
12 Rigler, R., Pramanik, A. and Jonasson, P. Proc. Natl. Acad. Sci. U.S.A. 1999; 96, 13318-13323.
13 Johansson J, Ekberg K, Shafqat J, et al., Biochem Biophys Res Commun. 2002; August 2; 295(5):1035-40.
14 Y, Ido Vindigni A, Chang K, et al., Science. 1997; July 25; 277(5325):563-6.
15 Vasic D, Walcher D. Int J Inflam. 2012; 2012:932725.
16 Patel N, Taveira T H, Choudhary G, Whitlatch H, Wu W C. J Am Heart Assoc. 2012; December 1(6):e003152.
17 Ishii T, Fukano K, Shimada K, et al., J Biochem. 2012; July; 152(1):53-62.
18 Yosten G L, Kolar G R, Redlinger U, Samson W K. J Endocrinol. 2013; 218(2):B 1-8.
19 International Patent Application PCT/GB2006/003759 2005;
20 Muller S, Benkirane N, Guichard G, Van Regenmortel M H, Brown FExpert Opin Investig Drugs; 1998 September; 7(9):1429-38.

Again, when we eat too much, we provide the beta-cells of our pancreas with continued glucose signaling to produce insulin, to harbor the ever excess glucose derived from our food in peripheral liver, muscle, and fat cells. When eating in excess, we demand ever increasing insulin production from our beta-cells, and therewith demand ever increasing production of C-peptide from our beta-cells, C-peptide and insulin being produced and excreted in equal amounts. As insulin has a typical half-life of about 3-4 minutes, conditions of excess insulin may be easily coped with. However C-peptide has a much longer half-life, typically of >30 minutes, and depositions of excess C-peptide (and of partly or whole unprocessed pro-insulin) will be formed around the rim of the beta-cells and the islets of Langerhans, and also in the vascular wall of our blood-vessels. Pericytes, smooth muscle cells, fibroblasts, adipose tissue cells, pancreatic stellate cells, and others, together with endothelial cells, and possibly circulating leucocytes, respond to binding of EBP to GxxPG bearing C-peptide, thereby causing matrix-metallo-proteinase (MMP) induced hydrolysis accompanied by interleukin-1-beta mediated proliferation and subsequent low-grade inflammatory activation, in and around beta-cells in the islets of Langerhans. IL-1-beta thus drives tissue inflammation that impacts on both beta-cells functional mass and subsequently may also drive insulin sensitivity in type-2 diabetes. Binding of EBP to C-peptide's GxxPG sequences may further facilitate shedding of EBP from cellular surfaces and increased presentation of the interleukin-I receptor, allowing for a continued interleukin-1-beta mediated proliferation and inflammatory activation wherever C-peptide deposits are present, again driving insulin sensitivity in type-2 diabetes. In a patient thus developing diabetes type-2 or metabolic syndrome damage to and destruction of the beta-cells in the pancreas is following excess C-peptide production by those cells. Phenomena commonly seen as insulin resistance are then often secondary to initial events in the pancreatic beta-cells and arise out of interaction of fibroblasts, smooth muscle cells pericytes and leucocytes with vascular or peripheral C-peptide overload.

C-peptide exerts chemotactic and bioactive effects via interaction of its GXXPG and XGXPG motif with the elastin-binding protein. It is taught in this disclosure that C-peptide exerts chemotactic and bioactive influence on monocytes, pericytes, smooth muscle cells, fibroblasts, and other cells via interaction with the elastin-binding protein (EBP) (Privitera et al., J. Biol. Chem. 1998; 273:6319-6326). This receptor recognizes Gly-X—X-Pro-Gly (XGXXPG) or X-Gly-X-Pro-Gly (XGXPG) motifs found in C-peptides, wherein X can be any amino acid, and preferably a hydrophobic amino acid. The identity of this receptor protein, commonly called the elastin binding protein (EBP), has been established as an enzymatically inactive, alternatively spliced variant of beta-galactosidase. EBP forms a complex with protective protein/cathepsin A (PPCA) and lysosomal sialidase (neuraminidase-1, Neu-1). As C-peptide is released in equimolar concentrations together with insulin, but has a much longer half-life, increased insulin excretion as result of increased food-intake will result in even higher C-peptide levels. This evokes an oversupply of C-peptide and deposits of the C-peptide are observed in the periphery of beta-cells and even in the (micro)vasculature where these C-peptide deposits evoke the low-grade inflammation so typical of what is commonly called insulin-resistance. GXXPG and XGXPG motif binding to the EBP induces interleukin-I beta mediated proliferation of vascular and connective tissue cells.

Pericytes, smooth muscle cells, fibroblasts, adipose tissue cells, pancreatic stellate cells, and others, together with endothelial cells, and circulating leucocytes, respond to binding of EBP to GxxPG or xGxPG bearing proteins and peptides by interleukin-1-beta mediated proliferation and low-grade inflammatory activation. Analysis of the human proteome shows that proteins with multiple GxxPG or xGxPG motifs are highly related to the extracellular matrix (ECM). Matrix proteins with multiple GxxPG or xGxPG sites include fibrillin-1, -2, and -3, elastin, fibronectin, laminin, and several tenascins and collagens.

Recent studies have shown that the Neu-1 component of the EBP complex is responsible for triggering cellular activation. EBP is present on many cell types, including various types of leukocytes, mesenchymal cells, vascular smooth muscle cells, and skin fibroblasts. Whereas the hexapeptide SEQ ID NO:41 (VGVAPG), a commonly repeated sequence in human elastin, is the most well-recognized ligand for this receptor, C-peptide, galectin-3, the amino acid sequence SEQ ID NO:25 (FRAAPLQGMLPGLLAPLRT) in human collagen 6 A3 (COL6A3, UIniprot identifier P1211) and the beta-2 loop of human choriogonadotropin (hCG) are now herein also recognized as also capable of binding to the EBP. In addition to SEQ ID NO:41 (VGVAPG), (all elastin-derived) peptides that follow the motif GXXPG or XGXPG (where X is a hydrophobic amino acid) display chemotaxis for monocytes in vitro (Bisaccia F, et al., Int. J. Pept. Protein Res. 1994; 44:332-341, Castiglione Morelli M A, et al., J. Pept. Res. 1997; 49:492-499). This is noteworthy, albeit not having been observed before, because primate C-peptide sequences do not contain the SEQ ID NO:41 (VGVAPG) sequence; however, primate C-peptide contain significant quantities of both GXXP, GXXPG and XGXPG motifs that show similar activities. C-peptide's GxxP, GxxPG and xGxPG interactions explain IL-1-beta involvement. C-peptide's GxxP, GxxPG and xGxPG interactions have until now been overlooked by those skilled in the art of diabetes or metabolic disorder research as well as by those skilled in the art of elastin peptide and extracellular matrix (ECM) research. This earlier unobserved fact explains the macrophage-predominant, IL-1-beta mediated chronic inflammatory disease process as seen in, for example, adipose tissue in patients suffering from diabetes type-2, it explains the intima thickening and smooth muscle cell proliferation seen in vessels of patients suffering from atherosclerosis, the direct insulitis and peri-islet inflammation around beta cells in the pancreas as seen in the early phases of diabetes, and many other disease manifestations of metabolic syndrome wherein the patients suffer from C-peptide overproduction and C-peptide deposits, likely as a consequence of over-eating. C-peptide's GxxP, GxxPG and xGxPG interactions also explain leucocyte involvement. In addition, IL-1-beta signaling results in the production of pro-inflammatory mediators that act in a feed-forward autocrine/paracrine manner in beta-cells and local innate immune cells to amplify these effects, amplified by the fact that circulating leucocytes show strong chemotaxis to GxxPG or xGxPG bearing proteins and peptides; again C-peptide will thus attract those cells to wherever C-peptide is present, and in situations of C-peptide overload or even C-peptide deposits, this will exacerbate disease. As indicated herein, the concept that C-peptide and degradation products thereof can drive a macrophage-predominant, chronic inflammatory disease process via its GxxPG and xGxPG motif is now elucidating the etiology of diabetes of all types and is applicable to all diseases that occur in vasculature-rich organs and tissues, including coronary artery disease, peripheral vascular disease, and aortic aneurysm.

By “peptide” the inventor includes not only molecules in which amino acid residues are joined by peptide (—CO—NH—) linkages, but also functionally equivalent molecules in which the peptide bond is reversed. Retro-inverse peptides are composed of D-amino acids assembled in a reverse order from that of the parent L-sequence, thus maintaining the overall topology of the native sequence. Such retro-inverso peptidomimetics may be made using methods known in the art, for example, such as those described in Meziere et al. (1997) J. Immunol. 159, 3230-3237, and Carver et al. (1997) Biopolymers. 1997 Apr. 15; 41(5):569-90, incorporated herein by reference. This approach involves making pseudopeptides containing changes involving the backbone, and not the orientation of side chains. Meziere et al. and Carver et al. (1997) show that these pseudopeptides are useful. Retro-inverse peptides are much more resistant to proteolysis. Retro-inversion is a way of protecting peptide substances against proteolysis. It entails retro-inverting those peptide bonds most susceptible to enzymatic hydrolysis by inverting the direction of the peptide bonds. The “retro-inverso peptides” are structural isomers of the reference peptides and as such preserve their biological activity while being more resistant to enzymatic hydrolysis. A peptidomimetic is a small protein-like chain designed to mimic a peptide. They typically arise from modification of an existing peptide in order to alter the molecule's properties. For example, they may arise from modifications to change the molecule's stability or biological activity (only useful when the peptide's biological activity is chiral). Chemically synthesized peptides generally have free N- and C-termini. N-terminal acetylation and C-terminal amidation reduce the overall charge of a peptide; therefore, its overall solubility might decrease. However, the stability of the peptide could also be increased because the terminal acetylation/amidation generates a closer mimic of the native protein. These modifications might increase the biological activity of a peptide and are herein also provided.

### Peptide Synthesis

Synthetic PG-domain or GxxP-type peptides such as SEQ ID NO:41 (VGVAPG), SEQ ID NO:138 (GVAPGV), SEQ ID NO:139 (VAPGVG), SEQ ID NO:140 (APGVGV), SEQ ID NO:141 (PGVGVA), SEQ ID NO:142 (GVGVAP), SEQ ID NO:60 (PGAIPG), SEQ ID NO:137 (LGTIPG), SEQ ID NO:32 (LGGGPGAG), SEQ ID NO:8 (GGGPGAG), SEQ ID NO:49 (GGGPGA), SEQ ID NO:38 (GGGP), SEQ ID NO:40 (GGGPG), SEQ ID NO:46 (GAGPG), SEQ ID NO:50 (GGGPE), SEQ ID NO:51 (GAIPG), SEQ ID NO:52 (GGVPG), SEQ ID NO:53 (GVAPG), SEQ ID NO:54 (YTTGKLPYGYGPGG), SEQ ID NO:55 (YGARPGVGVGIP), SEQ ID NO:56 (PGFGAVPGA), SEQ ID NO:57 (GVYPG), SEQ ID NO:58 (GFGPG), SEQ ID NO:59 (GVLPG), SEQ ID NO:51 (GAIPG), SEQ ID NO:60 (PGAIPG), SEQ ID NO:61 (PGAVGP), SEQ ID NO:62 (VGAMPG), SEQ ID NO:63 (VGSLPG), SEQ ID NO:64 (VGMAPG), SEQ ID NO:65 (VPGVG), SEQ ID NO:66 (IPGVG), SEQ ID NO:63 (VGSLPG), SEQ ID NO:41 (VGVAPG), SEQ ID NO:67 (VGVPG), SEQ ID NO:68 (AGAIPG), SEQ ID NO:69 (VPGV), SEQ ID NO:70 (LGITPG), SEQ ID NO:71 (GDNP), SEQ ID NO:72 (GAIP), SEQ ID NO:73 (GKVP), SEQ ID NO:74 (GVQY), SEQ ID NO:75 (GVLP), SEQ ID NO:76 (GVGP), SEQ ID NO:77 (GFGP), SEQ ID NO:78 (GGIP), SEQ ID NO:79 (GVAP), SEQ ID NO:80 (GIGP), SEQ ID NO:39 (GAGP), SEQ ID NO:81 (GGIPP), SEQ ID NO:82 (GQFP), SEQ ID NO:83 (GLSP), SEQ ID NO:84 (GPQP), SEQ ID NO:85 (GGPQP), SEQ ID NO:86 (GPQPG), SEQ ID NO:87 (GGPQPG), SEQ ID NO:88 (GIPP), SEQ ID NO:81 (GGIPP), SEQ ID NO:89 (GIPPA), SEQ ID NO:90 (GGIPPA), or retro-inverso variants thereof are synthesized according to classical solid phase synthesis. V14 peptide, a peptide reproducing the sequence of S-Gal interacting with elastin peptides bearing the PG-domain, in particular, the motif GxxP, is obtained from Neosystem (Strasbourg, France). Alternatively, V14 peptide and variants thereof are synthesised as described herein. Purity of the peptides is confirmed by high performance liquid chromatography and by fast atom bombardment mass spectrometry.

Traditionally, peptides are defined as molecules that consist of between 2 and 50 amino acids, whereas proteins are made up of 50 or more amino acids. In addition, peptides tend to be less well defined in structure than proteins and can adopt complex conformations known as secondary, tertiary, and quaternary structures. Functional distinctions may also be made between peptides and proteins. Peptides, however, may be subdivided into peptides that have few amino acids (e.g., 2 to 30-50), and polypeptides that have many amino acids (>50). Proteins are formed from one or more polypeptides joined together. Hence, proteins essentially are very large peptides. In fact, most researchers, as well as this disclosure, use the term peptide to refer specifically to peptides, or otherwise relatively short amino acid chains, with the term polypeptide being used to describe proteins, or chains of >50 or much more amino acids.

Treatment of Cultured Cells with C-Peptide or Fragments Thereof.

Cells may be plated at a density of 450 per mm 2 in a 24-well microplate or 32 mm diameter Petri dish and cultured for 2-4 days or in cultures as described above. On day 2 in culture, cells are treated with various C-peptides (preferably selected from Table 1) or peptide fragments thereof for 1 or 2 days. In an experiment, cells were treated with a combination of C-peptide (1 micro-M) or polyclonal anti-67 kDa elastin receptor antibody (anti-S-Gal antibody) (10 ng per ml) for 2 d. At the end of the treatment, cells may be trypsinized (0.25%) and the cell number determined with a Coulter counter. For determination of thymidine incorporation, cells are labeled with 50 micro-Ci of [methyl-3H] thymidine (3.2 TBq per mmol; Amersham) for the final 18 h of the treatment. Incorporated thymidine is determined as trichloroacetic acid-precipitable counts with a liquid scintillation spectrometer (Beckman LS9800). Binding may be antagonized by adding V32-peptide or V32-peptide fragments or V14 peptide or V14-peptide fragments.

Detection of the 67 kDa elastin receptor. To select for or confirm the presence of the 67 kDa elastin receptor in cells, reverse transcription—polymerase chain reaction is performed using cellular RNA and synthetic oligoprimers corresponding to the beta-galactosidase cDNA sequences upstream and downstream spanning the region between exons 2 and 5. The reaction is run for 40 cycles with denaturation at 90° C. for 1 min, annealing at 50° C. for 2 min, and extension at 72° C. for 5 min in a DNA Thermal Cycler (Perkin-Elmer Cetus). The polymerase chain reaction products are preferably analyzed on 1% agarose gel.

In describing protein or peptide composition, structure and function herein, reference is made to amino acids. In the present specification, amino acid residues are expressed by using the following abbreviations. Also, unless explicitly otherwise indicated, the amino acid sequences of peptides and proteins are identified from N-terminal to C-terminal, left terminal to right terminal, the N-terminal being identified as a first residue. Ala: alanine residue; Asp: aspartate residue; Glu: glutamate residue; Phe: phenylalanine residue; Gly: glycine residue; His: histidine residue; Ile: isoleucine residue; Lys: lysine residue; Leu: leucine residue; Met: methionine residue; Asn: asparagine residue; Pro: proline residue; Gln: glutamine residue; Arg: arginine residue; Ser: serine residue; Thr: threonine residue; Val: valine residue; Trp: tryptophan residue; Tyr: tyrosine residue; Cys: cysteine residue. The amino acids may also be referred to by their conventional one-letter code abbreviations; A=Ala; T=Thr; V=Val; C=Cys; L=Leu; Y=Tyr; I=Ile; N=Asn; P=Pro; Q=Gln; F=Phe; D=Asp; W=Trp; E=Glu; M=Met; K=Lys; G=Gly; R=Arg; S=Ser; and H=His.

Overview 1. Elastin degradation and elastin peptides with a GxxP motif are associated with vascular disease.

### Elastin-Derived Peptides and Elastin Receptor Complex (ERC) Mediated Vascular Disease

Activation of ERC by proteolytically degraded elastin peptides is associated with vascular disease.

Matrix ageing and vascular impacts: focus on elastin fragmentation. Duca L, et al; Cardiovasc Res. 2016 Jun. 1; 110(3):298-308.

Hellenthal F A, Buurman W A, Wodzig W K, Schurink G W. Biomarkers of AAA progression. Part 1: extracellular matrix degeneration. Nat Rev Cardiol 2009; 6: [symbol] 464-474.

[symbol]

Monocyte chemotactic activity in human abdominal aortic aneurysms: role of elastin degradation-peptides and [symbol] the 67-kD cell surface elastin receptor. Hance K A, et al; J Vasc Surg 2002; 35:254-261.

Elastin degradation is associated with progressive aortic stiffening and all-cause mortality in predialysis chronic kidney disease. Smith E R, et al; Hypertension. 2012 May; 59(5):973-8.

### Prototype Synthetic Elastin Peptide SEQ ID NO:41 (VGVAPG)

Evidence that interaction of SEQ ID NO:41 (VGVAPG) with ERC may cause atherosclerosis and is involved in macrophage chemotaxis and angiogenesis.

Elastin-derived peptides potentiate atherosclerosis through the immune Neu1-PI3Kγ pathway. Gayral S, et al; Cardiovasc Res. 2014 Apr. 1; 102(1):118-27.

Induction of macrophage chemotaxis by aortic extracts from patients with Marfan syndrome is related to elastin binding protein. Guo G, et al; PLoS One. 2011; 6(5): e20138.

Elastin-derived peptides enhance angiogenesis by promoting endothelial cell migration and tubulogenesis through upregulation of MT1-MMP. Robinet A, et al; J Cell Sci. 2005 Jan. 15; 118 (Pt 2):343-56.

### Proteolytically Degraded Elastin Peptides SEQ ID NO:143 (VPGVGISPEA) and SEQ ID NO:144 (GVAPGIGPGG)

Evidence that SEQ ID NO:143 (VPGVGISPEA) and SEQ ID NO:144 (GVAPGIGPGG) localize in human atherosclerotic lesions and that serum levels of SEQ ID NO:144 (GVAPGIGPGG) associate with acute myocardial infarction. Note: None of the below authors recognize the GxxP motif in SEQ ID NO:143 (VPGVGISPEA) and SEQ ID NO:144 (GVAPGIGPGG).

Acute Myocardial Infarction and Pulmonary Diseases Result in Two Different Degradation Profiles of Elastin as Quantified by Two Novel ELISAs. Skjøt-Arkil H, et al; PLoS One. 2013 Jun. 21; 8(6):e60936.

Additional elastin derived peptides that interact with ERC and have biological activity are extensively discussed in:
Degradation of tropoelastin by matrix metalloproteinases—cleavage site specificities and release of matrikines. Heinz A, et al; FEBS J. 2010 April; 277(8):1939-56.
   Overview 2. Non-Elastin Peptides that have a GxxP-Motif and are Associated with Vascular Disease. C-Peptide with Midportion SEQ ID NO:8 (GGGPGAG)

Evidence that C-peptide localizes in human atherosclerotic lesions, induces macrophage chemotaxis and angiogenesis, that C-peptide may cause atherosclerosis and that serum levels of C-peptide associate with overall, cardiovascular and diabetes mortality. Typical degradation products of C-peptide are SEQ ID NO:145 (VELGGGPGAGSLQP), SEQ ID NO:146 (LGGGPGAGSLQP) and SEQ ID NO:147 (LGGGPGAGS). Note: None of the below authors recognize the GxxP motif in C-peptide.

C-peptide co-localizes with macrophages in early arteriosclerotic lesions of diabetic subjects and induces monocyte chemotaxis in vitro. Marx N, et al; Arterioscler Thromb Vasc Biol. 2004 March; 24(3):540-5.

Proinsulin C-peptide prevents impaired wound healing by activating angiogenesis in diabetes. Lim Y C, et al; J Invest Dermatol. 2015 January; 135(1):269-78.

C-peptide promotes lesion development in a mouse model of arteriosclerosis. Vasic D, et al; J Cell Mol Med. 2012 April; 16(4):927-35.

Fasting serum C-peptide levels predict cardiovascular and overall death in nondiabetic adults. Patel N, et al; J Am Heart Assoc. 2012 December; 1(6): e003152.

C-peptide levels are associated with mortality and cardiovascular mortality in patients undergoing angiography: the LURIC study. Marx N, et al; Diabetes Care. 2013 March; 36(3):708-14.

Serum C-peptide levels and risk of death among adults without diabetes mellitus. Min J Y, Min K B.

CMAJ. 2013 Jun. 11; 185(9):E402-8.

Serum C-peptide levels as an independent predictor of diabetes mellitus mortality in non-diabetic individuals. Min J Y, Min K B. Eur J Epidemiol. 2013 September; 28(9):771-4.

Galectin-3 with N-terminal “collagen-like-stretch” SEQ ID NO:148 (AGAGGYPGASYPGAYPGQAPPGAYPGQAPPGAYPGAPGAYPGAPAPGVYPGPPSG)

Evidence that galectin-3 plasma levels associate with heart failure. Note: None of the below authors recognize the GxxP motif in galectin-3.

Galectin-3, a novel marker of macrophage activity, predicts outcome in patients with stable chronic heart failure. Van der Lok, D, et al; J Am Coll Cardiol 2007 49Supp1. A 98A[Abstract]

Predictive value of plasma galectin-3 levels in heart failure with reduced and preserved ejection fraction. de Boer R A, et al; Ann Med. 2011 February; 43(1):60-8.

Fibrillin-1 with Motif SEQ ID NO:149 (EGFEPG)

Evidence that interaction of SEQ ID NO:149 (EGFEPG) with ERC is involved in macrophage chemotaxis.

Induction of macrophage chemotaxis by aortic extracts of the mgR Marfan mouse model and a GxxPG-containing fibrillin-1 fragment. Guo G, et al; Circulation 2006; 114:1855-1862.

[symbol]

Laminin with Motif SEQ ID NO:137 (LGTIPG)

Evidence that laminin interacts via motif SEQ ID NO:137 (LGTIPG) with ERC and induces fibroblast and tumor cell chemotaxis.

The elastin receptor shows structural and functional similarities to the 67-kDa tumor cell laminin receptor. Mecham R P et al; J Biol Chem. 1989 Oct. 5; 264(28):16652-7.

TABLE 1
C-peptide, interspecies comparisons and alignments

| | Uniprot | C-peptide amino |
|---|---|---|
| Species | identifier | acid sequence |
| Human | >sp\|P01308\|57-87 | SEQ ID NO: 1 |
| | | (EAEDLQVGQVELGGGPGAGSLQPLALEGSLQ) |
| human variant | rs121908279 | SEQ ID NO: 150 |
| | | (EAEDLQVGQVEMGGGPGAGSLQPLALEGSLQ) |
| human variant | rs121908274 | SEQ ID NO: 151 |
| | | (EAEDLQVGQVELGGGPGAGSLQPLALERSLQ) |
| chimpanzee | >sp\|P30410\|57-87 | SEQ ID NO: 1 |
| | | (EAEDLQVGQVELGGGPGAGSLQPLALEGSLQ) |
| Gorilla | >sp\|Q6YK33\|57-87 | SEQ ID NO: 1 |
| | | (EAEDLQVGQVELGGGPGAGSLQPLALEGSLQ) |
| orangutan | >sp\|Q8HXV2\|57-87 | SEQ ID NO: 1 |
| | | (EAEDLQVGQVELGGGPGAGSLQPLALEGSLQ) |
| Gibbon | G1RSS5 | SEQ ID NO: 152 |
| | | (EAEDPQVGQVELGGGPGAGSLQPLALEGSLQ) |
| macaque | >sp\|P30406\|57-87 | SEQ ID NO: 152 |
| | | (EAEDPQVGQVELGGGPGAGSLQPLALEGSLQ) |
| green monkey | >sp\|P30407\|57-87 | SEQ ID NO: 152 |
| | | (EAEDPQVGQVELGGGPGAGSLQPLALEGSLQ) |
| mouse insulin 2 | >sp\|P01326\|57-87 | SEQ ID NO: 153 |
| | | (EVEDPQVAQLELGGGPGAGDLQTLALEVAQQ) |
| mouse insulin 1 | >sp\|P01325\|57-85 | SEQ ID NO: 167 |
| | | (EVEDPQVEQLELGGSPGDLQTLALEVARQ) |
| rat insulin 2 | >sp\|P01323\|57-87 | SEQ ID NO: 154 |
| | | (EVEDPQVAQLELGGGPGAGDLQTLALEVARQ) |
| rat insulin 1 | >sp\|P01322\|57-87 | SEQ ID NO: 155 |
| | | (EVEDPQVPQLELGGGPEAGDLQTLALEVARQ) |
| Horse | F6QQU6 | SEQ ID NO: 156 |
| | | (EAEDPQVGQEELGGGPGLGGLQPLALAGPQQ) |
| Horse | >sp\|P01310\|33-63 | SEQ ID NO: 157 |
| | | (EAEDPQVGEVELGGGPGLGGLQPLALAGPQQ) |
| Horse | Most horses | SEQ ID NO: 158 |
| | | (EAEDPQVGQVELGGGPGLGGLQPLALAGPQQ) |
| chinchilla | >sp\|P01327\|33-63 | SEQ ID NO: 159 |
| | | (ELEDPQVGQADPGVVPEAGRLQPLALEMTLQ) |
| Guinea pig | >sp\|P01329\|57-87 | SEQ ID NO: 160 |
| | | (ELEDPQVEQTELGMGLGAGGLQPLALEMALQ) |
| Rabbit | >sp\|P01311\|57-87 | SEQ ID NO: 161 |
| | | (EVEELQVGQAELGGGPGAGGLQPSALELALQ) |
| Bovine | >sp\|P01317\|57-82 | SEQ ID NO: 164 |
| | | (EVEGPQVGALELAGGPGAGGLEGPPQ) |
| Bovine | Fleckvieh variant | SEQ ID NO: 165 |
| | | (EVEGPQVGALELAGGLGAGGLEGPPQ) |
| Sheep | >sp\|P01318\|57-82 | SEQ ID NO: 164 |
| | | (EVEGPQVGALELAGGPGAGGLEGPPQ) |
| Pig | >sp\|P01315\|57-85 | SEQ ID NO: 166 |
| | | (EAENPQAGAVELGGGLGGLQALALEGPPQ) |
| Dog | >sp\|P01321\|57-87 | SEQ ID NO: 162 |
| | | (EVEDLQVRDVELAGAPGEGGLQPLALEGALQ) |
| Cat | >sp\|P06306\|57-87 | SEQ ID NO: 163 |
| | | (EAEDLQGKDAELGEAPGAGGLQPSALEAPLQ) |

Table 2 The presence of the elastin receptor binding motif GxxP (underlined) in vascular matrix proteins elastin and fibrillin and in C-peptides. Peptides are shown with their respective identifiers and amino acids are numbered as shown in the database UIniprot.

TABLE 2

| | |
|---|---|
| A, elastic | |
| fiber proteins | |
| Elastin, P15502, | ₅₀₁ SEQ ID NO: 170 |
| H. sapiens | (GLVPGVGVAPGVGVAPGVGVAPGVGLAPGVGVAPGVGVAPG) ₅₄₁ |
| Fibrillin-1, P35555, | ₄₁₁ SEQ ID NO: 168 (PVLPVPPGFPPGPQIPVPRP) ₄₃₀- |
| H. sapiens | ₂₁₉₁ SEQ ID NO: 169 (TCEEGFEPGPM) ₂₂₀₁ |
| Fibrillin-2, P35556, | ₄₂₁ SEQ ID NO: 171 (LPMGGIPGSAGSRPGGTGGN) ₄₄₀- |
| H. sapiens | ₂₂₃₇ SEQ ID NO: 172 (NCNEGFEPGPM) ₂₂₄₇ |
| B, C-peptides | |
| P01308, H. sapiens | ₅₇ SEQ ID NO: 1 (EAEDLQVGQVELGGGPGAGSLQPLALEGSLQ) ₈₇ |
| P30410, P. troglodytes | ₅₇ SEQ ID NO: 1 (EAEDLQVGQVELGGGPGAGSLQPLALEGSLQ) ₈₇ |
| Q6YK33, G. gorilla | ₅₇ SEQ ID NO: 1 (EAEDLQVGQVELGGGPGAGSLQPLALEGSLQ) ₈₇ |
| Q8HXV2, P. pygmaeus | ₅₇ SEQ ID NO: 1 (EAEDLQVGQVELGGGPGAGSLQPLALEGSLQ) ₈₇ |
| P01325, M. | |
| musculus;(Ins-1) | ₅₇ SEQ ID NO: 167 (EVEDPQVEQLELGGSPGDLQTLALEVARQ) ₈₅ |
| P01326, M. | |
| musculus;(Ins-2) | ₅₇ SEQ ID NO: 153 (EVEDPQVAQLELGGGPGAGDLQTLALEVAQQ) ₈₇ |
| P01322, R. | |
| norvegicus;(Ins-1) | ₅₇ SEQ ID NO: 155 (EVEDPQVPQLELGGGPEAGDLQTLALEVARQ) ₈₇ |
| P01323, R. | |
| norvegicus;(Ins-2) | ₅₇ SEQ ID NO: 154 (EVEDPQVAQLELGGGPGAGDLQTLALEVARQ) ₈₇ |
| Q62587, P. obesus | ₅₇ SEQ ID NO: 173 (GVDDPQMPQLELGGSPGAGDLRALALEVARQ) ₈₇ |
| G5C2F2, H. glaber | ₅₇ SEQ ID NO: 174 (ELENLQVGQAEPGMGLEAGGLQPLAQELALQ) ₈₇ |
| P01315, S. scrofa | ₅₇ SEQ ID NO: 166 (EAENPQAGAVELGGGLGGLQALALEGPPQ) ₈₅ |

### Further Identification of ERC-Docking Sites

The elastin-receptor-complex (ERC) is thought to cause human vascular disease by binding excess peptide ligands derived from proteolysis of extra-cellular-matrix (ECM) after aging or smoking. Novel ERC-ligands were identified, notably in well-known biomarkers of vascular disease C-peptide (induced with insulin by high blood-glucose) and NTproBNP (induced in cardiomyocyte stress). It is proposed that A) to investigate accumulation of ERC-ligands as central etiology of human vascular disease, B) to early detect vascular disease risk by testing for ERC-ligands arising from accumulated risks diet, lifestyle and aging, that may all result in human vascular disease.

Background: ERC is a complex of elastin binding protein (EBP), protective protein/cathepsin A and neuraminidase-1, found on leucocytes, fibroblasts and smooth muscle cells. ERC-ligands confirm to binding motifs xGxxPG or xxGxPG (G being glycine, P proline, x any amino acid), or xGxxPx if adapted to a type VIII beta-turn. Prototype ERC-ligand SEQ ID NO:41 (VGVAPG) and others, such as SEQ ID NO:197 (YGYGPG), SEQ ID NO:198 (YGARPG), SEQ ID NO:199 (FGAVPG), are derived by proteolysis from repeat areas in elastin. Others are SEQ ID NO:149 (EGFEPG) (fibrillin) and SEQ ID NO:137 (LGTIPG) (laminin). EBP separately binds galactosides. ERC-ligand binding to EBP is antagonized by V14 peptide. Circulating levels of ERC-ligands, generated from elastin proteolysis in aging or by smoking, have been associated with atherosclerosis, arterial stiffness, abdominal aortic aneurysms and myocardial infarction in humans, providing ample basis to explore early diagnosis, prevention and treatment of ERC-mediated vascular disease. A composite in silico model is available to dock ERC-ligands in EBP for structural analyses and candidate drug-development. In vitro, ERC-ligand/EBP structure-function relationship may be studied in human cells by testing leukocyte chemotaxis, and proliferation of smooth muscle cells. ERC-ligands induce atherosclerosis and resistance to insulin in mice allowing in vivo study of ERC-mediated vascular disease.

Identification of ERC-Ligand Motifs Derived by Proteolysis from Non-ECM Proteins.

A first find is C-peptide, a peptide derived by prohormone convertase cleavage (PC) from the pre-proinsulin gene and excreted in equimolar amounts with insulin. C-peptide carries the ERC-ligand motif SEQ ID NO:34 (LGGGPG). Ido et al how C-peptide fragments with core motif SEQ ID NO:8 (GGGPGAG) to mitigate glucose-induced vascular dysfunction in rats but do not recognize the ERC-ligand motif. C-peptide has been found atherogenic in mice and an independent marker of human vascular disease. Thus, finding a putative ERC-ligand SEQ ID NO:34 (LGGGPG) in C-peptide acutely links ERC-mediated vascular disease to high-circulating C-peptide levels. It surprisingly provides a common etiology of vascular disease after smoking as well as after diets high in glucose or starch, wherein both etiologies are causally linked to circulating ligands of ERC.

A second find is galectin-3 that has an N-terminal domain, susceptible to proteolysis, with putative ERC-ligand repeat motifs SEQ ID NO:44 (PGAYPG). Galectin-3 is an independent marker of human vascular disease as well as obesity that underlies vascular disease. As galectin-3 and EBP both bind galactosides and are causal to insulin resistance in mice, it is suggested that a second relationship of galectin-3 to EBP next to putative ERC-ligand-receptor interaction.

A third find is ERC-ligand peptide motif SEQ ID NO:45 (QGVLPA) in loop 2 of beta-chorionic gonadotropin (beta-hCG), expressed during pregnancy, which loop is nicked by proteolysis from beta-hCG and involved in immunomodulation and angiogenesis.

We docked newly found SEQ ID NO:34 (LGGGPG), SEQ ID NO:44 (PGAYPG) and SEQ ID NO:45 (QGVLPA), and prototype SEQ ID NO:41 (VGVAPG), in the in-silico model of EBP. All fit this composite model. Also, preliminary in-vitro results show inhibition of bioactivity of C-peptide by ERC-antagonists V14 peptide.

We then performed a further search for proteins with xGxxPG or xxGxPG motifs closely flanked by PC cleavage sites, to identify ERC-ligands in regulatory model elements rf fragments thereof that may derive from pro-proteins. SEQ ID NO:200 (GVGAPG), SEQ ID NO:186 (PLGSPG), SEQ ID NO:201 (DGAKPG), SEQ ID NO:202 (QGMLPG), and SEQ ID NO:196 (AGGAPG) were found in procalcitonin (PCT), amino-terminal pro-brain natriuretic peptide (NTproBNP), pro-opiomelanacortin (POMC), collagen 6A3 (COL6A3), and pyrin, respectively. PCT and NTproBNP each correlate with heart failure. POMC relates to regulation of feeding behavior and COL6A3 relates to adipocyte function in obesity and insulin resistance. Pyrin relates to innate immunity.

TABLE 3
Biomarkers of vascular disease that carry the elastin
receptor binding motif.

| Table 3 Biomarkers of vascular | | relevant | |
|---|---|---|---|
| disease that carry the elastin | | hexa- | in silico |
| receptor binding motif | name | peptide | fit in EBP |
| Multiple occurrences of | | | |
| docking motif | | | |
| SEQ ID NO: 216 | Elastin | SEQ ID | + |
| (VGVAPGVGVAPGVGVAPGVGL | | NO: 41 | |
| APGVGVAPGVGVAPGVGVAPG) | | (VGVAPG) | |
| SEQ ID NO: 203 (FGLVPGVGVA) | | SEQ ID | |
| | | NO: 214 | |
| | | (FGLVPG) | |
| SEQ ID NO: 144 (GVAPGIGPGG) | Elastin after | SEQ ID | |
| | MMP9/12 | NO: 215 | |
| | | (PGIGPG) | |
| SEQ ID NO: 205 | Galectin-3 | SEQ ID | + |
| (PPGAYPGQAPPGAYPGAPGAYP | | NO: 44 | |
| GAPAPG) | | (PGAYPG) | |
| Single occurrence of | | | |
| docking motif | | | |
| SEQ ID NO: 206 (TCEEGFEPGP) | Fibrillin-1 | SEQ ID | |
| | | NO: 149 | |
| | | (EGFEPG) | |
| SEQ ID NO: 207 (NPLGTIPGGN) | Laminin beta-1 | SEQ ID | |
| | | NO: 137 | |
| | | (LGTIPG) | |
| Single occurrence of | | | |
| docking motif regulatory | | | |
| model element peptide | | | |
| SEQ ID NO: 208 | proinsulin C- | SEQ ID | + |
| (RREAEDLQVGQVELGGGPGAGS | peptide | NO: 34 | |
| LQPLALEGSLQKR) | | (LGGGPG) | |
| SEQ ID NO: 209 | beta-hCG loop 2 | SEQ ID | |
| (RVLQGVLPALPQVVCNYR) | | NO: 45 | |
| | | (QGVLPA) | + |
| SEQ ID NO: 210 | Procalcitonin | SEQ ID | |
| (KRCGNLSTCMLGTYTQDFNKFH | | NO: 200 | |
| TFPQTAIGVGAPGKKR) | | (GVGAPG) | |
| SEQ ID NO: 211 | NT-proBNP | SEQ ID | |
| (RSHPLGSPGSASDLETSGLQEQR) | | NO: 186 | |
| | | (PLGSPG) | |
| SEQ ID NO: 212 | Pro- | SEQ ID | |
| (KREDVSAGEDCGPLPEGGPEPRS | opiomelanacortin | NO: 201 | |
| DGAKPGPREGKR) | | (DGAKPG) | |
| SEQ ID NO: 213 | Collagen 6A3 | SEQ ID | |
| (RAAPLQGMLPGLLAPLR) | | NO: 202 | |
| | | (QGMLPG) | |
| SEQ ID NO: 192 | Pyrin | SEQ ID | |
| (RRNASSAGRLQGLAGGAPGQKECR) | | NO: 196 | |
| | | (AGGAPG) | |

We found that three well-known circulating biomarkers of vascular disease, C-peptide, amino-terminal pro-B-type natriuretic peptide (NT-proBNP) and galectin-3, and others, share a little-known docking site with circulating elastin-derived-peptides (EDP). Through this docking site, EDP activate the elastin-receptor-complex (ERC) that is expressed on cells throughout the human arterial system. ERC contributes to elastin degradation and arterial wall remodeling. Experimental activation of ERC by EDP induces insulin resistance and atherosclerosis in mice. Excess EDP/ERC docking causes chemotaxis of human leukocytes and proliferation of human smooth muscle cells (SMC) and is associated with loss of arterial elasticity, atherosclerosis, increased arterial stiffness, abdominal aortic aneurysms and myocardial infarction in humans.

What is claimed is:

1. A method of treating a subject for acute systemic inflammation, the method comprising:
administering to the subject a peptide consisting of 5-20 amino acids,
wherein the peptide comprises at least one amino acid Q, wherein Q represents glutamine,
wherein the peptide is able to agonize an elastin receptor so as to initiate physiological activity produced by binding of human C-peptide to human elastin receptor, and
wherein the peptide comprises at least one human elastin receptor binding motif GxxPG,
wherein G represents glycine,
wherein P represents proline, and
wherein x is any amino acid,
so as to treat the subject for acute systemic inflammation.

2. The method according to claim 1, wherein the peptide consists of 5-15 amino acids.

3. The method according to claim 1, wherein the peptide consists of 5-12 amino acids.

4. The method according to claim 1, wherein the peptide consists of 5-9 amino acids.

5. The method according to claim 1, wherein the peptide is a retro-inverso variant peptide composed of D-amino acids assembled in a reverse order from that of a parent L-sequence peptides listed under SEQ ID NOs: 3, 14, 15, 16, 21, 22, 23, 24, 25, 26, 27, 28, 29, 43, 93, 94, 175, and 176.

6. The method according to claim 1, wherein the human elastin receptor binding motif GxxPG is repeated at least twice in the peptide.

7. The method according to claim 6, wherein a repeat is separated by a linker comprising at least one amino acid.

8. The method according to claim 6, wherein the human elastin receptor binding motif GxxPG is repeated three times in the peptide.

9. The method according to claim 8, wherein a repeat is separated by a linker comprising at least one amino acid.

10. A method of treating a subject for acute systemic inflammation, the method comprising:
administering to the subject a peptide to reduce acute systemic inflammation in the subject, wherein the peptide consists of any one of SEQ ID NOs: 3, 14, 15, 16, 21, 22, 23, 24, 25, 26, 27, 28, 29, 43, 93, 94, 175, and 176.
